(19) United States
(12) Patent Application Publication
(10) Pub. No.: US 2010/0047841 A1
(43) Pub. Date: Feb. 25, 2010

(54) SYNTHESIS OF DESACETOXYTUBULYSIN H AND ANALOGS THEREOF

(75) Inventors: Peter WIPF, Pittsburgh, PA (US); Zhiyong Wang, Pittsburgh, PA (US)

Correspondence Address: FOLEY AND LARDNER LLP;SUITE 500, 3000 K STREET NW, WASHINGTON, DC, 20007, US

(73) Assignee: University of Pittsburgh-of the Commonwealth System of Higher Education

(21) Appl. No.: 12/037,457

(22) Filed: Feb. 26, 2008

Related U.S. Application Data
(60) Provisional application No. 60/891,785, filed on Feb. 27, 2007.
(60) Provisional application No. 60/929,624, filed on Jul. 5, 2007.

(51) Int. Cl. C12Q 1/02; C07K 5/08; C12N 5/06; C07D 417/02

(52) U.S. Cl. 435/29; 530/331; 435/375; 546/209

(57) ABSTRACT

Compounds of formula I, XVI and XXI possess potent cell growth inhibitory activity. These compounds are described have therapeutic utility, particularly in the treatment of cancer as well as conditions and disorders related to uncontrolled cell growth:

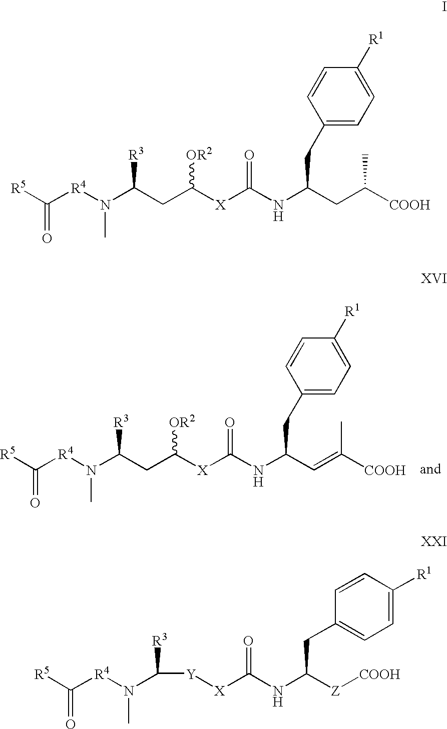

wherein the variables R¹, R², R³, R⁴, R⁵, X, Y and Z are described herein.

This application claims the benefit of U.S. Provisional Patent Applications No. 60/891,785, filed Feb. 27, 2007, and No. 60/929,624, filed Jul. 5, 2007, the respective contents of which are incorporated fully here by reference.

### STATEMENT REGARDING FEDERALLY SPONSORED RESEARCH

This invention was made with United States government support under grant number CA78039 awarded by the National Cancer Institute. The United States government may have certain rights in the invention.

### BACKGROUND OF THE INVENTION

The present invention relates generally to the fields of natural product chemistry and pharmaceutical chemistry. More specifically, the invention contemplates tubulysins and derivatives thereof, methods of synthesis thereof, and method for determination of antiproliferative activity thereof.

The information provided herein is intended solely to assist the understanding of the reader. None of the information provided nor references cited is admitted to be prior art to the present invention. Each of the references cited herein is incorporated in its entirety and for all purposes.

The tubulysins are naturally occurring agents that can be isolated, for example, from the myxobacterial strains Archangium gephyra and Angiococcus disciformis. which compounds have the general structure of Formula A (Table I) wherein substitution at R¹, R² and R³ (Table I) determines the type of tubulysin, e.g., tubulysin A, B, and the like (Sasse, F. et al., J. Antibiot. 2000, 53, 879; Steinmetz, H. et al., Angew. Chem., Int. Ed. 2004, 43, 4888). As Table I illustrates, the tubulysins are related structurally to known peptides, including the marine natural product dolastatins (Pettit, G. R., Pure Appl. Chem. 1994, 66, 2271; Kerbrat, P. et al., Eur. J. Cancer 2003, 39, 317) and the anticancer drug LU 103793 (de Arruda, M. et al., Cancer Res. 1995, 55, 3085; Marks, R. S. et al., Am. J. Clin. Oncol. 2003, 26, 336). In general terms, the “tubulysins” category is characterized by a tetrapeptide sequence that comprises the hydrophobic N-terminal amino acids, N-methylpipecolic acid (Mep) and isoleucine (Ile), which are adjacent to tubuvaline (Tuv) and the thiazole-containing residue, and which is terminated by tubuphenylalanine (Tup) or tubutyrosine (Tut).

TABLE I
Exemplary structures of tubulysins^{1,2}

| | |
|---|---|
| | Formula A |
| 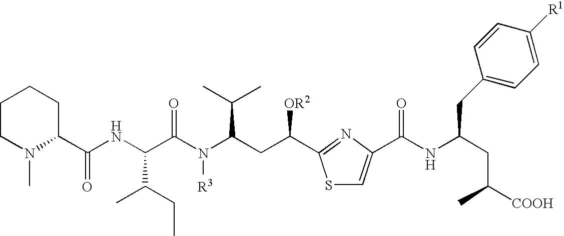 | |

| Tubulysin | R¹ | R² | R³ |
|---|---|---|---|
| A | OH | Acetyl | CH₂OC(O)CH₂CH(CH₃)₂ |
| B | OH | Acetyl | CH₂OC(O)CH₂CH₂CH₃ |
| C | OH | Acetyl | CH₂OC(O)CH₂CH₃ |
| D | H | Acetyl | CH₂OC(O)CH₂CH(CH₃)₂ |
| E | H | Acetyl | CH₂OC(O)CH₂CH₂CH₃ |
| F | H | Acetyl | CH₂OC(O)CH₂CH₃ |
| G | OH | Acetyl | CH₂OC(O)CH═C(CH₃)₂ |
| H | H | Acetyl | CH₂OC(O)CH₂CH(CH₃)₂ |
| I | OH | Acetyl | CH₂OC(O)CH₂CH(CH₃)₂ |
| U | H | Acetyl | H |
| V | H | H | H |
| Z | OH | H | H |

¹Tubulysins A-I as reported in Sasse et al. and in Steinmetz et al., supra.
²Tubulysins U, V and Z as reported in Dömling et al., Angew. Chem. Int. Ed. 2006, 45, 7235.

Tubulysins A-I (Table I) have IC₅₀ values of 0.3-7 ng/mL against the human cervix carcinoma, multidrug resistant cell line KB-V1 (Steinmetz et al., supra).

Without wishing to be bound by theory, it is currently thought that, similar to dolastatin, hemiasterlin, and HTI-286 (Huryn, D. M. & Wipf, P., In “Cancer Drug Design and Discovery” (Neidle, S., Ed.), Academic Press; in press), tubulysins inhibit the binding of vinblastine to tubulin, disrupt tubulin polymerization, and are likely to bind at the peptide site of the vinca domain of β-tubulin (Khalil, M. W. et al., ChemBioChem 2006, 7, 678; Kaur, G. et al, Biochem. J. 2006, 396, 235).

Dömling and coworkers synthesized stereoisomers of tubulysins U and V (Table I) using a three-component condensation approach (Dömling et al., Id.). Ellman and coworkers prepared tubulysin D by employing tert-butanesulfinamide chemistry (Peltier, H. M. et al., J. Am. Chem. Soc. 2006, 128, 16018). Zanda and coworkers reported a scalable synthesis of tubulysins U and V. (Sani, M. et al., Angew. Chem. Int. Ed. 2007, 46, 3526).

### SUMMARY OF THE INVENTION

Tubulysins possess potent cell growth inhibitory activity. They and their active analogs represent attractive leads for the development of pharmaceuticals for a variety of indications including, but not limited to, anticancer treatment. Accordingly, the present invention comprehends tubulysins and analogs thereof, methods for synthesis, methods for the assay of the antiproliferative effect of such tubulysins and analogs, and methods for screening for inhibitors of cellular proliferation directed at compounds of the invention.

In a preferred embodiment, the present invention relates to tubulysin analogs that have an N-alkyl group (e.g., methyl) functionality replacing the labile N,O-acetal functionality in, for example, tubulysin A-I (Table I). In this regard, the present invention provides compounds that are synthesized via the retrosynthetic approach, illustrated in Scheme 1:

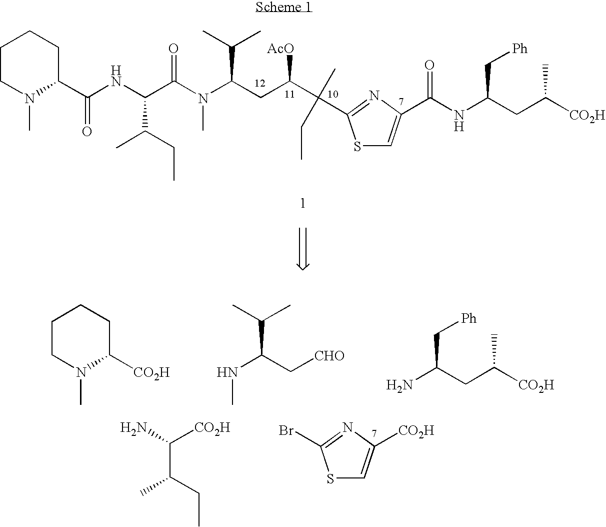

In one of its aspects, the invention provides a process for the preparation of a compound with structure of Formula I:

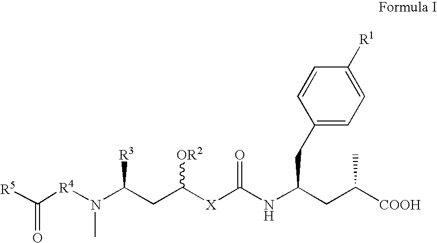

wherein R¹ is H or OH, R² is H or C(O)R⁶, R³ is C₁₋₆ alkyl, R⁴ is an amino acid selected from the group consisting of glycine, cysteine, alanine, histidine, asparagine, glutamine, arginine, threonine, valine, leucine, isoleucine, phenylalanine, tryptophan, serine, lysine, aspartic acid, methionine, glutamic acid, tyrosine, and optionally substituted derivatives thereof, R⁵ is selected from the group consisting of optionally substituted heteroalkyl, optionally substituted heterocycloalkyl, and optionally substituted heteroaryl, R⁶ is selected from the group consisting of H, optionally substituted alkyl, optionally substituted aryl, and optionally substituted heteroaryl, and X is optionally substituted arylene or optionally substituted heteroarylene. The process includes: (a) condensing a first protected amino acid with structure of Formula II:

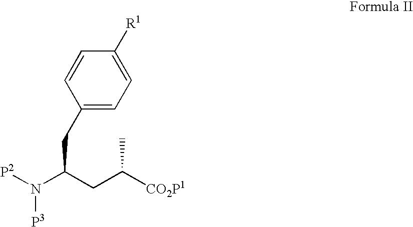

with a second protected amino acid with structure of Formula III:

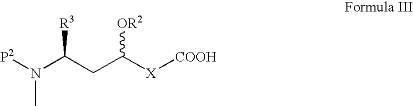

under conditions suitable to deprotect the first protected amino acid and to form a first compound with structure of Formula IV:

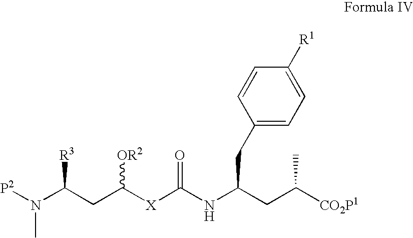

wherein P¹ is an acid protecting group, P² at each occurrence is independently an amine protecting group, and P³ is H or P²; (b) reacting the first compound with a third protected amino acid with structure P²—R⁴ under conditions suitable to deprotect the first compound and to form a second compound with structure of Formula V:

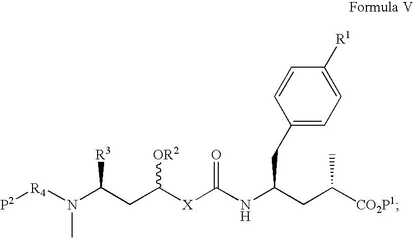

and (c) reacting the second compound with a reagent with structure of Formula VI (i.e., R⁵—COOH) or acid protected derivative thereof under conditions suitable to deprotect the second compound and form the compound having structure of Formula I.

In one aspect of the invention, a process is provided for preparing N-t-butyloxycarbonyl-N-methyl tubuvaline, which process includes: (a) reacting carbobenzoxyvaline under conditions suitable to form the methyl ester with structure of Formula IXa:

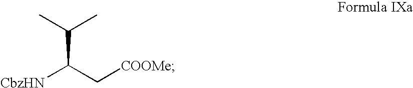

(b) reacting the methyl ester of step (a) under conditions suitable to form a t-butyldimethylsilyl ether with structure of Formula IXb:

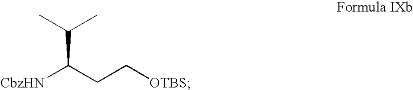

(c) reacting the t-butyldimethylsilyl ether under conditions suitable to form an alcohol with structure of Formula IXc:

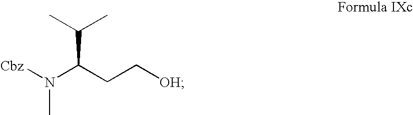

(d) reacting the alcohol under conditions suitable to form an aldehyde with structure of Formula IXd:

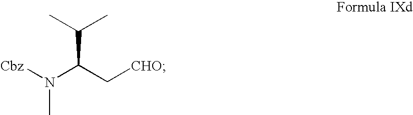

(e) reacting the aldehyde with a thiazole with structure of Formula IXe:

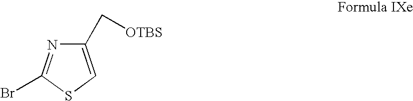

under Grignard conditions suitable to form epimeric compounds with structures of Formulae IXfₐ and IXf_{b}:

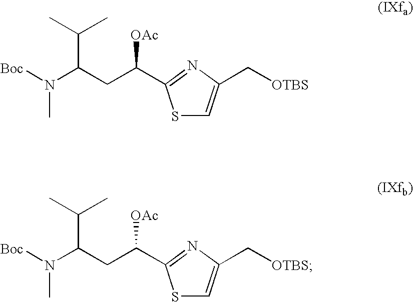

(f) reacting a compound resulting from step (e) under conditions suitable to form a deprotected alcohol with structure of either of Formulae IXgₐ and IXg_{b}:

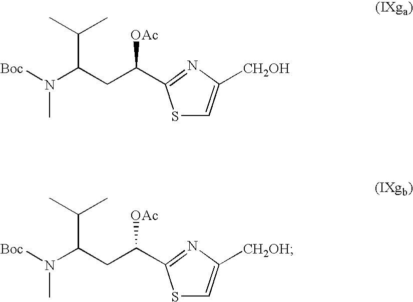

(g) reacting an alcohol resulting from step (f) under conditions suitable to form a N-t-butyloxycarbonyl-N-methyl tubuvaline derivative with structure of either of Formulae IXhₐ and IXh_{b}:

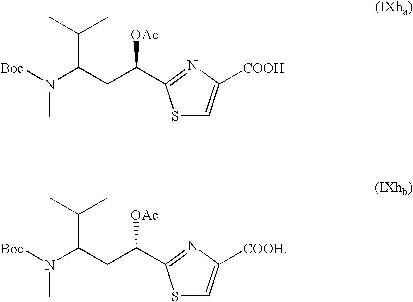

In yet another aspect, the invention comprehends the preparation of N¹⁴-desacetoxytubulysin H, having the structure of Formula X:

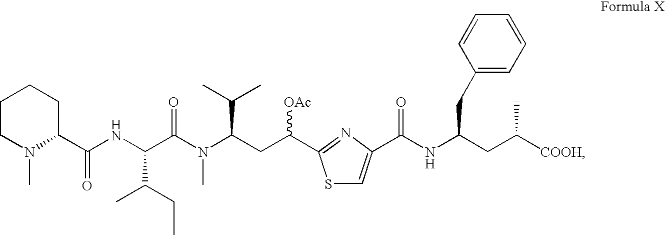

by a process that includes: (a) condensing a first protected amino acid with structure of Formula XI:

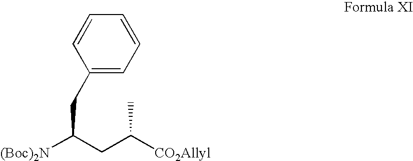

with a second protected amino acid with structure of Formula XII:

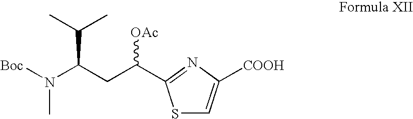

under conditions suitable to deprotect the first protected amino acid and to form a first compound with the structure of Formula XIII:

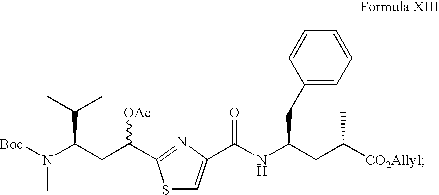

reacting the first compound with a protected isoleucyl reactant under conditions suitable to deprotect the first compound and to form a second compound with the structure of Formula XIV:

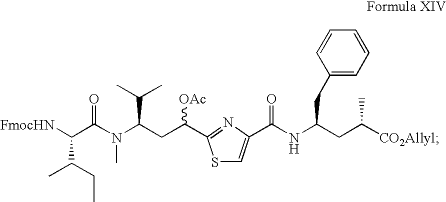

(c) reacting the second compound with acid protected 1-methylpiperidine-2-carboxylic acid under conditions suitable to deprotect the second compound and to form the N¹⁴-desacetoxytubulysin H with structure of Formula X.

In still another aspect, the invention provides the compounds of N¹⁴-desacetoxytubulysin H, or pharmaceutically acceptable salts thereof, having the structure of Formulae Xₐ or X_{b}:

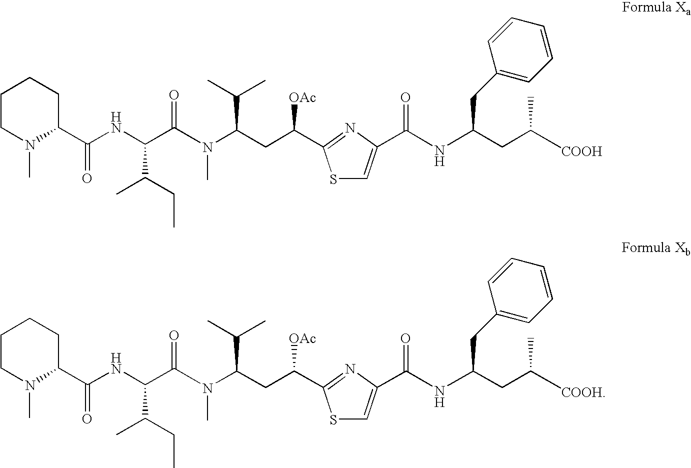

In yet another aspect, the invention provides a process for the preparation of a compound having the structure of Formula XV:

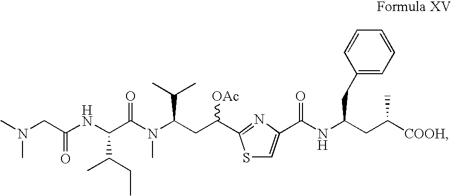

which process includes: (a) condensing a first protected amino acid with structure of Formula XI:

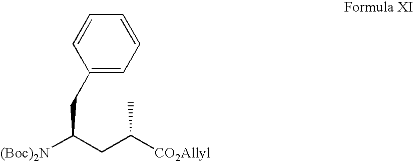

with a second protected amino acid with structure of Formula XII:

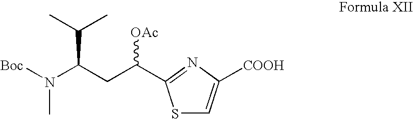

under conditions suitable to deprotect the first protected amino acid and to form a first compound with the structure of Formula XIII:

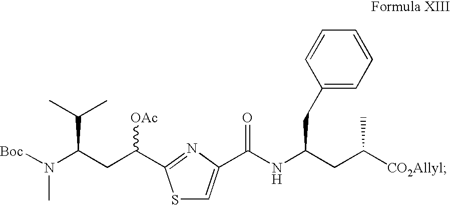

(b) reacting the first compound with a protected isoleucyl reactant under conditions suitable to deprotect the first compound and to form a second compound with the structure of Formula XIV:

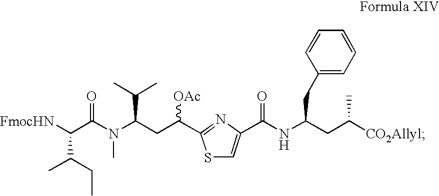

and (c) reacting the second compound with 2-(dimethylamino)acetic acid or acid protected derivative thereof under conditions suitable to deprotect the second compound and to form the compound with structure of Formula XV.

The present invention also relates to compounds, as well as to pharmaceutically acceptable salts thereof, that conform in structure to Formulae XVₐ or XV_{b}:

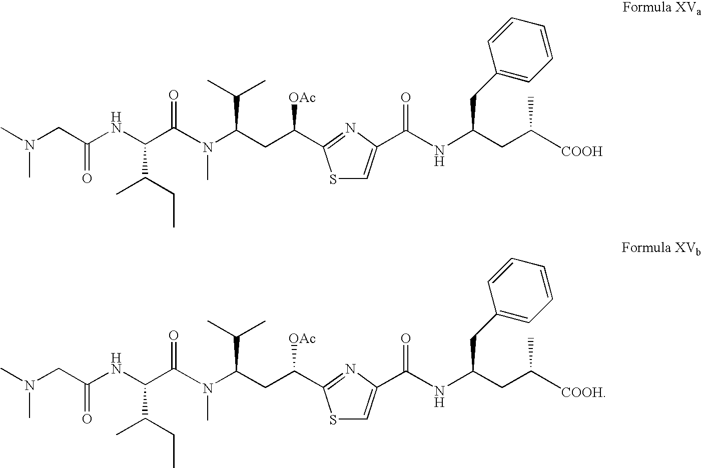

In still another aspect, the invention provides a method for inhibiting cellular proliferation, which includes contacting a cell with a compound having the structure of Formula I:

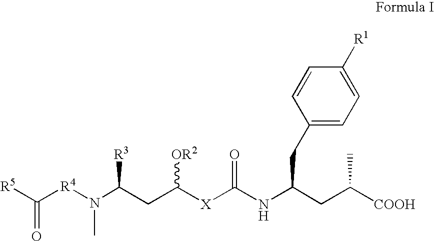

or pharmaceutically acceptable salt thereof, wherein R¹ is H or OH, R² is H or C(O)R⁶, R³ is C₁₋₆ alkyl, R⁴ is an amino acid selected from the group consisting of glycine, cysteine, alanine, histidine, asparagine, glutamine, arginine, threonine, valine, leucine, isoleucine, phenylalanine, tryptophan, serine, lysine, aspartic acid, methionine, glutamic acid, tyrosine, and optionally substituted derivatives thereof, R⁵ is selected from the group consisting of optionally substituted heteroalkyl, optionally substituted heterocycloalkyl, and optionally substituted heteroaryl, R⁶ is selected from the group consisting of H, optionally substituted alkyl, optionally substituted aryl, and optionally substituted heteroaryl; and X is optionally substituted arylene or optionally substituted heteroarylene.

In another aspect, the invention provides a method of screening for an inhibitor of cell proliferation. The inventive method includes: (a) determining, in the presence and in the absence of a test compound and a cell, respectively, a level of proliferation for the cell; (b) comparing the determined level in the presence and in the absence of the test compound; and then (c) ascertaining whether the test compound inhibits cell proliferation, where the test compound has the structure of Formula I:

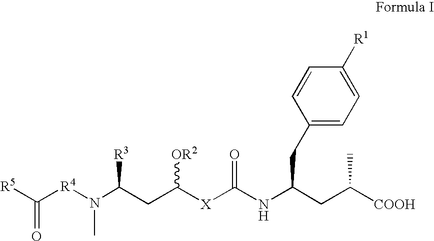

wherein: R¹ is H or OH, R² is H or C(O)R⁶, R³ is C₁₋₆ alkyl; R⁴ is an amino acid selected from the group consisting of glycine, cysteine, alanine, histidine, asparagine, glutamine, arginine, threonine, valine, leucine, isoleucine, phenylalanine, tryptophan, serine, lysine, aspartic acid, methionine, glutamic acid, tyrosine, and optionally substituted derivatives thereof, R⁵ is selected from the group consisting of optionally substituted heteroalkyl, optionally substituted heterocycloalkyl, and optionally substituted heteroaryl, R⁶ is selected from the group consisting of H, optionally substituted alkyl, optionally substituted aryl, and optionally substituted heteroaryl, and X is optionally substituted arylene or optionally substituted heteroarylene.

In another aspect, the invention provides a process for the preparation of a compound with structure of Formula XVI:

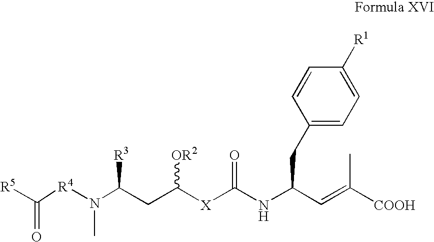

wherein R¹ is H or OH, R² is H or C(O)R⁶, R³ is C₁₋₆ alkyl, R⁴ is an amino acid selected from the group consisting of glycine, cysteine, alanine, histidine, asparagine, glutamine, arginine, threonine, valine, leucine, isoleucine, phenylalanine, tryptophan, serine, lysine, aspartic acid, methionine, glutamic acid, tyrosine, and optionally substituted derivatives thereof, R⁵ is selected from the group consisting of optionally substituted heteroalkyl, optionally substituted heterocycloalkyl, and optionally substituted heteroaryl, R⁶ is selected from the group consisting of H, optionally substituted alkyl, optionally substituted aryl, and optionally substituted heteroaryl, and X is optionally substituted arylene or optionally substituted heteroarylene. The process includes: (a) condensing a first protected amino acid with structure of Formula XVII:

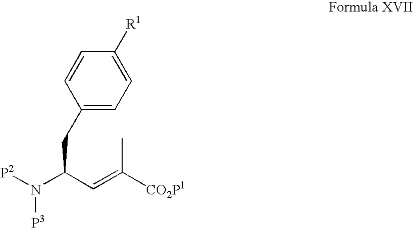

with a second protected amino acid with structure of Formula III:

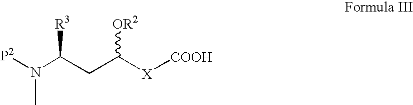

under conditions suitable to deprotect the first protected amino acid and to form a first compound with structure of Formula XVIII:

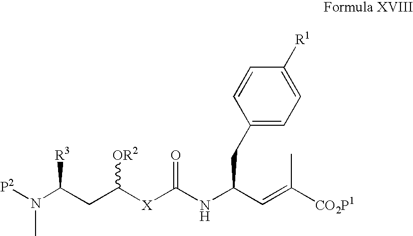

wherein P¹ is an acid protecting group, P² at each occurrence is independently an amine protecting group, and P³ is H or P²; (b) reacting the first compound with a third protected amino acid with structure P²—R⁴ under conditions suitable to deprotect the first compound and to form a second compound with structure of Formula XIX:

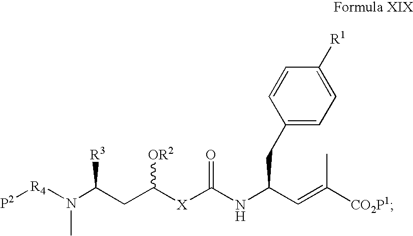

and (c) reacting the second compound with a reagent with structure of Formula VI (i.e., R⁵—COOH) or acid protected derivative thereof under conditions suitable to deprotect the second compound and form the compound having structure of Formula XVI. Unsaturated protected amino acids having the structure of Formula XVII are available by a variety of synthetic strategies known to one of skill of the art (e.g., Wipf, P., et al., Org. Let., 2004, 22:4057-4060).

In still another aspect, the invention provides compounds, or pharmaceutically acceptable salts thereof, with structure of Formula XVI:

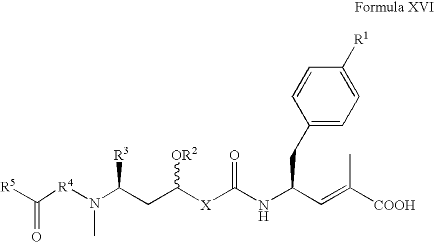

wherein R¹ is H or OH, R² is H or C(O)R⁶, R³ is C₁₋₆ alkyl, R⁴ is an amino acid selected from the group consisting of glycine, cysteine, alanine, histidine, asparagine, glutamine, arginine, threonine, valine, leucine, isoleucine, phenylalanine, tryptophan, serine, lysine, aspartic acid, methionine, glutamic acid, tyrosine, and optionally substituted derivatives thereof, R⁵ is selected from the group consisting of optionally substituted heteroalkyl, optionally substituted heterocycloalkyl, and optionally substituted heteroaryl, R⁶ is selected from the group consisting of H, optionally substituted alkyl, optionally substituted aryl, and optionally substituted heteroaryl, and X is optionally substituted arylene or optionally substituted heteroarylene.

In still another aspect, the invention provides compounds of N¹⁴-desacetoxytubulysin H, or pharmaceutically acceptable salts thereof, having the structure of Formulae XXₐ or XX_{b}:

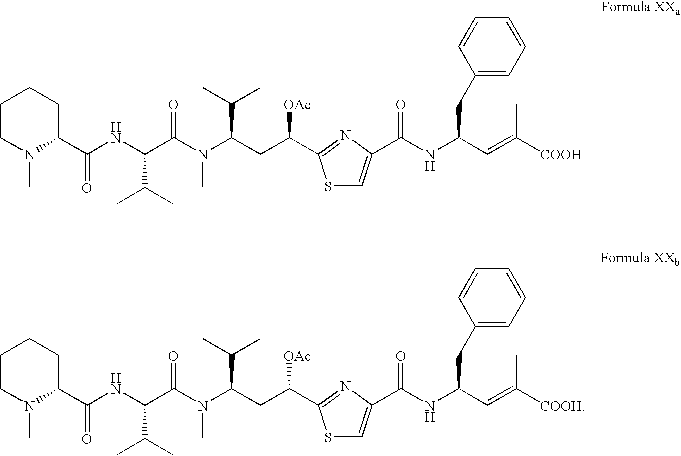

In another aspect, the invention provides a process for the preparation of a compound with structure of Formula XXI:

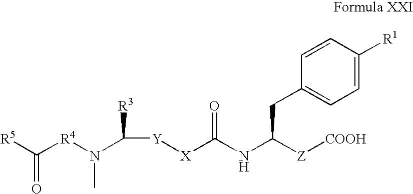

wherein R¹ is H or OH, Y is alkylene alkenylene or alkynylene, Z is optionally substituted alkylene or optionally substituted alkenylene, R³ is C₁₋₆ alkyl, R⁴ is an amino acid selected from the group consisting of glycine, cysteine, alanine, histidine, asparagine, glutamine, arginine, threonine, valine, leucine, isoleucine, phenylalanine, tryptophan, serine, lysine, aspartic acid, methionine, glutamic acid, tyrosine, and optionally substituted derivatives thereof, R⁵ is selected from the group consisting of optionally substituted heteroalkyl, optionally substituted heterocycloalkyl, and optionally substituted heteroaryl, and X is optionally substituted arylene or optionally substituted heteroarylene. The process includes: (a) condensing a first protected amino acid with structure of Formula II:

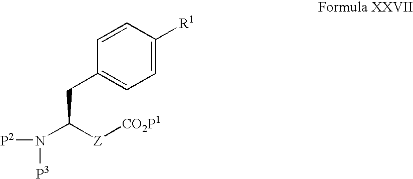

with a second protected amino acid with structure of Formula XXII:

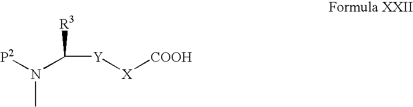

under conditions suitable to deprotect the first protected amino acid and to form a first compound with structure of Formula XXVIII:

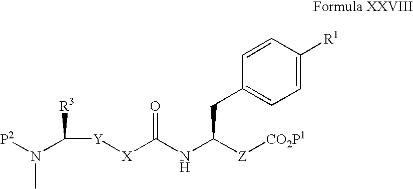

wherein P¹ is an acid protecting group, P² at each occurrence is independently an amine protecting group, and P³ is H or P²; (b) reacting the first compound with a third protected amino acid with structure P²—R⁴ under conditions suitable to deprotect the first compound and to form a second compound with structure of Formula XXIX:

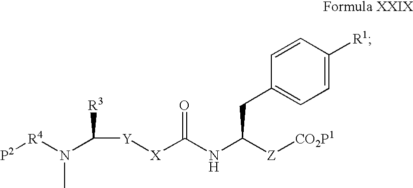

and (c) reacting the second compound with a reagent with structure of Formula VI (i.e., R⁵—COOH) or acid protected derivative thereof under conditions suitable to deprotect the second compound and form the compound having structure of Formula XXI. Protected amino acids having the structure of Formula XXII wherein Y is alkylene are available by a variety of synthetic strategies known to one of skill of the art including e.g., Barton deoxygenation of compounds having the structure of Formulae IXfₐ or IXf_{b} (Ishiwata, H., et al., Tetrahedron, 1994, 45:12853-12882), optionally with dehydrogenation to afford compounds wherein Y is alkenylene.

In still another aspect, the invention provides a process for the preparation of a compound with structure of Formula XXXV

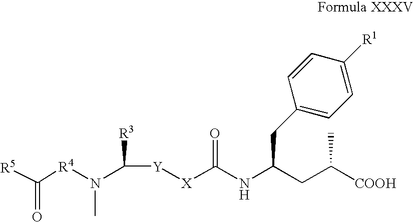

wherein R¹ is H or OH, Y is alkylene alkenylene or alkynylene, Z is optionally substituted alkylene or optionally substituted alkenylene, R³ is C₁₋₆ alkyl, R⁴ is an amino acid selected from the group consisting of glycine, cysteine, alanine, histidine, asparagine, glutamine, arginine, threonine, valine, leucine, isoleucine, phenylalanine, tryptophan, serine, lysine, aspartic acid, methionine, glutamic acid, tyrosine, and optionally substituted derivatives thereof, R⁵ is selected from the group consisting of optionally substituted heteroalkyl, optionally substituted heterocycloalkyl, and optionally substituted heteroaryl, and X is optionally substituted arylene or optionally substituted heteroarylene. The process includes: (a) condensing a first protected amino acid with structure of Formula II:

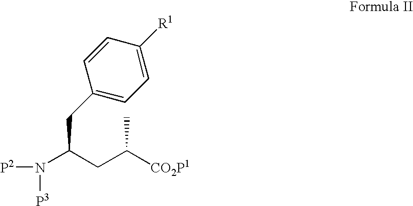

with a second protected amino acid with structure of Formula XXII:

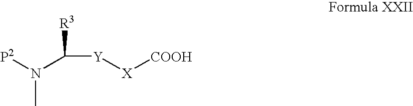

under conditions suitable to deprotect the first protected amino acid and to form a first compound with structure of Formula XIII:

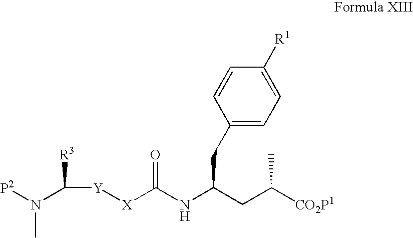

wherein P¹ is an acid protecting group, P² at each occurrence is independently an amine protecting group, and P³ is H or P²; (b) reacting the first compound with a third protected amino acid with structure P²—R⁴ under conditions suitable to deprotect the first compound and to form a second compound with structure of Formula XIV:

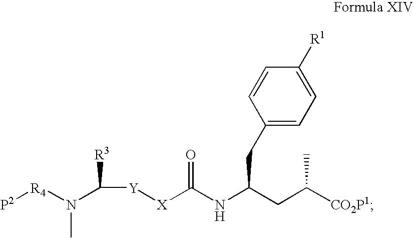

and (c) reacting the second compound with a reagent with structure of Formula VI (i.e., R⁵—COOH) or acid protected derivative thereof under conditions suitable to deprotect the second compound and form the compound having structure of Formula XXXV.

In still another aspect, the invention comprehends the preparation of N¹⁴-desacetoxytubulysin H, having the structure of Formula XXX:

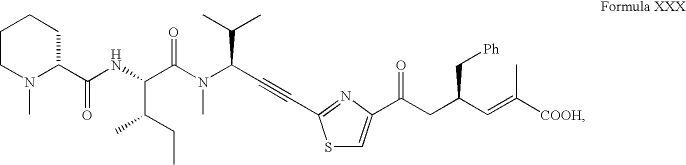

by a process that includes: (a) condensing a first protected amino acid with structure of Formula XVII:

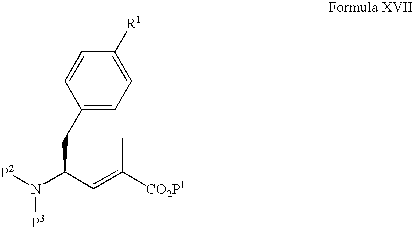

with a second protected amino acid with structure of Formula XXXVI:

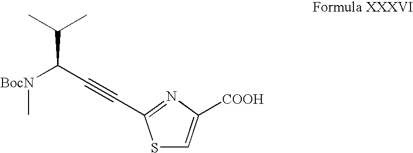

under conditions suitable to deprotect the first protected amino acid and to form a first compound with the structure of Formula XXXVII:

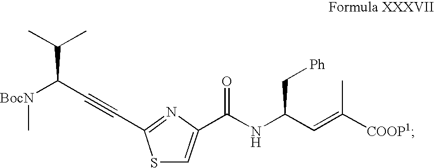

(b) reacting the first compound with a protected isoleucyl reactant under conditions suitable to deprotect the first compound and to form a second compound with the structure of Formula XXXVIII:

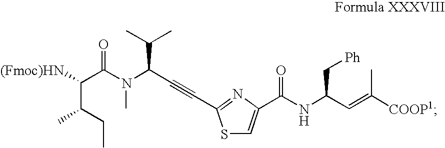

(c) reacting the second compound with acid protected 1-methylpiperidine-2-carboxylic acid under conditions suitable to deprotect the second compound, followed by removal of acid protecting group (P¹) to form the N¹⁴-desacetoxytubulysin H with structure of Formula XXX.

In yet another aspect, the invention comprehends the preparation of N¹⁴-desacetoxytubulysin H, having the structure of Formula XXX:

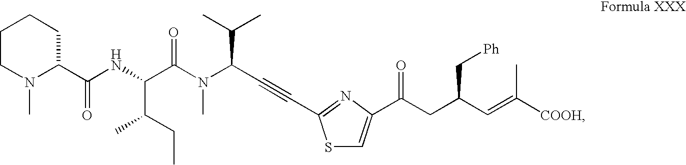

by a process that includes: (a) reacting the acetylide with structure of Formula XXXIX:

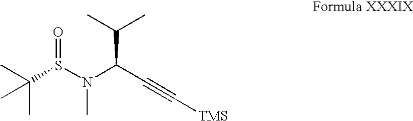

with a protected isoleucyl reactant under conditions suitable to deprotect the acetylide, followed by removal of the trimethylsilyl protecting group to form a first compound with the structure of Formula XXXX:

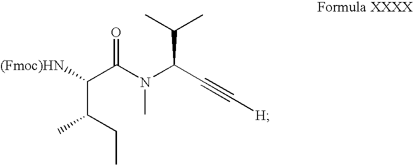

(b) reacting the first compound with the pentafluorophenyl ester of 1-methylpiperidine-2-carboxylic acid under conditions suitable to deprotect the first compound and to form a second compound with the structure of Formula XXXXI:

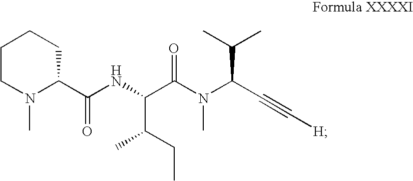

(c) coupling the second compound with the structure of Formula XXXXII

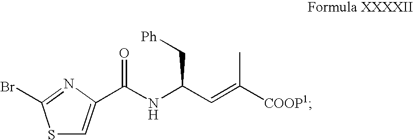

under suitable conditions, followed by removal of acid protecting group (P¹) to form the N¹⁴-desacetoxytubulysin H with structure of Formula XXX.

In yet another aspect, the invention comprehends the preparation of N¹⁴-desacetoxytubulysin H, having the structure of Formula XXV:

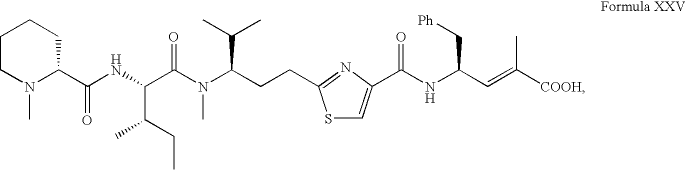

by a process that includes: (a) condensing a first protected amino acid with structure of Formula XXXXIII:

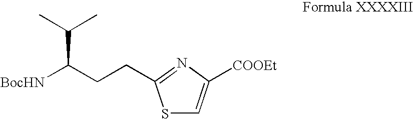

with a second protected amino acid with structure of Formula XXXXIV:

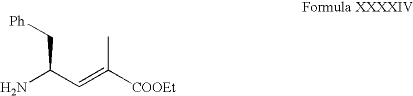

under conditions suitable to deprotect the first protected amino acid and to form a first compound with the structure of Formula XXXXV:

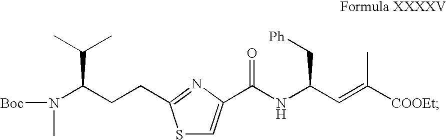

(b) reacting the first compound with a protected isoleucyl reactant under conditions suitable to deprotect the first compound and to form a second compound with the structure of Formula XXXXVI:

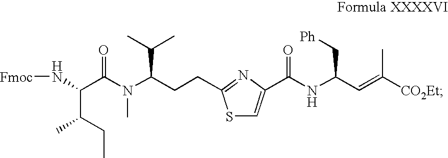

(c) reacting the second compound with the pentafluorophenyl ester of 1-methylpiperidine-2-carboxylic acid under conditions suitable to deprotect the second compound, followed by hydrolysis of ethyl ester group to give the compound with structure of Formula XXV.

In still another aspect, the invention provides compounds, or pharmaceutically acceptable salts thereof, with structure of Formula XXI:

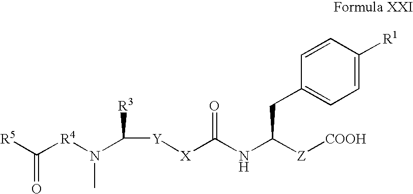

wherein R¹ is H or OH, Y is alkylene alkenylene or alkynylene, Z is optionally substituted alkylene or optionally substituted alkenylene, R³ is C₁₋₆ alkyl, R⁴ is an amino acid selected from the group consisting of glycine, cysteine, alanine, histidine, asparagine, glutamine, arginine, threonine, valine, leucine, isoleucine, phenylalanine, tryptophan, serine, lysine, aspartic acid, methionine, glutamic acid, tyrosine, and optionally substituted derivatives thereof, R⁵ is selected from the group consisting of optionally substituted heteroalkyl, optionally substituted heterocycloalkyl, and optionally substituted heteroaryl, and X is optionally substituted arylene or optionally substituted heteroarylene. In some embodiments, Y is ethylene. In some embodiments, Y is ethenylene or ethynylene.

In still another aspect, the invention provides the compounds of N¹⁴-desacetoxytubulysin H, or pharmaceutically acceptable salts thereof, having the structure of Formulae XXV, XXVI, XXX, XXXI, XXXII, XXXIII, or XXXIV:

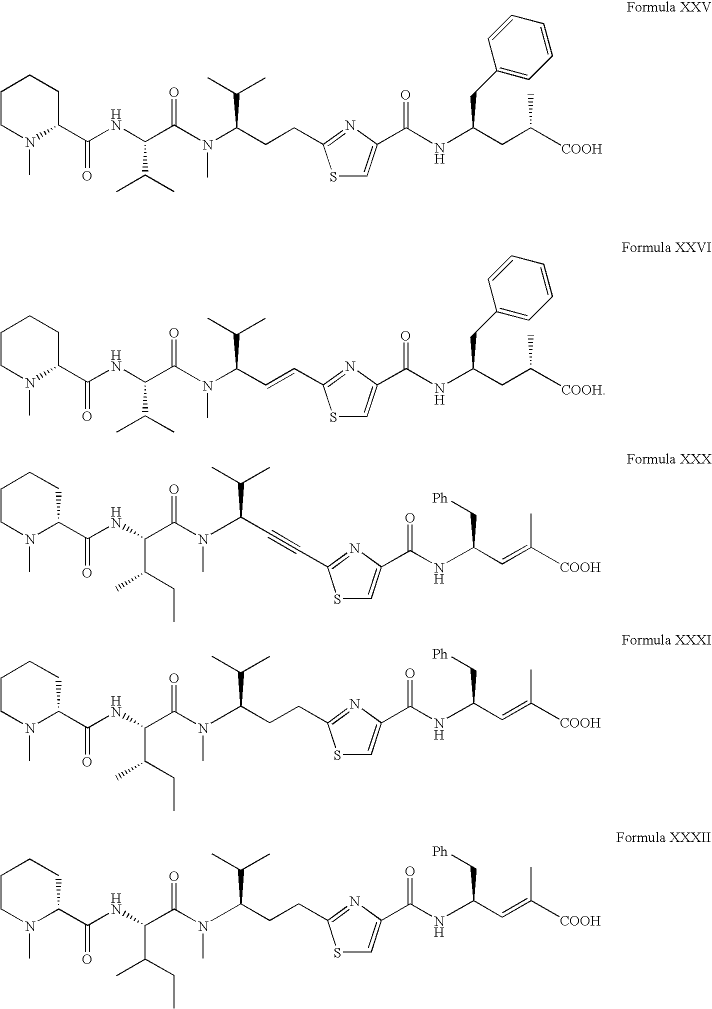

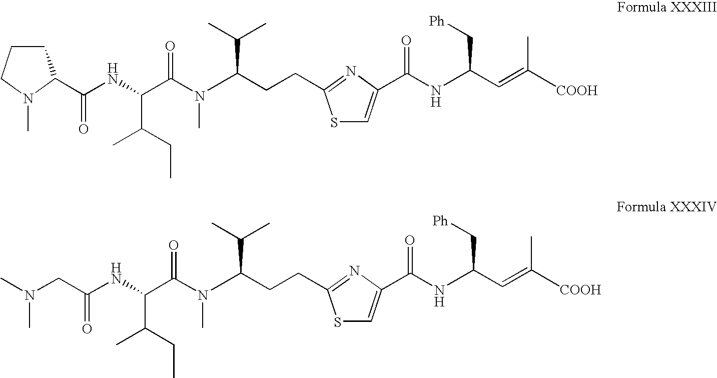

In still another aspect, the invention provides a method for inhibiting cellular proliferation, which method includes contacting a cell with a compound having the structure of Formula XVI

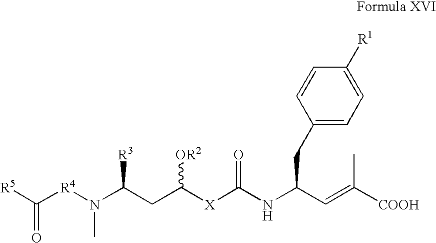

wherein R¹ is H or OH, R² is H or C(O)R⁶, R³ is C₁₋₆ alkyl, R⁴ is an amino acid selected from the group consisting of glycine, cysteine, alanine, histidine, asparagine, glutamine, arginine, threonine, valine, leucine, isoleucine, phenylalanine, tryptophan, serine, lysine, aspartic acid, methionine, glutamic acid, tyrosine, and optionally substituted derivatives thereof, R⁵ is selected from the group consisting of optionally substituted heteroalkyl, optionally substituted heterocycloalkyl, and optionally substituted heteroaryl, R⁶ is selected from the group consisting of H, optionally substituted alkyl, optionally substituted aryl, and optionally substituted heteroaryl; and X is optionally substituted arylene or optionally substituted heteroarylene.

In still another aspect, the invention provides a method for inhibiting cellular proliferation, which method includes contacting a cell with a compound having the structure of Formula XXI

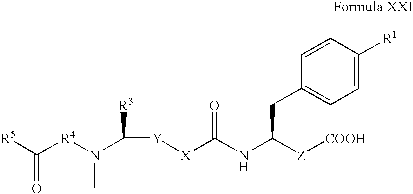

wherein R¹ is H or OH, Y is alkylene alkenylene or alkynylene, Z is optionally substituted alkylene or optionally substituted alkenylene, R³ is C₁₋₆ alkyl, R⁴ is an amino acid selected from the group consisting of glycine, cysteine, alanine, histidine, asparagine, glutamine, arginine, threonine, valine, leucine, isoleucine, phenylalanine, tryptophan, serine, lysine, aspartic acid, methionine, glutamic acid, tyrosine, and optionally substituted derivatives thereof, R⁵ is selected from the group consisting of optionally substituted heteroalkyl, optionally substituted heterocycloalkyl, and optionally substituted heteroaryl, and X is optionally substituted arylene or optionally substituted heteroarylene.

In another aspect, the invention provides a method of screening for an inhibitor of cell proliferation. The inventive method includes: (a) determining, in the presence and in the absence of a test compound and a cell, respectively, a level of proliferation for the cell; (b) comparing the determined level in the presence and in the absence of the test compound; and then (c) ascertaining whether the test compound inhibits cell proliferation, where the test compound has the structure of Formula XVI:

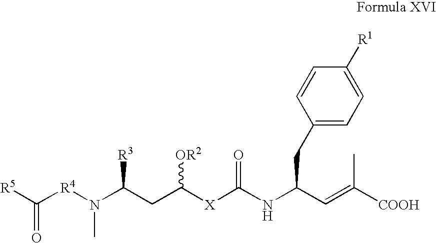

wherein R¹ is H or OH, R² is H or C(O)R⁶, R³ is C₁₋₆ alkyl, R⁴ is an amino acid selected from the group consisting of glycine, cysteine, alanine, histidine, asparagine, glutamine, arginine, threonine, valine, leucine, isoleucine, phenylalanine, tryptophan, serine, lysine, aspartic acid, methionine, glutamic acid, tyrosine, and optionally substituted derivatives thereof, R⁵ is selected from the group consisting of optionally substituted heteroalkyl, optionally substituted heterocycloalkyl, and optionally substituted heteroaryl, R⁶ is selected from the group consisting of H, optionally substituted alkyl, optionally substituted aryl, and optionally substituted heteroaryl, and X is optionally substituted arylene or optionally substituted heteroarylene.

In another aspect, the invention provides a method of screening for an inhibitor of cell proliferation. The inventive method includes: (a) determining, in the presence and in the absence of a test compound and a cell, respectively, a level of proliferation for the cell; (b) comparing the determined level in the presence and in the absence of the test compound; and then (c) ascertaining whether the test compound inhibits cell proliferation, where the test compound has the structure of Formula XXI

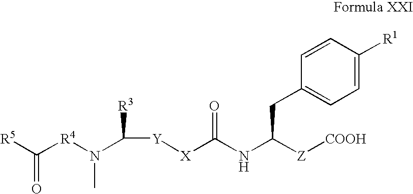

wherein R¹ is H or OH, Y is alkylene alkenylene or alkynylene, Z is optionally substituted alkylene or optionally substituted alkenylene, R³ is C₁₋₆ alkyl, R⁴ is an amino acid selected from the group consisting of glycine, cysteine, alanine, histidine, asparagine, glutamine, arginine, threonine, valine, leucine, isoleucine, phenylalanine, tryptophan, serine, lysine, aspartic acid, methionine, glutamic acid, tyrosine, and optionally substituted derivatives thereof, R⁵ is selected from the group consisting of optionally substituted heteroalkyl, optionally substituted heterocycloalkyl, and optionally substituted heteroaryl, and X is optionally substituted arylene or optionally substituted heteroarylene.

### DESCRIPTION OF THE FIGURES

FIG. 1. High-content analysis of mitotic arrest profiles in cells treated with microtubule-perturbing agents. Cells were treated in 384 well plates with ten two-fold dilutions of paclitaxel (PTX) (▪), vincristine (VCR) (□), N¹⁴-desacetoxytubulysin H (WZY-III-63C) (Δ), WZY-III-69A (♦), or WZY-III-64A (◯), and analyzed by high-content analysis for A) cell density, B) microtubule density, C) chromatin condensation and D) mitotic index. All agents enhanced mitotic index, nuclear condensation, and caused cell loss. The microtubule-destabilizing agent vincristine and WZY-III-63C showed initial increases in microtubule density that reversed at higher concentrations. In contrast, microtubule density increased steadily with the microtubule-stabilizing agent paclitaxel. Changes in all parameters were well correlated. N¹⁴-desacetoxytubulysin H (WZY-III-63C) was active in the picomolar range, compared with nanomolar activity for vincristine and paclitaxel. Data are the averages ±SEM of quadruplicate wells from a single experiment repeated three times with similar results.

FIG. 2. Concentration-dependent effects of N¹⁴-desacetoxytubulysin H (WZY-III-63C) on GTP-induced assembly of bovine brain tubulin as assessed by turbidimetry at 350 nm. A) The test agent was present at the beginning of the experiment. Numbers in parentheses next to the respective concentrations of WZY-III-63C denote the percent inhibition of assembly. B) The test agent was added after ˜7 min of GTP-induced assembly. The spikes in the curves are due to transient air bubbles. See Methods and Table IV for experimental details and results of IC₅₀ calculations.

FIG. 3. Concentration-dependent inhibition of binding of radiolabeled ligands to bovine brain tubulin. A) Inhibition of [³H]vinblastine binding. B) Inhibition of [³H]dolastatin binding. See Methods and Table IV for experimental details and results of IC₅₀ calculations.

FIG. 4. Summary of structure/activity-relationship (SAR) results.

### DETAILED DESCRIPTION OF THE INVENTION

In this description, a reference to a certain element, such as hydrogen (H), is meant to include all isotopes of that element. Thus, a group that is said to include hydrogen also includes deuterium and tritium, respectively.

Compounds of the present invention may have asymmetric centers and may occur, except when specifically noted, as mixtures of stereoisomers or as individual diastereomers, or enantiomers, with all isomeric forms being included in the present invention. Compounds of the present invention embrace all conformational isomers, including, for example, cis- or trans-conformations. Compounds of the present invention may also exist in one or more tautomeric forms, including both single tautomers and mixtures of tautomers.

The term “alkyl” refers to straight, branched chain, or cyclic hydrocarbyl groups including from 1 to about 20 carbon atoms. Alkyl includes straight chain alkyl groups such as methyl, ethyl, propyl, butyl, pentyl, hexyl, heptyl, octyl, nonyl, decyl, undecyl, dodecyl, and the like, and also includes branched chain isomers of straight chain alkyl groups, for example without limitation, —CH(CH₃)₂, —CH(CH₃)(CH₂CH₃), —CH(CH₂CH₃)₂, —C(CH₃)₃, —C(CH₂CH₃)₃, —CH₂CH(CH₃)₂, —CH₂CH(CH₃)(CH₂CH₃), —CH₂CH(CH₂CH₃)₂, —CH₂C(CH₃)₃, —CH₂C(CH₂CH₃)₃, —CH(CH₃)CH(CH₃)(CH₂CH₃), —CH₂CH₂CH(CH₃)₂, —CH₂CH₂CH(CH₃)(CH₂ CH₃), —CH₂CH₂CH(CH₂CH₃)₂, —CH₂CH₂C(CH₃)₃, —CH₂CH₂C(CH₂CH₃)₃, —CH(CH₃)CH₂CH(CH₃)₂, —CH(CH₃)CH(CH₃)CH(CH₃)₂, and the like. Thus, alkyl groups include primary alkyl groups, secondary alkyl groups, and tertiary alkyl groups. Preferred alkyl groups include alkyl groups having from 1 to 10 carbon atoms while even more preferred such groups have from 1 to 5 carbon atoms.

The phrase “substituted alkyl” refers to alkyl substituted at 1 or more, e.g., 1, 2, 3, 4, 5, or even 6 positions, with substitution as described herein.

The term “cycloalkyl” refers to an optionally substituted saturated or unsaturated non-aromatic monocyclic, bicyclic or tricyclic carbon ring systems of 3-10, more preferably 3-6, ring members per ring, such as cyclopropyl, cyclopentyl, cyclohexyl, adamantyl, and the like, having 1-3 optional substitutions as defined herein.

The term “alkylene” refers to divalent alkyl, and “substituted alkylene” refers to divalent substituted alkyl. Examples of alkylene include without limitation, ethylene (—CH₂—CH₂—).

The term “alkene” refers to straight, branched chain, or cyclic hydrocarbyl groups including from 2 to about 20 carbon atoms having at least one, preferably 1-3, more preferably 1-2, most preferably one, carbon to carbon double bond. “Substituted alkene” refers to alkene substituted at 1 or more, e.g., 1, 2, 3, 4, 5, or even 6 positions, with substitution as described herein.

The term “alkenylene” refers to divalent alkene. Examples of alkenylene include without limitation, ethenylene (—CH═CH—) and all stereoisomeric and conformational isomeric forms thereof. “Substituted alkenylene” refers to divalent substituted alkene.

The term “alkyne” refers to straight, or cyclic hydrocarbyl groups including from 2 to about 20 carbon atoms having at least one, preferably 1-3, more preferably 1-2, most preferably one, carbon to carbon triple bond. “Substituted alkyne” refers to alkyne substituted at 1 or more positions, with substitution as described herein.

The term “alkynylene” refers to divalent alkyne. Examples of alkynylene include without limitation, ethynylene (—C≡C—). “Substituted alkynylene” refers to divalent substituted alkyne.

Moieties of the present invention may be substituted with various atoms as described herein. As used here, “substitution” denotes an atom or group of atoms that has been replaced with another atom or group of atoms (i.e., substituent), and includes all levels of substitution, e.g. mono-, di-, tri-, tetra-, penta-, or even hex-substitution, where such substitution is chemically permissible. Substitutions can occur at any chemically accessible position and on any atom, such as substitution(s) on carbon and any heteroatom, preferably oxygen, nitrogen, or sulfur. For example, substituted moieties include those where one or more bonds to a hydrogen or carbon atom(s) contained therein are replaced by a bond to non-hydrogen and/or non-carbon atom(s). Substitutions can include, but are not limited to, a halogen atom such as F, Cl, Br, and I; an oxygen atom in groups such as hydroxyl groups, alkoxy groups, aryloxy groups, and ester groups; a sulfur atom in groups such as thiol groups, alkyl and aryl sulfide groups, sulfone groups, sulfonyl groups, and sulfoxide groups; a nitrogen atom in groups such as amines, amides, alkylamines, dialkylamines, arylamines, alkylarylamines, diarylamines, N-oxides, imides, and enamines; a silicon atom in groups such as trialkylsilyl groups, dialkylarylsilyl groups, alkyldiarylsilyl groups, and triarylsilyl groups; and other heteroatoms in various other groups.

Specific examples of substituents contemplated by the present invention include, without limitation, halogen, —OH, —NH₂, —NO₂, —CN, —C(O)OH, —C(S)OH, —C(O)NH₂, —C(S)NH₂, —S(O)₂NH₂, —NHC(O)NH₂, —NHC(S)NH₂, —NHS(O)₂NH₂, —C(NH)NH₂, —OR, —SR, —OC(O)R, —OC(S)R, —C(O)R, —C(S)R, —C(O)OR, —C(S)OR, —S(O)R, —S(O)₂R, —C(O)NHR, —C(S)NHR, —C(O)NRR, —C(S)NRR, —S(O)₂NHR, —S(O)₂NRR, —C(NH)NHR, —C(NH)NRR, —NHC(O)R, —NHC(S)R, —NRC(O)R, —NRC(S)R, —NHS(O)₂R, —NRS(O)₂R, —NHC(O)NHR, —NHC(S)NHR, —NRC(O)NH₂, —NRC(S)NH₂, —NRC(O)NHR, —NRC(S)NHR, —NHC(O)NRR, —NHC(S)NRR, —NRC(O)NRR, —NRC(S)NRR, —NHS(O)₂NHR, —NRS(O)₂NH₂, —NRS(O)₂NHR, —NHS(O)₂NRR, —NRS(O)₂NRR, —NHR, —NRR, where R at each occurrence is independently H, optionally substituted alkyl, optionally substituted aryl, or optionally substituted heteroaryl. Also contemplated is substitution with an optionally substituted hydrocarbyl moiety containing one or more of the following chemical functionalities: —O—, —S—, —NR—, —O—C(O)—, —O—C(O)—O—, —O—C(O)—NR—, —NR—C(O)—, —NR—C(O)—O—, —NR—C(O)—NR—, —S—C(O)—, —S—C(O)—O—, —S—C(O)—NR—, —S(O)—, —S(O)₂—, —O—S(O)₂—, —O—S(O)₂—O, —O—S(O)₂—NR—, —O—S(O)—, —O—S(O)—O—, —O—S(O)—NR—, —O—NR—C(O)—, —O—NR—C(O)—O—, —O—NR—C(O)—NR—, —NR—O—C(O)—, —NR—O—C(O)—O—, —NR—O—C(O)—NR—, —O—NR—C(S)—, —O—NR—C(S)—O—, —O—NR—C(S)—NR—, —NR—O—C(S)—, —NR—O—C(S)—O—, —NR—O—C(S)—NR—, —O—C(S)—, —O—C(S)—O—, —O—C(S)—NR—, —NR—C(S)—, —NR—C(S)—O—, —NR—C(S)—NR—, —S—S(O)₂—, —S—S(O)₂—O—, —S—S(O)₂—NR—, —NR—O—S(O)—, —NR—O—S(O)—O—, —NR—O—S(O)—NR—, —NR—O—S(O)₂—, —NR—O—S(O)₂—O—, —NR—O—S(O)₂—NR—, —O—NR—S(O)—, —O—NR—S(O)—O—, —O—NR—S(O)—NR—, —O—NR—S(O)₂—O—, —O—NR—S(O)₂—NR—, —O—NR—S(O)₂—, —O—P(O)R₂—, —S—P(O)R₂—, or —NR—P(O)R₂—, where R is at each occurrence is independently H, optionally substituted alkyl, optionally substituted aryl, or optionally substituted heteroaryl.

Each of the terms “halogen,” “halide,” and “halo” refers to —F, —Cl, —Br, or —I.

The term “aryl,” alone or in combination refers to a monocyclic or bicyclic ring system containing aromatic hydrocarbons such as phenyl or naphthyl, which may be optionally fused with a cycloalkyl of preferably 5-7, more preferably 5-6, ring members.

A “substituted aryl” is an aryl that is independently substituted with one or more, preferably 1, 2, 3, 4 or 5, also 1, 2, or 3 substituents, also 1 substituent, attached at any available atom to produce a stable compound, wherein the substituents are as described herein.

“Arylene” denotes divalent aryl, and “substituted arylene” refers to divalent substituted aryl.

“Heteroaryl” alone or in combination refers to a monocyclic aromatic ring structure containing 5 or 6 ring atoms, or a bicyclic aromatic group having 8 to 10 atoms, containing one or more, preferably 1-4, more preferably 1-3, even more preferably 1-2, heteroatoms independently selected from the group consisting of O, S, and N. Heteroaryl is also intended to include oxidized S or N, such as sulfinyl, sulfonyl and N-oxide of a tertiary ring nitrogen. A carbon or heteroatom is the point of attachment of the heteroaryl ring structure such that a stable compound is produced. Examples of heteroaryl groups include, but are not limited to, pyridinyl, pyridazinyl, pyrazinyl, quinaoxalyl, indolizinyl, benzo[b]thienyl, quinazolinyl, purinyl, indolyl, quinolinyl, pyrimidinyl, pyrrolyl, pyrazolyl, oxazolyl, thiazolyl, thienyl, isoxazolyl, oxathiadiazolyl, isothiazolyl, tetrazolyl, imidazolyl, triazolyl, furanyl, benzofuryl, and indolyl.

A “substituted heteroaryl” is a heteroaryl that is independently substituted, unless indicated otherwise, with one or more, preferably 1, 2, 3, 4 or 5, also 1, 2, or 3 substituents, attached at any available atom to produce a stable compound, wherein the substituents are as described herein.

The term “heteroarylene” refers to divalent heteroaryl, and “substituted heteroarylene” refers to divalent substituted heteroaryl.

“Heterocycloalkyl” means a saturated or unsaturated non-aromatic cycloalkyl group having from 5 to 10 atoms in which from 1 to 3 carbon atoms in the ring are replaced by heteroatoms of O, S or N, and are optionally fused with benzo or heteroaryl of 5-6 ring members, and includes oxidized S or N, such as sulfinyl, sulfonyl and N-oxide of a tertiary ring nitrogen. The point of attachment of the heterocycloalkyl ring is at a carbon or heteroatom such that a stable ring is retained. Examples of heterocycloalkyl groups include without limitation morpholino, tetrahydrofuranyl, dihydropyridinyl, piperidinyl, pyrrolidinyl, piperazinyl, dihydrobenzofuryl, and dihydroindolyl.

In this description, “optionally substituted heterocycloalkyl” denotes heterocycloalkyl or heterocycloalkyl that is substituted with 1 to 3 substituents, e.g., 1, 2 or 3 substituents, attached at any available atom to produce a stable compound, wherein the substituents are as described herein.

The term “heteroalkyl” means a saturated or unsaturated alkyl group having from 1 to about 20 carbon atoms, preferably 1 to 10 carbon atoms, more preferably 1 to 6 carbon atoms, even more preferably 1 to 3 carbon atoms, in which from 1 to 3 carbon atoms are replaced by heteroatoms of O, S or N. Heteroalkyl is also intended to include oxidized S or N, such as sulfinyl, sulfonyl and N-oxide of a tertiary ring nitrogen. The point of attachment of the heteroalkyl substituent is at an atom such that a stable compound is formed. Examples of heteroalkyl groups include, but are not limited to, N-alkylaminoalkyl (e.g., CH₃NHCH₂—), N,N-dialkylaminoalkyl (e.g., (CH₃)₂NCH₂—), and the like.

In general, tubulysins of the present invention may be synthesized by methods of classical solution synthesis. The identity and purity of the compounds of the invention may be verified using any of a variety of analytical techniques available to one skilled in the art such as ¹H-NMR, analytical HPLC, LC-MS, and/or matrix-assisted laser-desorption ionization time-of-flight mass spectrometry (MALDI-TOF MS-monoisotopic).

A wide array of organic synthetic techniques exist in the art to meet the challenge of constructing tubulysins. Many such organic synthetic methods are described in detail in standard reference sources utilized by those skilled in the art. (e.g., March, 1994, Advanced Organic Chemistry, Reactions, Mechanisms and Structure, New York, McGraw Hill). Thus, the techniques useful to synthesize reagents and tubulysins of the present invention are readily available to those skilled in the art of organic chemical synthesis. In particular, the term “Grignard conditions” refers to chemical conditions under which the Grignard reaction (i.e., an organometallic chemical reaction involving reaction of alkyl- or aryl-magnesium halides (i.e., Grignard reagents) with electrophiles) proceeds.

The methodology of the present invention contemplates the coupling of amino acids, and optionally substituted derivatives thereof, to form amide linkage. Methods for the synthesis of compounds of the invention employing protection (i.e., protected amino acid), deprotection, activation and coupling of reagents are well known in the art.

The phrase “amino acid” here refers to an organic molecule containing both an amino group (NH₂) and a carboxylic acid group (COOH), either of which group may form an amide bond with an adjacent amino acid or other functional group capable of forming an amide bond. Amino acids include the physiologic amino acids, including alanine, cysteine, aspartic acid, glutamic acid, phenylalanine, glycine, histidine, isoleucine, lysine, leucine, methionine, asparagine, proline, glutamine, arginine, serine, threonine, valine, tryptophan, and tyrosine.

As used herein, “optionally substituted amino acid derivatives” and terminology of like content refer to amino acids containing a substitution, on the side chain moiety or on the backbone nitrogen, as described herein.

The phrase “amino protecting group” and like refer to standard moieties used to protect amines, e.g. during synthesis employing amine containing reagent. Amine containing reagents can be protected at the amine nitrogen with a variety of standard amino protecting groups, such as t-butoxycarbonyl (Boc), 9-fluorenylmethoxycarbonyl (Fmoc), Alloc (allyl carbamate), Troc (trichloroethyl carbamate), or Cbz (benzylcarboxy carbamate), for instance. Methods for protection by and deprotection (i.e., removal of a protecting group) of amino protecting groups are well known in the art.

As used here, “acid protecting group” refers to standard moieties used to protect acid functionalities, e.g. during synthesis employing carboxylic acid containing reagent. Carboxylic containing reagents can be protected with a variety of standard protecting groups such as in tBu (tert-butyl ester), Bn (benzyl ester), Allyl (allyl ester), Pfp (pentafluorophenyl ester), Me (methyl ester), Pmb (p-methoxybenzyl ester), Mem (methoxyethoxymethyl ester). Methods for protection by and deprotection of acid protecting groups are well known in the art. Methods for protection and deprotection (i.e., removal of a protecting group) are well known in the art.

In general, the protection of functional groups by protecting groups as described herein, the protecting groups themselves, and the reactions and conditions required to remove such protective groups (i.e., conditions for deprotection) are described in standard reference books, e.g., for peptide synthesis, and in special references on protective groups such as J. F. W. McOmie, “Protective Groups in Organic Chemistry,” Plenum Press, London and New York 1973, in “Methoden der organischen Chemie” (Methods of organic chemistry), Houben-Weyl, 4th edition, Volume 15/1, Georg Thieme Verlag, Stuttgart 1974, and in T. W. Greene, “Protective Groups in Organic Synthesis,” Wiley, N.Y.

“Activation” denotes the standard use of conventional moieties to activate carboxyl groups, e.g., via carboxyl activating agents. Reagents comprising a carboxyl group substituent may be activated by a variety of standard activating agents, such as N,N′-dicyclohexylcarbodiimide (DCC), N,N′-diisopropylcarbodiimide (DIC) or O-benzotriazol-1-yl-N,N,N′,N′-tetramethyluronium-hexafluorophosphate (HBTU), with or without 4-dimethylaminopyridine (DMAP), 1-hydroxybenzotriazole (HOBT), benzotriazol-1-yloxy-tris(dimethylamino)phosphonium-hexafluorophosphate (BOP), bis(2-oxo-3-oxazolidinyl) phosphine chloride (BOPCl), DEPBT (3-(Diethoxy-phosphoryloxy)-3H-benzo[d][123]triazin-4-one), BEP (2-bromo-1-ethyl pyridinium tetrafluoroborate), HATU (N,N,N′,N′-tetramethyl-O-(7-azabenzotriazol-1-yl)uronium hexafluorophosphate), TBTU (N,N,N′N′-tetramethyl-O-(benzotriazol-1-yl)uronium tetrafluoroborate), PyBop (benzotriazol-1-yloxy)tripyrrolidinophosphonium hexafluorophosphate), and the like.

The phrase “pharmaceutically acceptable salt” refers to a salt that retains the biological effectiveness of the free acids and bases of the specified compound and that is not biologically or otherwise unacceptable. A compound of the invention may possess a sufficiently acidic, a sufficiently basic, or both functional groups, and accordingly react with any of a number of inorganic or organic bases, and inorganic and organic acids, to form a pharmaceutically acceptable salt. Exemplary pharmaceutically acceptable salts include those salts prepared by reaction of the compounds of the present invention with a mineral or organic acid or an inorganic base, such as salts including sodium, chloride, sulfates, pyrosulfates, bisulfates, sulfites, bisulfites, phosphates, monohydrogenphosphates, dihydrogenphosphates, metaphosphates, pyrophosphates, chlorides, bromides, iodides, acetates, substituted acetates, propionates, decanoates, caprylates, acrylates, formates, isobutyrates, caproates, heptanoates, propiolates, oxalates, malonates, succinates, suberates, sebacates, fumarates, maleates, butyne-1,4 dioates, hexyne-1,6-dioates, benzoates, chlorobenzoates, methylbenzoates, dinitrobenzoates, hydroxybenzoates, methoxybenzoates, phthalates, sulfonates, xylenesulfonates, phenylacetates, phenylpropionates, phenylbutyrates, citrates, lactates, gamma.-hydroxybutyrates, glycollates, tartrates, methane-sulfonates, propanesulfonates, naphthalene-1-sulfonates, naphthalene-2-sulfonates, and mandelates.

“Pharmaceutically acceptable” characterizes a compound or formulation that does not have properties that would cause a reasonably prudent medical practitioner to avoid administration of the material to a patient, taking into consideration the disease or conditions to be treated and the respective route of administration.

In some embodiments of the process for the preparation of compounds with structure of Formula I (supra), R¹ is H, and the C-terminal amino acid is tubuphenylalanine. In further embodiments, R¹ is OH, and the C-terminal amino acid is tubutyrosine.

Further this aspect, in some embodiments R² is H. In some embodiments, R² is C(O)R⁶, wherein R⁶ is H, optionally substituted alkyl, optionally substituted aryl, or optionally substituted heteroaryl. In some embodiments, R⁶ is alkyl, for example without limitation, C₁₋₁₈ alkyl, C₁₋₁₂ alkyl, C₁₋₁₀ alkyl, C₁₋₆ alkyl, C₁₋₃ alkyl, C₁₋₂ alkyl, or C₁ alkyl. In some embodiments, R⁶ is unsubstituted methyl. In some embodiments, R⁶ is substituted C₁-C₁₈ alkyl, C₁₋₁₂ alkyl, C₁₋₁₀ alkyl, C₁-6 alkyl, C₁₋₃ alkyl, C₁₋₂ alkyl, or C₁ alkyl, with substitution as described herein. In some embodiments, R⁶ is aryl, for example without limitation, phenyl or naphthyl. In some embodiments, R⁶ is substituted aryl, for example without limitation, chlorophenyl, nitrophenyl, toluenyl, or other substituted aryl. In some embodiments, R⁶ is optionally substituted heteroaryl, for example without limitation, pyridinyl, pyridazinyl, pyrazinyl, quinaoxalyl, indolizinyl, benzo[b]thienyl, quinazolinyl, purinyl, indolyl, quinolinyl, pyrimidinyl, pyrrolyl, pyrazolyl, oxazolyl, thiazolyl, thienyl, isoxazolyl, oxathiadiazolyl, isothiazolyl, tetrazolyl, imidazolyl, triazolyl, furanyl, benzofuryl, indolyl, or derivatives thereof substituted as described herein.

Further this aspect, in some embodiments R³ is C₁₋₆ alkyl, for example without limitation, methyl, ethyl, n-propyl, prop-2-yl, n-butyl, but-2-yl, or tert-butyl. In some embodiments, R³ is C₁₋₃ alkyl. In some embodiments, R³ is prop-2-yl.

In further embodiments of this aspect, R⁴ is an amino acid selected from the group consisting of glycine, cysteine, alanine, histidine, asparagine, glutamine, arginine, threonine, valine, leucine, isoleucine, phenylalanine, tryptophan, serine, lysine, aspartic acid, methionine, glutamic acid, tyrosine, and optionally substituted derivatives thereof. In some embodiments, R⁴ is an amino acid with defined stereochemistry. In some embodiments, R⁴ is of L-configuration. In some embodiments, R⁴ is of D-configuration. In some embodiments, R⁴ is L-isoleucine.

In further embodiments of this aspect, R⁵ is optionally substituted heteroalkyl, e.g., N,N-dimethylaminomethyl, and the like. In some embodiments, R⁵—COOH is R⁷R⁸N—(CH₂)ₙ—COOH, wherein each of R⁷ and R⁸ are independently C₁₋₆ alkyl, or R⁷ and R⁸, together with the nitrogen to which they are attached, form an optionally substituted 5-7 membered heterocycloalkyl or heteroaryl, and n is 1 to 6. In some embodiments, R⁵—COOH is 2-(dimethylamino)acetic acid.

In further embodiments of this aspect, R⁵ is optionally substituted heterocycloalkyl. In some embodiments, R⁵—COOH is 1-methylpiperidine-2-carboxylic acid (Mep).

In some embodiments of the process for the preparation of a compound with structure of Formula I, the second protected amino is of defined stereochemistry.

In some embodiments, the second protected amino acid has the structure of Formula IIIₐ

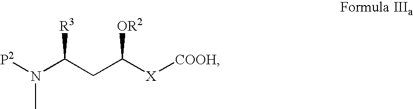

the first compound has the structure of Formula IVₐ

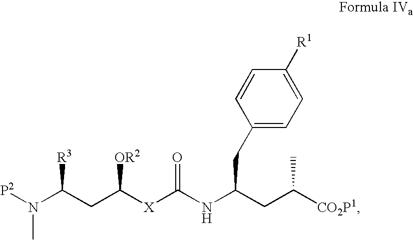

the second compound has the structure of Formula Vₐ

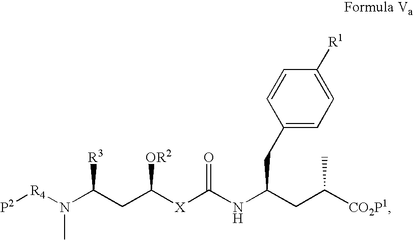

the reagent with structure of Formula VI is 1-methylpiperidine-2-carboxylic acid or acid protected derivative thereof, and the compound with structure of Formula I has the structure of Formula Iₐ

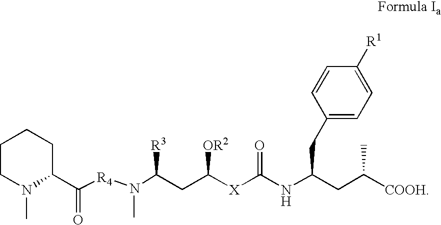

In further embodiments of this aspect, the second protected amino acid has the structure of Formula III_{b}:

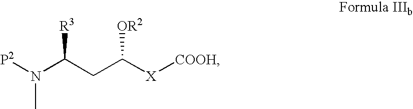

the first compound has the structure of Formula IV_{b}:

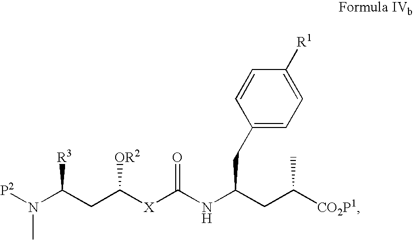

the second compound has the structure of Formula V_{b}:

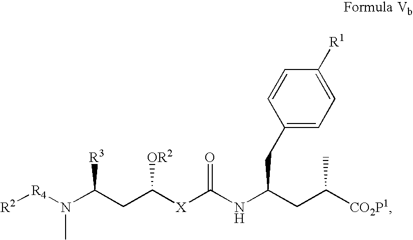

the reagent with structure of Formula VI is 1-methylpiperidine-2-carboxylic acid or acid protected derivative thereof, and the compound with structure of Formula I has the structure of Formula I_{b}

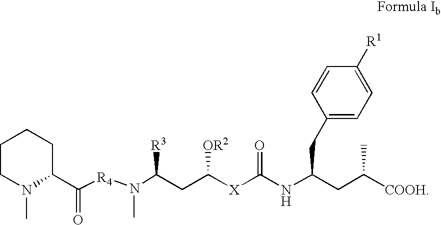

With respect to preparing a compound with structure of Formula I, some embodiments of the inventive process involve fluorenylmethoxycarbonyl-isoleucyl-fluoride as the third protected amino acid.

Further this aspect, in some embodiments, P¹ is an acid protecting group as described herein. In some embodiments, P¹ is allyl.

Further this aspect, in some embodiments, P² and P³ of step (a) are amino protecting groups as described herein. In some embodiments, P² and P³ of step (a) are independently t-butyloxycarbonyl. In some embodiments, P² of step (b) is fluorenylmethoxycarbonyl.

Further this aspect, in some embodiments the reagent with structure of Formula VI of step (c) is protected prior to reaction with an acid protecting group as described herein. In some embodiments, the acid protected derivative of the reagent with structure of Formula VI is Mep-pentafluorophenyl ester.

Further this aspect, in some embodiments X is heteroarylene or heteroarylene substituted as described herein. In some embodiments, X is a five-membered heteroarylene. In some embodiments, X contains N and S. In some embodiments, X is thiazole-diyl. In some embodiments, when X is thiazole-diyl, the second protected amino acid has the structure of Formula VIIₐ:

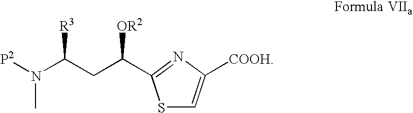

Further this aspect, in some embodiments, when X is thiazole-diyl, the second protected amino acid has the structure of Formula VIIIₐ:

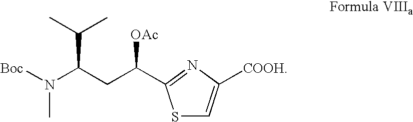

Further this aspect, in some embodiments, when X is thiazole-diyl, the second protected amino acid has the structure of Formula VII_{b}:

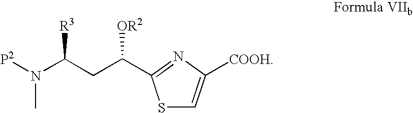

Further this aspect, in some embodiments, when X is thiazole-diyl, the second protected amino acid has the structure of Formula VIII_{b}:

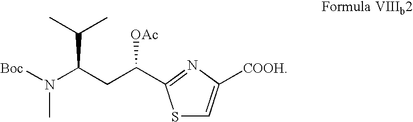

In some embodiments of the process for the preparation of N¹⁴-desacetoxytubulysin H, the second protected amino acid has the structure of Formula IXhₐ

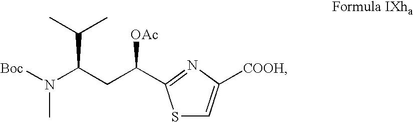

the first compound has the structure of Formula XIIIₐ

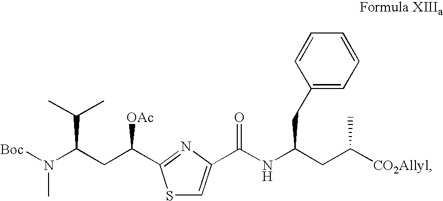

the second compound has the structure of Formula XIVₐ

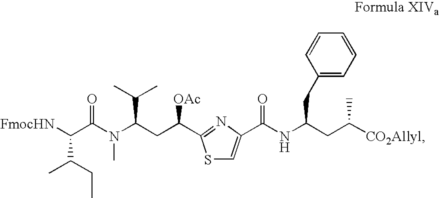

and the N¹⁴-desacetoxytubulysin H has the structure of Formula Xₐ

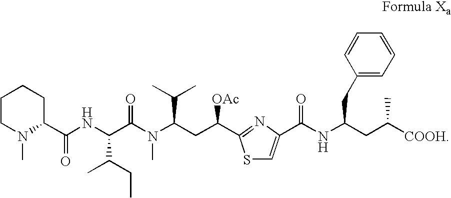

Further this aspect, in some embodiments the second protected amino acid has the structure of Formula IXh_{b}

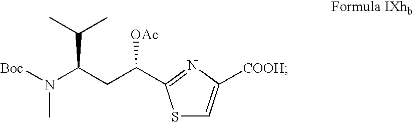

the first compound has the structure of Formula XIII_{b}:

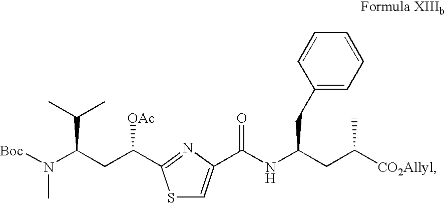

the second compound has the structure of Formula XIV_{b}

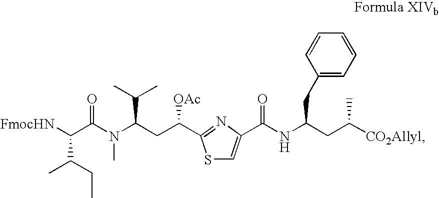

and the N¹⁴-desacetoxytubulysin H has the structure of Formula X_{b}

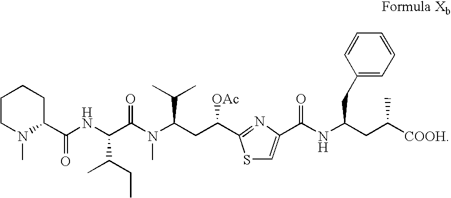

In some embodiments for the process for the preparation of a compound with structure of Formula XV, the second protected amino acid has the structure of Formula IXhₐ:

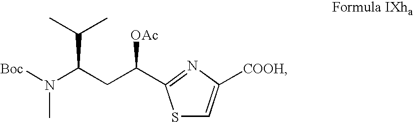

the first compound has the structure of Formula XIIIₐ:

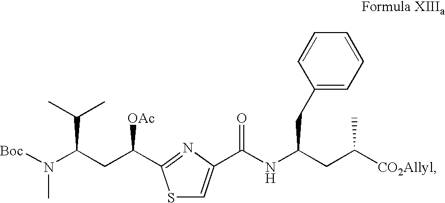

the second compound has the structure of Formula XIVₐ

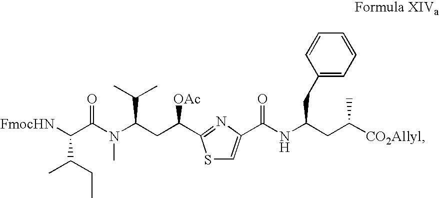

and the compound having the structure of Formula XIV has the structure of Formula XVₐ

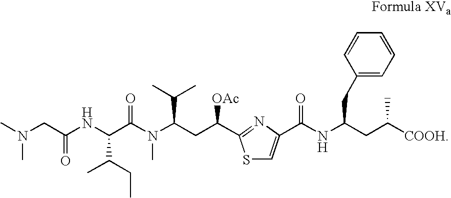

Further this aspect, in some embodiments, the second protected amino acid has the structure of Formula IXh_{b}:

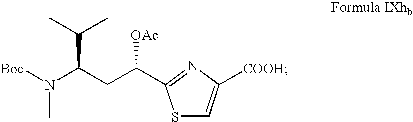

the first compound has the structure of Formula XIII_{b}

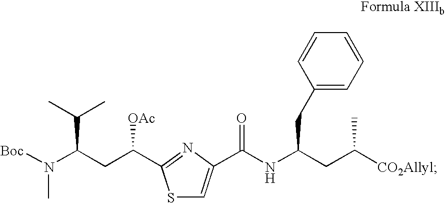

said second compound has the structure of Formula XIV_{b}

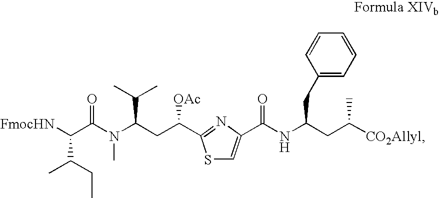

and the compound having the structure of Formula XIV has the structure of Formula XV_{b}

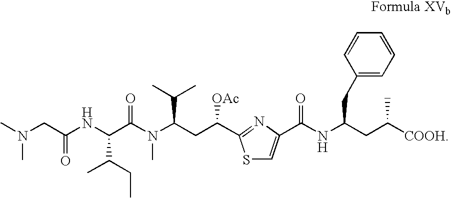

In some embodiments for the method of inhibiting proliferation of a cell, the method includes contacting a cell with a compound or pharmaceutically acceptable salt thereof as described herein. As discussed in detail here, compounds of the invention can be inhibitors of the proliferation of a human cancer cell line. Thus, the terms “inhibitor,” “inhibiting” and the like, in the context of cell proliferation, refer to an effect of diminishing growth of a cell or cell culture.

Without wishing to be bound by theory, the inventors note in this regard that the antiproliferative activity of tubulysins is thought to result from the depolymerization of microtubles and/or the induction of mitotic arrest. Additionally, tubulysins can induce apoptosis in cancers cells, but not in normal cells (Kaur et al., supra), and show significant potential antiangiogenic properties in in vitro assays. Accordingly, contacting cells with a compound of the invention or a pharmaceutically acceptable salt thereof can result in inhibition of proliferation of the cell. The measurement of cell viability and growth can be performed by a variety of conventional methodologies, including without limitation the following assays in cell culture: Trypan blue staining, measuring the uptake of radioactive substances, usually tritium-labeled thymidine, and the reduction of tetrazolium salts (MTT assay).

In some embodiments relating to screening for an inhibitor of cell proliferation, the inventive methodology includes contacting a cell with a test compound, which compound is as described herein, determining the effect of the test compound on cell proliferation, and comparing the effect thus determined with a control cell tested in the absence of a test compound. Metrics for determining the extent of inhibition resulting from the activity of a test compound are routinely employed in the art, including for example without limitation IC₅₀ (i.e., 50% inhibitor concentration), and GI₅₀ (i.e., 50% growth inhibiting concentration). Thus, screening for an inhibitor of cell proliferation entails calculation of an inhibitor concentration that provides a defined level of inhibition (e.g., GI₅₀) for a plurality of test compounds, and comparing the resulting concentrations to ascertain whether a test compound inhibits cell proliferation.

### EXAMPLE 1

### Chemical Synthetic and Analytic Methods

All reactions involving moisture sensitive reagents were conducted in oven-dried glassware under a nitrogen or argon atmosphere. Anhydrous solvents were obtained through standard laboratory protocols. Analytical thin-layer chromatography (TLC) was preformed on SiO2 60 F-254 plates available from Merck. Visualization was accomplished by UV irradiation at 254 nm, or by staining with any one of the following reagents: iodine, 5% phosphomolybdic acid hydrate in ethanol, ninhydrin (0.3% w/v in glacial acetic acid/n-butyl alcohol 3:97), Vaughn's reagent (4.8 g of (NH₄)₆Mo₇O₂₄.4H₂O and 0.2 g of Ce(SO₄)₂.4H₂O in 10 mL of conc. H₂SO₄ and 90 mL of H₂O), or para-anisaldehyde (7.5 mL of para-anisaldehyde, 25 mL of conc. H2SO4, and 7.5 mL of acetic acid in 675 mL of 95% ethanol). Flash column chromatography was performed using SiO2 60 (particle size 0.040-0.055 mm, 230-400 mesh, EM science distributed by Fisher Scientific). Melting points were obtained on a Meltemp II™ capillary melting point apparatus fitted with a Fluke 51™ digital thermometer and are not corrected. Specific rotations of chiral compounds were obtained at the designated concentration and temperature on a Perkin Elmer 241 polarimeter using a 1 dm cell. Infrared spectra were collected on a Nicolet Avatar™ 360 FT-IR spectrometer from thin films deposited onto NaCl plates. Proton and carbon NMR spectra were obtained on Bruker Avance™ 300 and 500 MHz NMR spectrometers. Chemical shifts are reported as δ values in parts per million (ppm) as referenced to residual solvent. ¹H NMR spectra are tabulated as follows: chemical shift, multiplicity (s=singlet, bs=broad singlet, d=doublet, t=triplet, q=quartet, m=multiplet), number of protons, and coupling constant(s). Mass spectra were obtained at the University of Pittsburgh Mass Spectrometry facility. A Varian HPLC system equipped with Gilson 215 Liquid Handler and fraction collector was used for preparative HPLC purification. A Varian Dynamax Microsorb C18 column (250 mm×10 mm, or 250 mm×21.4 mm, 60 Å) was used. LC-MS analysis was performed on an Agilent 1100 instrument, using an analytical C18 column (Waters Xterra MS 100×4.6 mm, 3.5 μm, 0.4 mL/min).

### EXAMPLE 2

### Synthesis of (R)-Methyl 3-(benzyloxycarbonylamino)-4-methylpentanoate ([[1,4]])¹

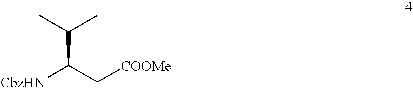

To a solution of Cbz-Val-OH (1.0 g, 4.1 mmol) and triethylamine (0.60 mL, 4.3 mmol) in anhydrous THF (15 mL) cooled to −20° C. was added isobutyl chloroformate (0.66 mL, 5.1 mmol) dropwise over 5 min; the resulting white suspension was stirred further for 30 min. A diazomethane solution (˜16.9 mmol) in ether (50 mL), which was prepared from diazald (5.1 g, 24.0 mmol) using an Aldrich MiniDiazald apparatus and dried over potassium hydroxide (pellet) prior to use, was then introduced into the reaction mixture via cannula. The mixture was stirred further overnight, allowing the temperature to gradually rise to room temperature. Acetic acid was then added dropwise until there was no effervescence, and the mixture was diluted in ether (50 mL), washed with saturated sodium bicarbonate (30 mL) and brine (30 mL), dried with sodium sulfate, concentrated, and chromatographed (Et₂O/hexanes, 1:3) to give a yellow solid as the diazoketone (0.96 g, 84%). ¹H NMR (CDCl₃, 300 MHz) δ 0.89-0.91 (d, 3H, J=6.8 Hz), 0.99-1.01 (d, 3H, J=6.8 Hz), 2.07-2.14 (m, 1H), 4.14-4.15 (m, 1H), 5.11 (s, 2H), 5.38-5.41 (m, 2H), 7.35-7.38 (m, 5H). ¹³C NMR (CDCl₃, 75 MHz) δ 17.2, 19.3, 31.0, 54.6, 62.8, 67.0, 128.0, 128.1, 128.4, 136.2, 156.3, 193.2. IR (KBr, cm⁻¹) 1232, 1366, 1525, 1632, 1713, 2107, 2965, 3324.

To a solution of the diazoketone (0.91 g, 3.3 mmol) in anhydrous methanol (15 mL) cooled at −35° C. was added a solution of silver benzoate (80 mg, 0.35 mmol) in freshly distilled triethylamine (over CaH₂) (1 mL). The reaction bottle was wrapped with aluminum foil to keep it from light, and the mixture was stirred overnight, during which time it gradually rose to room temperature. The solvent was evaporated, and the residue was taken up in ethyl acetate (60 mL), washed with saturated sodium bicarbonate (30 mL) and brine (30 mL), dried with sodium sulfate, concentrated, and chromatographed (Et₂O/hexanes, 1:3) to give a white solid (0.77 g, 65% for two steps); mp 44.5-45.5° C.; [α]²³_{D}−22.7 (c 2.2, CH₂Cl₂). ¹H NMR (CDCl₃, 300 MHz) δ 0.92-0.94 (d, 6H, J=6.8 Hz), 1.79-1.88 (m, 1H), 2.52-2.54 (m, 2H), 3.66 (s, 3H), 3.81-3.86 (m, 1H), 5.10 (s, 2H), 5.15-5.18 (m, 1H), 7.36-7.37 (m, 5 H). ¹³C NMR (CDCl₃, 75 MHz) δ 18.5, 19.3, 31.6, 36.8, 51.7, 53.6, 66.6, 128.0, 128.5, 136.5, 156.0, 172.2. IR (film, cm⁻¹) 1239, 1531, 1731, 2961, 3338. HRMS (EI) calcd for C₁₅H₂₁NO₄ (M⁺) 279.1471. found 279.1478.

### EXAMPLE 3

### Synthesis of (R)-Benzyl 1-(tert-butyldimethylsilyloxy)-4-methylpentan-3-ylcarbamate (5)²

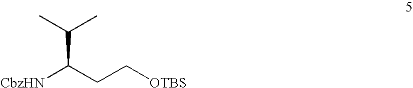

To an ice-cooled solution of 4 (0.66 g, 2.4 mmol) in anhydrous THF (10 mL) was added dropwise lithium borohydride (2.0 M solution in THF, 1.8 mL, 3.6 mmol) over 5 min. A solution of methanol (0.20 mL, 4.8 mmol) in anhydrous THF (5 mL) was then added over 10 min, and the mixture was stirred further overnight, allowing the temperature to rise to room temperature. The solvent was evaporated, and the residue was dissolved in ethyl acetate (70 mL), washed with hydrochloric acid (1 N, 20 mL×2), saturated sodium bicarbonate (20 mL), and brine (20 mL), dried with sodium sulfate, concentrated, and chromatographed (EtOAc/hexanes, 1:1) to give a white solid (0.42 g, 71%) as the desired alcohol; mp 52.0-53.0° C.; [α]²³_{D}+12.1 (c 4.0, CH₂Cl₂). ¹H NMR (CDCl₃, 300 MHz) δ 0.92-0.94 (d, 3H, J=6.9 Hz), 0.95-0.97 (d, 3H, J=6.8 Hz), 1.27-1.40 (m, 1H), 1.70-1.88 (m, 2H), 3.02 (bs, 1H), 3.58-3.73 (m, 3H), 4.63-4.66 (d, 1H, J=9.1 Hz), 5.07-5.18 (d_{AB}, 2H, J=11.6 Hz), 7.36-7.38 (m, 5H). ¹³C NMR (CDCl₃, 75 MHz) □ 17.9, 19.1, 32.0, 35.2, 52.9, 58.9, 66.7, 127.9, 128.0, 128.4, 136.3, 157.4. IR (film, cm⁻¹) 1048, 1251, 1537, 1693, 2875, 2959, 3324.

To a solution of the alcohol (0.34 g, 1.4 mmol) and imidazole (0.18 g, 2.4 mmol) in anhydrous DMF (2 mL) was added a solution of tert-butyldimethylchlorosilane (0.32 g, 2.1 mmol) in anhydrous THF (2 mL). The mixture was stirred further overnight, diluted in water (10 mL), and extracted with ether (20 mL×3). The combined organics were washed with hydrochloric acid (1 N, 20 mL), saturated sodium bicarbonate (20 mL), and brine (20 mL), dried with sodium sulfate, concentrated, and chromatographed (Et₂O/hexanes, 1:7) to give a colorless oil (0.49 g, 99%). [α]²³_{D}+6.6 (c 5.0, CH₂Cl₂). ¹H NMR (CDCl₃, 300 MHz) δ 0.05-0.06 (2 s, 6 H), 0.90 (s, 9H), 0.93 (m, 6H), 1.49-1.61 (m, 1H), 1.72-1.89 (m, 2H), 3.60-3.74 (m, 3H), 4.99-5.02 (d, 1H, J=9.4 Hz), 5.05-5.14 (d_{AB}, 2H, J=12.5 Hz), 7.35-7.37 (m, 5H). ¹³C NMR (CDCl₃, 75 MHz) δ −5.5, 18.0, 18.2, 18.8, 25.6, 25.8, 31.8, 34.5, 54.2, 60.6, 66.3, 127.8, 128.4, 136.8, 156.2. IR (film, cm⁻¹) 776, 836, 1096, 1255, 1537, 1699, 2858, 2958, 3333. m/z (APCI) 366 ([M+H]⁺).

### EXAMPLE 4

### Synthesis of (R)-Benzyl 1-hydroxy-4-methylpentan-3-yl(methyl)carbamate (6)

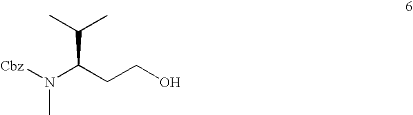

To an ice-cooled solution of 5 (0.40 g, 1.1 mmol) in anhydrous THF (2 mL) was added a solution of NaHMDS (0.28 g, 1.5 mmol) in THF (1 mL), and the mixture was stirred further for 20 min before iodomethane (0.1 mL, 1.6 mmol) was added. The mixture was stirred further overnight, during which time it gradually rose to room temperature. The mixture was diluted in ethyl acetate (60 mL), washed with hydrochloric acid (1 N, 10 mL) and brine (10 mL), dried with sodium sulfate, evaporated, and chromatographed (Et₂O/hexanes, 1:8) to give a colorless oil (0.40 g, 96%) as the methylated product. [α]²³_{D}+5.2 (c 2.2, CH₂Cl₂). NMR analysis in CDCl₃ showed rotomers at room temperature. ¹H NMR (DMSO-d₆, 338 K, 300 MHz) δ 0.00 (s, 6H), 0.76-0.78 (d, 3H, J=6.6 Hz), 0.85 (s, 9H), 0.88-0.90 (d, 3H, J=6.6 Hz), 1.62-1.80 (m, 3H), 2.70 (s, 3H), 3.49-3.51 (m, 2H), 3.65-3.72 (dt, 1H, J=10.0, 3.9 Hz), 5.08 (s, 2H), 7.33-7.34 (m, 5H). ¹³C NMR (DMSO-d₆, 338 K, 75 MHz) δ −5.9, 17.4, 19.1, 19.5, 25.4, 28.8, 29.7, 31.9, 58.7, 59.8, 65.6, 126.8, 127.2, 127.8, 137.3, 155.6. IR (film, cm⁻¹) 835, 1098, 1137, 1252, 1471, 1701, 2857, 2957. HRMS (EI) calcd for C₂₁H₃₇NO₃Si (M⁺) 379.2543. found 379.2536.

To a solution of the above methylated product (0.11 g, 0.28 mmol) in THF (2 mL) was added tetrabutylammonium fluoride (1.0 M solution in THF, 0.34 mL, 0.34 mmol), and the mixture was stirred at room temperature for 4 h. The mixture was diluted in ethyl acetate (40 mL), washed with brine (10 mL), dried with sodium sulfate, concentrated, and chromatographed (EtOAc/hexanes, 1:1) to give a colorless oil (74 mg, 98%); [α]²³_{D}−13.1 (c 2.8, CH₂Cl₂). NMR analysis in CDCl₃ showed rotomers at room temperature. ¹H NMR (DMSO-d₆, 333 K, 300 MHz) δ 0.77-0.78 (d, 3H, J=6.5 Hz), 0.88-0.90 (d, 3H, J=6.3 Hz), 1.55-1.77 (m, 3H), 2.70 (s, 3H), 3.25-3.47 (m, 2H), 3.64-3.72 (dt, 1H, J=10.2, 3.5 Hz), 4.10 (bs, 1H), 5.08 (s, 2H), 7.30-7.35 (m, 5H). ¹³C NMR (DMSO-d₆, 333 K, 75 MHz) δ 19.2, 19.6, 28.6, 29.7, 32.1, 58.2, 58.9, 65.6, 126.8, 127.2, 127.9, 137.1, 155.8. IR (film, cm⁻¹) 1138, 1455, 1682, 1694, 2875, 2961, 3466. HRMS (EI) calcd for C₁₅H₂₃NO₃ (M⁺) 265.1678. found 265.1677.

### EXAMPLE 5

### Synthesis of (R)-3-((tert-Butoxycarbonyl)methyl)amino-4-methylpentanal (7)

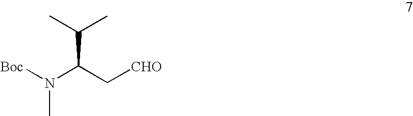

A mixture of 6 (0.73 g, 2.7 mmol), di-tert-butyldicarbonate (0.72 g, 3.2 mmol), and palladium on activated carbon (5% Pd, 78 mg) in methanol (15 mL) was stirred under a hydrogen balloon at room temperature overnight. The solvent was evaporated, and the residue was chromatographed (EtOAc/hexanes, 1:1) to give a colorless oil (0.58 g, 92%) as the Boc-protected amino alcohol; [α]²³_{D}−22.3 (c 3.2, CH₂Cl₂). For the major rotomer: ¹H NMR (CDCl₃, 300 MHz) δ 0.86-0.89 (d, 3H, J=6.5 Hz), 0.95-0.97 (d, 3H, J=6.5 Hz), 1.25-1.39 (m, 1H), 1.46 (s, 9H), 1.61-1.73 (m, 1H), 1.84-2.03 (m, 1H), 2.61 (s, 3H), 3.39 (m, 1H), 3.53-3.61 (m, 1H), 3.79-3.87 (m, 1H). ¹³C NMR (CDCl₃, 75 MHz) δ 20.1, 27.9, 28.4, 30.0, 31.8, 57.4, 58.9, 79.9, 157.9. IR (film, cm⁻¹) 1047, 1177, 1393, 1694, 2968, 3454. HRMS (EI) calcd for C₁₂H₂₅NO₃ (M⁺) 231.1834. found 231.1830.

To a mixture of Dess-Martin periodinane (1.19 g, 2.8 mmol) in anhydrous dichloromethane (6 mL) was added dropwise a solution of the above Boc-protected amino alcohol (0.57 g, 2.4 mmol) in dichloromethane (8 mL), and the mixture was stirred at room temperature for 2 h. The solvent was removed, and the residue was taken up in ether (70 mL), washed with a mixture of sodium hydroxide (1.0 N, 10 mL) and sodium thiosulfate (1.0 M, 10 mL), saturated sodium bicarbonate (10 mL), and brine (10 mL), dried with sodium sulfate, evaporated, and chromatographed (Et₂O/hexanes, 1:2) to give a colorless oil (0.50 g, 89%). [α]²³_{D}−75.9 (c 2.8, CH₂Cl₂). ¹H NMR (CDCl₃, 300 MHz) δ 0.88-0.91 (d, 3H, J=6.6 Hz), 0.93-0.97 (appar. t, 3H, J=6.2 Hz), 1.44-1.47 (2 s, 9H), 1.72-1.83 (m, 1H), 2.43-2.62 (m, 2H), 2.66-2.77 (2 s, 3H), 4.05-4.14 (dt, 1H, J=10.0, 4.8 Hz, rotomer 1), 4.28-4.36 (dt, 1H, J=10.6, 4.2 Hz, rotomer 2), 9.65-9.67 (m, 1H). ¹³C NMR (CDCl₃, 75 MHz) δ 19.2, 19.4, 20.0, 20.1, 28.3, 28.4, 28.9, 29.1, 30.4, 30.8, 44.9, 56.7, 56.8, 79.6, 80.0, 155.6, 156.1, 200.8, 201.6. IR (film, cm⁻¹) 1153, 1366, 1688, 1726, 2726, 2971. HRMS (EI) calcd for C₁₂H₂₃NO₃ (M⁺) 229.1678. found 229.1674. calcd for C₉H₁₆NO₃ ([M−C₃H₇]⁺) 186.1130. found 186.1130.

### EXAMPLE 6

### Synthesis of ethyl 2-bromothiazole-4-carboxylate

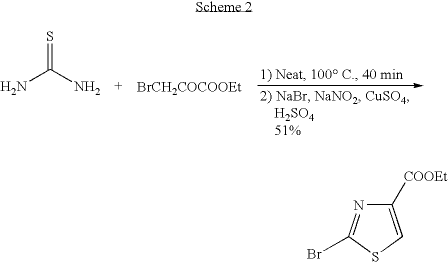

Following the procedures according to Kelly et al.³ (Scheme 2), a mixture of ethyl bromopyruvate (6.5 g, 28.3 mmol) and thiourea (2.2 g, 28.4 mmol) was heated slowly to 100° C. and maintained at that temperature for 40 min to afford a brown clear solution. Upon cooling to room temperature a yellow chunk was generated, which was dissolved in sulfuric acid (9 N, 250 mL) and transferred into a 500 mL three-necked bottle equipped with a mechanical stirrer, an addition funnel, and a gas outlet with an inverted wide-mouth funnel suspended just above a sodium hydroxide solution (4 N, 20 mL). The solution was cooled in an ice bath, and cupric sulfate pentahydrate (21.2 g, 84.9 mmol) and sodium bromide (11.6 g, 113.2 mmol) was added in portions. A solution of sodium nitrite (2.7 g, 45.9 mmol) in water (30 mL) was then added dropwise over 30 min in a well-ventilated hood. Stirring was continued for 4 h, during which time the bath temperature gradually rose to room temperature. The mixture was diluted in water (100 mL) and extracted with ether (100 mL×4). The combined organics were washed with saturated sodium bicarbonate (50 mL×2) and brine (50 mL), dried with sodium sulfate, evaporated, and chromatographed (EtOAc/hexanes, 1:8) to give a yellow solid (3.3 g, 51% for two steps); mp 68.2-69.5° C. (lit. 68.5-69.2° C.) See Kelly, T. R. & Lang, F., 1996, J. Org. Chem. 61:4263. ¹H NMR (CDCl₃, 300 MHz) δ 1.37-1.42 (t, 3H, J=7.1 Hz), 4.38-4.45 (q, 2H, J=7.1 Hz), 8.12 (s, 1H). ¹³C NMR (CDCl₃, 75 MHz) δ 14.2, 61.8, 130.8, 136.8, 147.2, 160.7. IR (KBr, cm⁻¹) 774, 1011, 1121, 1224, 1329, 1431, 1478, 1488, 1717, 2986, 3090. m/z (ESI) 258, 260 ([M+Na]⁺).

### EXAMPLE 7

### Synthesis of 2-Bromo-4-((tert-butyldimethylsilyloxy)methyl)thiazole (8)²

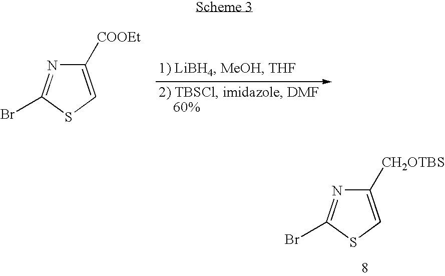

With reference to Scheme 3, to an ice-cooled solution of ethyl 2-bromothiazole-4-carboxylate (10.2 g, 43.2 mmol) in anhydrous THF (50 mL) was added dropwise lithium borohydride (2.0 M solution in THF, 33.0 mL, 66.0 mmol) over 20 min. A solution of methanol (2.7 mL, 66.7 mmol) in anhydrous THF (10 mL) was then added over 30 min, and the mixture was stirred further overnight, allowing the temperature to rise to room temperature. The solvent was evaporated, and the residue was dissolved in ethyl acetate (70 mL), washed with hydrochloric acid (1 N, 30 mL×2), saturated sodium bicarbonate (30 mL), and brine (30 mL), dried with sodium sulfate, concentrated, and chromatographed (EtOAc/hexanes, 1:5 to 1:3) to give a colorless oil (6.3 g, 75%) as the corresponding alcohol. ¹H NMR (CDCl₃, 300 MHz) δ 3.02 (s, 1H), 4.73-4.74 (d, 2H, J=0.9 Hz), 7.17-7.18 (t, 1H, J=0.9 Hz). ¹³C NMR (CDCl₃, 75 MHz) δ 60.4, 118.6, 136.4, 156.7, IR (film, cm⁻¹) 1013, 1416, 2863, 2927, 3368.

To a solution of the alcohol (6.3 g, ˜32.5 mmol) and imidazole (2.4 g, 40.0 mmol) in anhydrous DMF (30 mL) was added a solution of tert-butyldimethylchlorosilane (6.0 g, 40.0 mmol) in anhydrous THF (20 mL) over 30 min. The mixture was stirred further overnight, diluted in water (30 mL), and extracted with ether (50 mL×4). The combined organics were washed with hydrochloric acid (1 N, 30 mL), saturated sodium bicarbonate (30 mL), and brine (30 mL), dried with sodium sulfate, concentrated, and chromatographed (EtOAc/hexanes, 1:10) to give a colorless oil (8.0 g, 60% for two steps). ¹H NMR (CDCl₃, 300 MHz) δ 0.11 (s, 6H), 0.94 (s, 9H), 4.81-4.82 (d, 2H, J=1.4 Hz), 7.13-7.14 (t, 1H, J=1.4 Hz). ¹³C NMR (CDCl₃, 75 MHz) δ −5.4, 18.3, 25.8, 62.0, 117.2, 135.5, 157.6. IR (film, cm⁻¹) 778, 838, 1014, 1105, 1138, 1257, 1424, 2857, 2929, 2954.

### EXAMPLE 8

### Synthesis of tert-Butyl (1S,3R) and (1R,3R)-1-(4-((tert-butyldimethylsilyloxy)methyl)thiazol-2-yl)-1-hydroxy-4-methylpentan-3-yl(methyl)carbamate (9a and 9b)

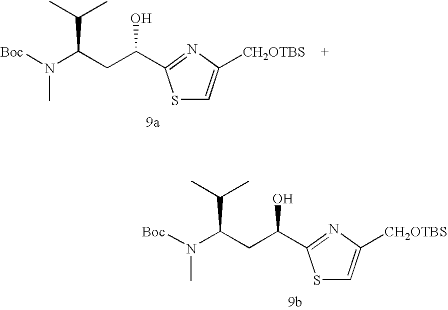

To an ice-cooled solution of 8 (0.62 g, 2.0 mmol) in anhydrous THF (10 mL) was added dropwise sec-butylmagnesium chloride (1.7 M, 1.2 mL, 2.0 mmol) in THF, and the mixture was stirred further for 30 min. A solution of 7 (0.23 g, 1.0 mmol) in anhydrous THF (5 mL) was then added dropwise over 10 min, and the mixture was stirred further overnight, during which time it gradually rose to room temperature. The reaction was then quenched by the addition of saturated ammonium chloride (10 mL), and the mixture was extracted with ethyl acetate (30 mL×2). The combined organics were washed with hydrochloric acid (1 N, 10 mL), saturated sodium bicarbonate (10 mL), and brine (10 mL), dried with sodium sulfate, evaporated, and chromatographed (Et₂O/hexanes, 1:3) to give two fractions of colorless oil.

The first fraction (0.18 g, 40%) was the desired (1R,3R)-isomer 9b; R_{f}=0.56 (Et₂O/hexanes, 1:3); [α]²³_{D}−12.0 (c 2.5, CH₂Cl₂). ¹H NMR (CDCl₃, 300 MHz) δ 0.10 (s, 6H), 0.89-0.96 (2d, 6H, J=6.5 Hz), 0.93 (s, 9H), 1.46-1.50 (2 s, 9H), 1.67-1.77 (m, 1H), 1.87-2.08 (m, 2H), 2.72 (s, 3H), 3.91-4.00 (dt, 1H, J=11.2, 5.7 Hz), 4.64-4.70 (dt, 1H, J=10.8, 3.1 Hz), 4.82-4.83 (d, 2H, J=1.1 Hz), 4.98-4.99 (d, 1H, J=3.4 Hz), 7.10-7.11 (t, 1H, J=1.2 Hz). ¹³C NMR (CDCl₃, 75 MHz) δ −5.4, 18.3, 20.1, 20.2, 25.9, 28.1, 28.3, 28.4, 29.7, 37.9, 57.7, 62.3, 69.1, 80.5, 113.1, 156.7, 158.4, 174.9. IR (film, cm⁻¹) 778, 839, 1102, 1136, 1256, 1366, 1472, 1662, 1693, 2858, 2930, 2959, 3400. HRMS (EI) calcd for C₂₂H₄₂N₂O₄SSi (M⁺) 458.2635. found 458.2638.

The second fraction (90 mg, 20%) was the (1R,3S)-isomer 9a; R_{f}=0.38 (Et₂O/hexanes, 1:3); [α]²³_{D}−620.1 (c 1.2, CH₂Cl₂). ¹H NMR (CDCl₃, 300 MHz) δ0.11 (s, 6H), 0.82-0.84 (d, 3H, J=6.5 Hz), 0.94 (s, 9H), 0.99-1.01 (d, 3H, J=6.5 Hz), 1.40-1.42 (2 s, 9H), 1.68-1.80 (m, 1H), 2.19-2.41 (m, 2H), 2.34-2.56 (2 s, 3H), 3.80-3.86 (dt, 1H, J=10.5, 3.3 Hz), 4.74-4.87 (m, 3H), 4.92-5.04 (m, 1H), 7.06-7.13 (2 s, 1H). ¹³C NMR (CDCl₃, 75 MHz) δ −5.4, −5.3, 18.4, 19.7, 20.3, 25.9, 28.5, 28.6, 30.2, 35.7, 58.0, 62.3, 70.7, 80.1, 113.1, 156.8, 157.6, 176.5. IR spectrum was identical to that of 9b. m/z (ESI) 459 ([M+H]⁺), 481 ([M+Na]⁺).

### EXAMPLE 9

### Synthesis of (1R,3R)-3-(tert-Butoxycarbonyl(methyl)amino)-1-(4-(hydroxymethyl)thiazol-2-yl)-4-methylpentyl acetate (10)

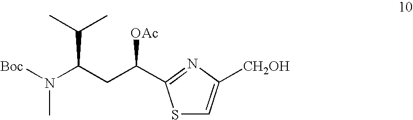

To an ice-cooled solution of 9b (85 mg, 0.18 mmol) and triethylamine (0.10 mL, 0.72 mmol) in dichloromethane (4 mL) was added acetyl chloride (0.05 mL, 0.70 mmol), and the mixture was stirred further for 3 hours, during which time it rose gradually to room temperature. The mixture was diluted in ether (40 mL), washed with saturated sodium bicarbonate (10 mL×2) and brine (10 mL), dried with sodium sulfate, and concentrated to give a yellow oil which was used without further purification. The oil was then dissolved in THF (1 mL) and treated with tetrabutylammonium fluoride (1.0 M solution in THF, 1.0 mL, 1.0 mmol) at room temperature overnight. The mixture was then diluted in ethyl acetate (40 mL), washed with brine (10 mL), dried with sodium sulfate, concentrated, and chromatographed (EtOAc/hexanes, 1:1) to give a colorless oil (45 mg, 63% for two steps). [α]²³_{D}+15.1 (c 1.5, CH₂Cl₂). ¹H NMR (CDCl₃, 300 MHz) δ 0.84-0.87 (d, 3H, J=6.6 Hz), 0.96-0.98 (d, 3H, J=6.6 Hz), 1.44 (s, 9H), 1.64-1.72 (m, 1H), 1.97-2.07 (m, 1H), 2.14 (s, 3H), 2.19-2.38 (m, 1H), 2.62-2.69 (2 s, 3H), 2.80-2.97 (2 bs, 1H), 4.03-4.14 (dt, 1H, J=11.1, 3.6 Hz), 4.74 (s, 2H), 5.80-5.85 (dd, 1H, J=11.6, 2.8 Hz, major rotomer), 5.89-5.93 (dd, 1H, J=9.0, 3.9 Hz, minor rotomer), 7.14-7.15 (2 s, 1H). ¹³C NMR (CDCl₃, 75 MHz) δ 19.6, 19.7, 19.9, 20.2, 20.9, 21.0, 28.0, 28.3, 28.4, 30.4, 30.7, 34.9, 56.3, 60.8, 60.9, 69.3, 70.5, 79.3, 70.7, 114.7, 115.0, 156.2, 156.3, 156.4, 169.5, 169.9, 170.2, 170.8. IR (film, cm⁻¹) 1157, 1223, 1367, 1689, 1755, 2971, 3434. HRMS (ESI) calcd for C₁₈H₃₀N₂O₅NaS ([M+Na]⁺) 409.1773. found 409.1780.

### EXAMPLE 10

### Synthesis of 2-((1R,3R)-1-Acetoxy-3-(tert-butoxycarbonyl(methyl)amino)-4-methylpentyl)thiazole-4-carboxylic acid (11)

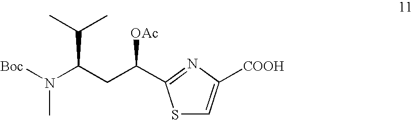

Dess-Martin periodinane (48 mg, 0.11 mmol) was added to a solution of 10 (30 mg, 0.08 mmol) in anhydrous dichloromethane (2 mL), and the mixture was stirred at room temperature for 6 hours. The mixture was diluted in ether (30 mL), washed with a mixture of sodium hydroxide (1.0 N, 5 mL) and sodium thiosulfate (1.0 M, 5 mL), saturated sodium bicarbonate (10 mL), and brine (10 mL), dried with sodium sulfate, and evaporated to give a colorless oil (29 mg, 99%) as the crude aldehyde. ¹H NMR (CDCl₃, 300 MHz) δ 0.85-0.87 (d, 3H, J=6.6 Hz), 0.96-0.99 (d, 3H, J=6.6 Hz), 1.43 (s, 9H), 1.64-1.76 (m, 1H), 2.07-2.13 (m, 1H), 2.16 (s, 3H), 2.31-2.41 (m, 1H), 2.70 (s, 3H), 4.04-4.14 (m, 1H), 5.84-5.89 (dd, 1H, J=11.6, 2.9 Hz, major rotomer), 5.92-5.96 (dd, 1H, J=9.8, 2.9 Hz, minor rotomer), (m, 1H), 8.13-8.14 (2 s, 1H), 9.99-10.00 (2 s, 1H).

The crude aldehyde (29 mg, 0.08 mmol) was then dissolved in tert-butyl alcohol (2 mL), and a solution of 2-methyl-2-butene in THF (2 M, 0.3 mL, 0.60 mmol) was added, followed by the dropwise addition of a mixture of sodium chlorite (39 mg, 0.43 mmol) and sodium dihydrogenphosphate monohydrate (0.13 g, 0.97 mmol) in water (1.0 mL). The mixture was stirred further at room temperature for 6 hours, and it was diluted in hydrochloric acid (0.1 N, 10 mL) and extracted with ethyl acetate (10 mL×3). The combined organics were dried with sodium sulfate, evaporated, and chromatographed (CH₂Cl₂/MeOH/AcOH, 95:5:0.5) to give a colorless oil (28 mg, 90% for two steps); [α]²³_{D}+5.0 (c 1.1, CH₂Cl₂). ¹H NMR (CDCl₃, 300 MHz) δ 0.85-0.87 (d, 3H, J=6.5 Hz), 0.96-0.98 (d, 3H, J=6.3 Hz), 1.44 (s, 9H), 1.66-1.71 (m, 1H), 2.17 (s, 3H), 2.23-2.32 (m, 1H), 2.64-2.70 (2 s, 3 H), 4.05-4.12 (m, 1H), 5.87-6.00 (m, 1H), 8.22 (s, 1H). ¹³C NMR (CDCl₃, 75 MHz) δ 15.2, 19.5, 19.7, 20.0, 20.3, 20.6, 20.8, 20.9, 28.4, 28.5, 29.7, 30.5, 34.8, 56.5, 65.8, 69.5, 70.7, 79.5, 80.0, 128.2, 146.9, 156.4, 156.5, 163.4, 169.4, 170.1, 170.6, 171.5, 175.9. IR (film, cm⁻¹) 1158, 1221, 1368, 1391, 1484, 1689, 1744, 2972, 3119. HRMS (ESI) calcd for C₁₈H₂₈N₂O₆NaS ([M+Na]⁺) 423.1566. found 423.1608.

### EXAMPLE 11

### Synthesis of di-tert-butyl (2R,4S)-5-hydroxy-4-methyl-1-phenylpentan-2-yliminodicarbonate (14)

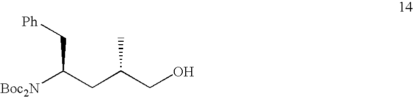

To a solution of 12 (1.0 g, 1.9 mmol) in anhydrous THF (15 mL) cooled in a dry ice/acetone bath was added butyllithium (1.6 M solution in hexanes, 1.8 mL, 2.5 mmol) dropwise over 5 minutes, and the mixture was stirred further for 30 min before a solution of di-tert-butyl dicarbonate (0.75 g, 3.3 mmol) in anhydrous THF (5 mL) was introduced in one portion. The mixture was stirred further overnight, during which time it gradually rose to room temperature. The reaction was quenched with saturated ammonium chloride (10 mL), and the mixture was extracted with ethyl acetate (30 mL×2). The combined organics were washed with hydrochloric acid (1 N, 10 mL) and brine (10 mL×2), dried with sodium sulfate, and evaporated to give a colorless oil which was used without further purification.

The oil obtained as above was dissolved in THF (2 mL) and treated with tetrabutylammonium fluoride (1.0 M solution in THF, 4.0 mL, 4.0 mmol) at room temperature overnight. The mixture was then diluted in ethyl acetate (70 mL), washed with brine (10 mL×2), dried with sodium sulfate, concentrated, and chromatographed (EtOAc/hexanes, 1:1) to give a colorless oil (0.44 g, 59% for two steps); [α]²³_{D}−69.2 (c 1.5, CH₂Cl₂). ¹H NMR (CDCl₃, 300 MHz) δ 0.94-0.97 (d, 3H, J=6.6 Hz), 1.40 (s, 18H), 1.61-1.88 (m, 3H), 2.78-2.85 (dd, 1H, J=13.4 Hz, 5.9 Hz), 3.14-3.22 (dd, 1H, J=13.4 Hz, 9.7 Hz), 3.44-3.56 (m, 2H), 4.46-4.55 (m, 1H), 7.15-7.28 (m, 5H). ¹³C NMR (CDCl₃, 75 MHz) δ 17.6, 27.9, 32.9, 36.3, 40.2, 57.5, 67.8, 81.9, 126.1, 128.2, 129.4, 139.0, 153.5. IR (film, cm⁻¹) 1145, 1346, 1699, 1738, 2932, 2978, 3436. m/z (ESI) 416 ([M+Na]⁺).

### EXAMPLE 12

### Synthesis of (2S,4R)-Allyl 4-(bis(tert-butoxycarbonyl)amino)-2-methyl-5-phenylpentanoate (15)

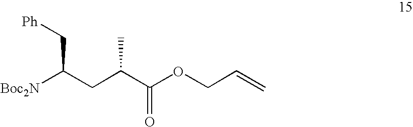

Alcohol 14 (0.39 g, 1.0 mmol) was oxidized by the same two-step sequence as described for 10 to give a colorless oil (0.68 g) as the corresponding crude carboxylic acid, which was dissolved in DMF (3 mL) and mixed with cesium carbonate (0.91 g, 2.8 mmol) and allyl bromide (1.0 mL, 11.5 mmol). After overnight stirring, the mixture was diluted in water (15 mL) and extracted with ethyl acetate (30 mL×2). The combined organics were washed with brine (15 mL×2), dried with sodium sulfate, evaporated, and chromatographed (EtOAc/hexanes, 1:7) to give a colorless oil (0.36 g, 81% for three steps); [α]²³_{D}−26.7 (c 1.0, CH₂Cl₂). ¹H NMR (CDCl₃, 300 MHz) δ 1.18-1.21 (d, 3H, J=7.1 Hz), 1.40 (s, 18H), 2.00-2.05 (m, 2 H), 2.50-2.57 (m, 1H), 2.80-2.86 (dd, 1H, J=13.5 Hz, 6.1 Hz), 3.12-3.20 (dd, 1H, J=13.4 Hz, 9.6 Hz), 4.45-4.65 (m, 3H), 5.18-5.32 (m, 2H), 5.85-5.94 (m, 1H), 7.17-7.27 (m, 5H). ¹³C NMR (CDCl₃, 75 MHz) δ 18.3, 27.9, 36.3, 36.8, 40.0, 57.3, 65.0, 81.8, 117.8, 126.2, 128.2, 129.4, 132.5, 138.7, 153.1, 175.7. IR (film, cm⁻¹) 1145, 1228, 1345, 1456, 1791, 1738, 2935, 2979. m/z (ESI) 470 ([M+Na]⁺).

### EXAMPLE 13

### Synthesis of (2S,4R)-Allyl 4-(2-((1R,3R)-1-acetoxy-3-(tert-butoxycarbonyl(methyl)amino)-4-methylpentyl)thiazole-4-carboxamido)-2-methyl-5-phenylpentanoate (16)

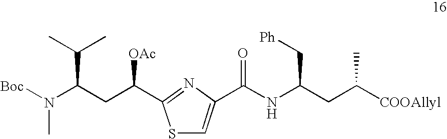

To a solution of 15 (0.20 g, 0.45 mmol) in dichloromethane (5 mL) was added trifluoroacetic acid (1 mL, 13 mmol), and the mixture was stirred at room temperature for 2 hours. The mixture was diluted in ethyl acetate (60 mL), washed with saturated sodium bicarbonate (15 mL×2), dried with sodium sulfate, and evaporated to give a colorless oil (0.13 g, 100%) as the crude amine. m/z (ESI) 248 ([M+H]⁺).

To a solution of 11 (82 mg, 0.21 mmol) and triethylamine (0.06 mL, 0.45 mmol) in anhydrous THF (4 mL) cooled to −20° C. was added dropwise isobutyl chloroformate (0.05 mL, 0.37 mmol), and the resulting white suspension was stirred further for 30 minutes. A solution of the above crude amine (0.13 g, 0.45 mmol) in anhydrous THF (2 mL) was then added via cannula, and the mixture was stirred further overnight, allowing the temperature to gradually rise to room temperature. The mixture was then diluted in ethyl acetate (70 mL), washed with brine (15 mL×2), dried with sodium sulfate, concentrated, and chromatographed (EtOAc/hexanes, 1:2) to give a white solid (0.10 g, 76% for two steps); mp 105.1-107.0° C.; [α]²³_{D}+9.9 (c 0.81, CH₂Cl₂). ¹H NMR (CDCl₃, 300 MHz) δ 0.92-0.94 (d, 3H, J=6.5 Hz), 1.01-1.06 (appar. t, 3H, J=7.1 Hz), 1.22-1.24 (d, 3H, J=7.0 Hz), 1.49 (s, 9H), 1.63-1.79 (m, 2H), 2.01-2.12 (m, 2H), 2.20-2.21 (2 s, 3H), 2.29-2.38 (m, 1H), 2.71-2.76 (m, 3 H), 2.89-3.04 (m, 2H), 4.10-4.16 (m, 1H), 4.47 (m, 1H), 4.58-4.60 (d, 2H, J=5.4 Hz), 5.21-5.34 (m, 2H), 5.83-5.87 (dd, 1H, J=11.7 Hz, 2.9 Hz), 5.89-5.99 (m, 1H), 7.15-7.18 (d, 1H, J=9.2 Hz), 7.25-7.31 (m, 5H), 8.05 (s, 1H). ¹³C NMR (CDCl₃, 75 MHz) δ 17.7, 17.8, 19.6, 19.7, 20.0, 20.3, 20.8, 20.9, 28.2, 28.4, 30.4, 30.6, 35.5, 36.6, 37.7, 37.8, 41.1, 41.2, 48.4, 48.5, 56.4, 65.1, 69.2, 70.8, 79.4, 79.7, 117.8, 123.1, 123.2, 126.5, 128.4, 129.4, 129.5, 132.3, 137.6, 137.7, 150.0, 150.2, 156.2, 160.3, 169.4, 170.1, 170.4, 175.7. IR (film, cm⁻¹) 1164, 1222, 1367, 1540, 1686, 1735, 2971, 3306, 3391. HRMS (EI) calcd for C₃₃H₄₇N₃O₇S (M⁺) 629.3135. found 629.3138.

### EXAMPLE 14

### Synthesis of Fmoc-Ile-F⁴

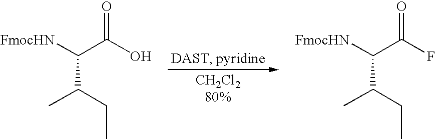

To a solution of Fmoc-Ile-OH (3.5 g, 10.0 mmol) and pyridine (0.81 mL, 10.0 mmol) in anhydrous dichloromethane (60 mL) was added via cannula (diethylamino)sulfur trifluoride (1.6 mL, 12.1 mmol) in dichloromethane (10 mL) over 10 min, and the mixture was stirred further at room temperature for 30 minutes. The mixture then was diluted in dichloromethane (40 mL), washed with ice-cold water (100 mL×2), dried with magnesium sulfate, filtered, concentrated, and recrystallized from dichloromethane/hexanes to give a white solid (2.8 g, 80%); mp 113.1-114.4° C. (lit. 115-116° C.; see Carpino, L. A. et al., J. Am. Chem. Soc. 1990, 112:9651); [α]²³_{D}+15.9 (c 0.51, EtOAc) (lit. [α]²³_{D}+15.6, c 0.51, EtOAc; see Carpino et al, Id.). ¹H NMR (CDCl₃, 300 MHz) δ 0.96-1.01 (t, 3H, J=7.4 Hz), 1.02-1.04 (d, 3H, J=6.9 Hz), 1.25-1.32 (m, 1H), 1.47-1.51 (m, 1H), 2.00 (m, 1H), 4.22-4.27 (appar. t, 1H, J=6.7 Hz), 4.47-4.50 (d, 2H, J=6.8 Hz), 4.53-4.57 (dd, 1H, J=8.7, 4.5 Hz), 5.15-5.18 (d, 1H, J=8.6 Hz), 7.31-7.36 (t, 2H, J=7.4 Hz), 7.40-7.45 (t, 2 H, J=7.4 Hz), 7.59-7.61 (d, 2H, J=7.4 Hz), 7.77-7.80 (d, 2H, J=7.7 Hz). ¹³C NMR (CDCl₃, 75 MHz) δ 11.4, 15.4, 25.0, 37.1, 47.1, 57.1-57.9 (d, J=57.2 Hz), 67.2, 120.0, 124.9, 127.1, 127.8, 141.3, 143.5, 155.9, 159.9-164.8 (d, J=372.1 Hz). ¹⁹F NMR (CDCl₃, 282 MHz) δ (CFCL3 as the external standard) 34.75, (film, cm⁻¹) 1082, 1256, 1451, 1520, 1705, 1843, 2968, 3324, m/z (ESI) 378 ([M+Na]⁺).

### EXAMPLE 15

### Synthesis of (2S,4R)-Allyl 4-(2-((5S,8R,10R)-5-sec-butyl-1-(9H-fluoren-9-yl)-8-isopropyl-7-methyl-3,6,12-trioxo-2,11-dioxa-4,7-diazatridecan-10-yl)thiazole-4-carboxami-do)-2-methyl-5-phenylpentanoate (17)

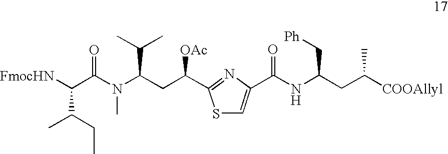

To a solution of 16 (53 mg, 84 μmol) in dichloromethane (2 mL) was added trifluoroacetic acid (0.3 mL, 3.9 mmol), and the mixture was stirred at room temperature for 10 hours. The mixture was diluted in ethyl acetate (60 mL), washed with saturated sodium bicarbonate (15 mL×2), dried with sodium sulfate, and evaporated to give colorless oil (56 mg, 100%) as the crude amine. m/z (ESI) 530 ([M+H]⁺), 552 ([M+Na]⁺).

The above free amine (56 mg, 84 μmol) was dissolved in anhydrous DMF (0.5 mL), and diisopropylethylamine (0.05 mL, 0.28 mmol) was added, followed by Fmoc-Ile-F (0.10 g, 0.28 mmol). The mixture was stirred further at room temperature for 18 h, and it was diluted in ethyl acetate (50 mL), washed with saturated sodium bicarbonate (10 mL) and brine (10 mL), dried with sodium sulfate, evaporated, and chromatographed (EtOAc/hexanes, 1:1) to give a colorless syrup (58 mg, 80%); [α]²³_{D}+0.29 (c 0.70, CH₂Cl₂). ¹H NMR (CDCl₃, 300 MHz) δ 0.82-0.84 (d, 3H, J=6.5 Hz), 0.91-0.96 (t, 3H, J=7.3 Hz), 0.99-1.01 (d, 3H, J=7.3 Hz), 1.03-1.05 (d, 3H, J=6.5 Hz), 1.19-1.21 (d, 3H, J=7.1 Hz), 1.61-1.77 (m, 3H), 2.00-2.11 (m, 3H), 2.19 (s, 3H), 2.31-2.38 (m, 1H), 2.65 (m, 1H), 2.86-2.96 (m, 2H), 3.00 (s, 3H), 4.20-4.24 (t, 1H, J=7.0 Hz), 4.38-4.44 (m, 3H), 4.54-4.58 (m, 3H), 5.18-5.31 (m, 2H), 5.43-5.46 (d, 1H, J=9.6 Hz), 5.63-5.66 (d, 1H, J=9.6 Hz), 5.82-5.94 (m, 1H), 7.10-7.13 (d, 1H, J=9.2 Hz), 7.22-7.34 (m, 7H), 7.38-7.43 (t, 2H, J=7.3 Hz), 7.58-7.60 (d, 2H, J=6.8 Hz), 7.76-7.78 (d, 2H, J=7.4 Hz), 8.02 (s, 1H). ¹³C NMR (CDCl₃, 75 MHz) δ 11.2, 16.0, 17.6, 19.5, 20.1, 20.8, 23.8, 29.6, 29.9, 34.6, 36.6, 37.3, 37.6, 41.0, 47.2, 48.3, 55.8, 65.1, 67.0, 69.5, 117.9, 119.9, 123.4, 125.0, 125.1, 126.5, 127.0, 127.6, 128.4, 129.6, 132.2, 137.5, 142.2, 141.3, 143.7, 143.9, 150.0, 157.3, 160.2, 169.9, 170.0, 173.6, 175.7. IR (film, cm⁻¹) 1222, 1464, 1495, 1645, 1717, 2966, 3291. m/z (ESI) 865 ([M+H]⁺), 887 ([M+Na]⁺).

### EXAMPLE 16

### Synthesis of N¹⁴-Desacetoxytubulysin H trifluoroacetic acid salt (1)

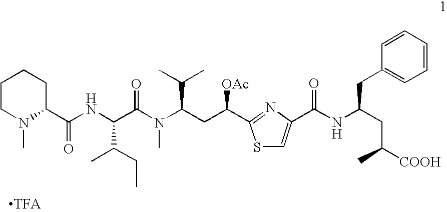

To a solution of 17 (17 mg, 20 μmol) in dichloromethane (0.5 mL) was added tris(2-aminoethyl)amine (0.05 mL, 0.33 mmol), and the mixture was stirred at room temperature for 3 hours. The mixture was then diluted in ethyl acetate (20 mL), washed with saturated sodium bicarbonate (5 mL) and brine (5 mL), dried with sodium sulfate, and evaporated to give a colorless oil (14 mg, 100%) as the crude amine. m/z (ESI) 643 ([M+H]⁺), 665 ([M+Na]⁺).

To a mixture of N-methyl-D-pipecolinic acid (9 mg, 63 μmol) (prepared from D-pipecolinic acid, Peltier, H. M. et al., J. Am. Chem. Soc., 2006, 128:16018) and N,N′-dicyclohexylcarbodiimide (18 mg, 86 μmol) in anhydrous DMF (0.4 mL) was added pentafluorophenol (12 mg, 65 μmol), and the mixture was stirred at room temperature overnight. The mixture was then filtered through a 0.2 μm Millex micro-filter unit to give a clear solution, which was used to dissolve the above crude amine. The mixture was stirred further at room temperature for 24 hours, and it was directly chromatographed (1:1 EtOAc/hexanes to wash out less polar impurities, followed by 2% MeOH in EtOAc to elute product) to give a yellow oil (15 mg) as the crude allyl ester of 1. m/z (ESI) 768 ([M+H]⁺), 790 ([M+Na]⁺).

The above crude allyl ester (15 mg, <17 μmol)) was then mixed with tetrakis(triphenylphosphine)palladium (0) (3 mg, 2.6 μmol) and dimedone (11 mg, 78 μmol) in THF (0.5 mL) under an Ar atmosphere. The mixture was stirred at room temperature overnight, and it was directly chromatographed (1:1 EtOAc/hexanes to wash out less polar impurities, followed by 10% MeOH in CH₂Cl₂ to elute product) to give a yellow oil. Further purification by semi-preparative HPLC (Dynamax Microsorb C18 column, 250 mm×10 mm; methanol/0.1% TFA in water system; methanol increased from 60% to 99% over 30 min; 2 mL/min) to give a colorless syrup (7.1 mg, 44%); τ_{R}=19.8 min; [α]²³_{D}−17.4 (c 0.46, MeOH). ¹H NMR (CD₃OD, 500 MHz) δ 0.84-0.86 (d, 3H, J=7.0 Hz), 0.93-0.96 (t, 3H, J=7.2 Hz), 1.01-1.02 (d, 3H, J=7.0 Hz), 1.03-1.04 (d, 3H, J=6.5 Hz), 1.17-1.18 (d, 3H, J=7.0 Hz), 1.20-1.23 (m, 1H), 1.56-1.70 (m, 3H), 1.74-1.81 (m, 2H), 1.89-1.95 (m, 4H), 1.98-2.04 (m, 1H), 2.15 (s, 3H), 2.16-2.18 (m, 1H), 2.28-2.42 (m, 2H), 2.53-2.58 (m, 1 H), 2.74 (s, 3H), 2.89-2.91 (d_{AB}, 2H, J=7.0 Hz), 3.04-3.11 (m, 1H), 3.12 (s, 3H), 3.44-3.49 (m, 1H), 3.73-3.77 (dd, 1H, J=13.5, 1.5 Hz), 4.36-4.42 (m, 2H), 4.70-4.74 (m, 1H), 5.70-5.73 (dd, 1H, J=11.0, 2.5 Hz), 7.17-7.19 (m, 1H), 7.23-7.24 (m, 4H), 8.07 (bs, 1H), 8.09 (s, 1H), 8.62-8.64 (d, 1H, J=8.0 Hz). ¹³C NMR (CD₃OD, 75 MHz) δ 11.3, 16.2, 18.5, 20.3, 20.5, 20.8, 22.2, 24.0, 25.2, 30.2, 30.9, 35.6, 37.4, 37.8, 39.1, 42.2, 42.9, 50.7, 56.0, 56.2, 68.0, 71.2, 125.2, 127.5, 129.4, 130.5, 139.5, 150.9, 162.8, 169.2, 171.9, 174.6, 180.0. IR (film, cm⁻¹) 1136, 1204, 1495, 1548, 1674, 2968. m/z (ESI) 728 ([M+H]⁺), 750 ([M+Na]⁺).

### EXAMPLE 17

### Methods for Assay of Antiproliferative Activity

General methods are known for determining the effect of compounds on proliferation of cells. See, e.g., Zhu et al., J. Med. Chem., 2006, 49, 2063-2076. Cells can be selected by one of skill in the art and can be maintained as exponentially growing cultures in a suitable medium (e.g., RPMI 1640 medium) with adjuvents, including FBS, penicillin, and glutamine, provided at levels known to those skilled in this field. For human cancer cells, e.g., human glioblastoma cell line T98G, the cells can be trypsinized and washed prior to formation of a cell suspension in appropriate buffer and plating of the cell suspension. Typical microtiter plate amounts can be 500-2000 cells/well. The cells can be allowed to attach and grow for a defined time period (e.g., 36 hours, 72 hours, 96 hours, or other defined period) before treatment with either vehicle (e.g., dimethylsulfoxide) or test agent (e.g., compound of the invention) followed by an additional defined time period (e.g., 36 hrs, 72 hrs, 96 hrs, and the like). At the end of the additional defined time period, viability of the cells can be determined by any of a variety of methods known in the art, including assays employing MTS (3-(4,5-dimethylthiazol-2-yl)-5-(3-carboxymethoxyphenyl)-2-(4-sulfophenyl)-2H-tetrazolium), as described by -Cory et al., Cancer Commun., 1991, 3, 207-212. The concentration of test agent required to elicit 50% inhibition of growth (i.e., GI₅₀) can be calculated and reported by standard techniques. Replicates are typically assayed to allow determination of appropriate statistical properties as known in the art.

### EXAMPLE 18

### Determination of GI₅₀ for Compounds of the Invention

Compounds of the invention with structures of Formulae Xa, XVb, and XVa were assayed for inhibition of growth of the T98G human glioblastoma cell line (Table II). As shown in Table 2, after 72 hrs exposure of cells as described above, the compounds having the structure of Formula Xₐ, XV_{b}, and XVₐ, demonstrated GI₅₀ values in the range 0.43 to 4.4 nM; compare paclitaxel (Wall, M. E. & Wani, M. C., Cancer Res. 1995, 55, 753-60) with GI₅₀ about 24 nM. At 96 hours exposure, the GI₅₀ decreased further to the range 0.35 to 16 nM.

TABLE II
GI₅₀ in T98G cell of selected tubulysin analogs

| Cmpd | 72 h | | 96 h | |
|---|---|---|---|---|
| Xₐ | 4.4 ± 11 | nM | 1.6 ± 0.8 | nM |
| XV_{b} | 0.59 ± 1.0 | μM | 0.40 ± 0.03 | μM |
| XVₐ | 0.43 ± 0.13 | μM | 0.35 ± 0.04 | μM |
| paclitaxel | 24 ± 9 | nM | 12 ± 7 | nM |

### EXAMPLE 19

### Preparation of Additional Analogs of Desacetoxytubulysin H

N¹⁴-Desacetoxytubulysin H was prepared via the synthetic approach summarized in Scheme 4. Protected amino alcohol 1 was N-acylated with Boc-anhydride and desilylated with tetrabutylammonium fluoride (TBAF) to give 2. Oxidation of the alcohol to the acid, followed by allylation, provided ester 3 in 81% yield. The N-terminus was deprotected and coupled with segment 4 to give thiazole 5. After two additional coupling reactions with Fmoc-L-leucine and N-methylpipecolate (Mep), the desired N¹⁴-desacetoxytubulysin H was obtained after Pd(0)-catalyzed deallylation of the C-terminal ester group. Intermediate 6 also was used in the coupling with N-methylsarcosine to give an analog designated “WZY-III-69A” (Scheme 5). In a longer synthetic sequence, the C(11)-epimer of WZY-III-69A, designated “WZY-III-64A,” was obtained from the acid derived from thiazole 8 and amine 9 via intermediate tripeptide and tetrapeptide 10 and 11, respectively.

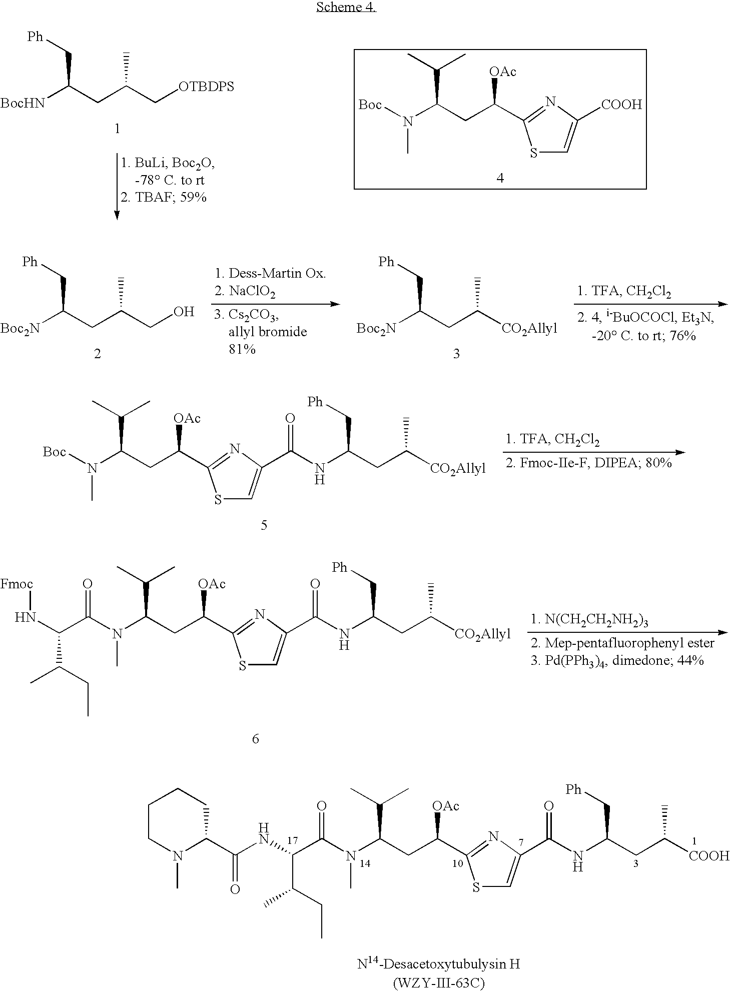

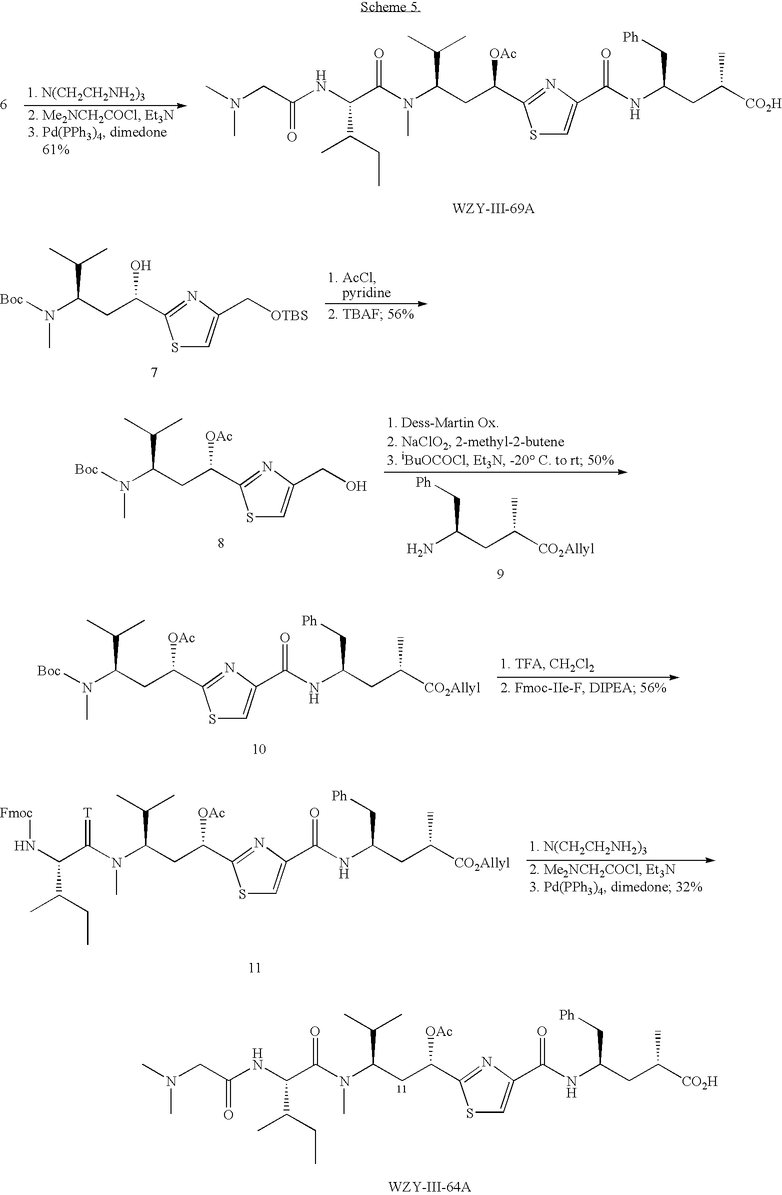

(2S,4R)-4-(2-((6S,9R,11R)-6-sec-Butyl-9-iso-propyl-2,8-dimethyl-4,7,13-trioxo-12-oxa-2,5,8-triazatetradecan-11-yl)thiazole-4-carboxamido)-2-methyl-5-phenylpentanoic acid (WZY-III-69A). Tris(2-aminoethyl)amine (51 mg, 0.35 mmol) was added to a solution of 6 (23 mg, 27 μmol) in dichloromethane (1 mL). The reaction mixture was stirred at room temperature for 1 hour, diluted with ethyl acetate (20 mL), washed with saturated sodium bicarbonate (5 mL) and brine (5 mL), dried (Na₂SO₄), and concentrated under vacuum to give the crude amine as a colorless oil.

Dimethylaminoacetyl chloride hydrochloride (85%, 27 mg, 0.14 mmol) was added to a mixture of this oil and triethylamine (0.04 mL, 0.29 mmol) in anhydrous DMF (0.6 mL), and the reaction mixture was stirred at room temperature for 24 hours. The mixture was diluted in ethyl acetate (40 mL), washed with brine (10 mL), dried (Na₂SO₄), and concentrated to give the crude allyl ester (23 mg) as a yellow oil: MS (ESI) m/z 750 ([M+Na]⁺), 728 ([M+H]⁺).

A solution of this allyl ester (23 mg), tetrakis(triphenylphosphine)-palladium(0) (5 mg, 4.3 [symbol] mol) and dimedone (10 mg, 71 [symbol] mol) in THF (0.6 mL) under an Ar atmosphere was stirred at room temperature for 1 day. After evaporation of the volatiles, the residue was purified by chromatography on SiO₂ (EtOAc/hexanes, 1:1, to wash out less polar impurities, followed by 10% MeOH in CH₂Cl₂ to elute the product) to give a yellow oil. Further purification by semi-preparative HPLC (Dynamax Microsorb C-18 column, 250 mm×10 mm; methanol/0.1% TFA in a water/methanol gradient from 70% to 99% over 20 minutes, then kept at 99% for 20 minutes; 5 mL/min) gave the trifluoroacetic acid salt of WZY-III-69A (13 mg, 61% for three steps) as a white foam: τ_{R}=19.0 min; [α]_{D}²³−25.1 (c 0.53, MeOH); ¹H NMR (CD₃OD, 500 MHz) 8.08 (s, 1H), 7.24-7.23 (m, 4H), 7.19-7.15 (m, 1H), 5.70 (dd, 1H, J=10.5, 3.0 Hz), 4.77 (d, 1H, J=7.0 Hz), 4.42-4.36 (m, 2H), 4.02, 3.95 (d_{AB}, 2H, J=15.8 Hz), 3.11 (s, 3H), 2.92-2.89 (m, 2H), 2.91 (bs, 6H), 2.57-2.53 (m, 1H), 2.42-2.22 (m, 3H), 2.15 (s, 3H), 2.04-1.98 (m, 1H), 1.94-1.86 (m, 2H), 1.71-1.60 (m, 2H), 1.17 (d, 3H, J=7.0 Hz), 1.04 (d, 3H, J=6.5 Hz), 1.01 (d, 3H, J=6.5 Hz), 0.94 (t, 3H, J=6.5 Hz), 0.85 (d, 3H, J=7.0 Hz); ¹³C NMR (CD₃OD, 75 MHz) δ 179.9, 174.5, 171.8, 171.6, 165.4, 162.8, 150.8, 139.5, 130.5, 129.3, 127.4, 125.1, 71.3, 59.1, 56.0, 50.6, 44.4, 42.2, 39.1, 37.8, 37.5, 35.6, 31.0, 25.2, 20.8, 20.5, 20.3, 18.5, 16.3, 11.4; IR (film) 2968, 2938, 2878, 1674, 1652, 1548, 1203, 1135 cm⁻¹; HRMS (ESI) calcd for C₃₅H₅₄N₅O₇S ([M+H]⁺) 688.3744. found 688.3769.

(1S,3R)-3-(tert-Butoxycarbonyl(methyl)amino)-1-(4-(hydroxymethyl)thiazol-2-yl)-4-methylpentyl acetate (8). Acetyl chloride (0.05 mL, 0.70 mmol) was added to an ice-cooled solution of 7 (0.117 g, 0.26 mmol) and pyridine (0.10 mL, 1.2 mmol) in dichloromethane (4 mL), and the mixture was stirred further for 3 hours, during which time the temperature gradually rose to room temperature. The mixture was diluted in ether (40 mL), washed with saturated sodium bicarbonate (10 mL×2) and brine (10 mL), dried (Na₂SO₄), and concentrated to give a yellow oil which was used without further purification. The oil was dissolved in THF (1 mL) and treated with tetrabutylammonium fluoride (1.0 M solution in THF, 1.0 mL, 1.0 mmol) at room temperature overnight. The mixture was diluted in ethyl acetate (40 mL), washed with brine (10 mL), dried (Na₂SO₄), concentrated, and purified by chromatography on SiO₂ (EtOAc/hexanes, 1:1) to give 8 (55 mg, 56% for two steps) as a colorless oil: [α]_{D}²³−52.6 (c 0.62, CH₂Cl₂). ¹H NMR analysis at room temperature showed a 1:1 mixture of rotamers. Major rotamer: ¹H NMR (CDCl₃, 300 MHz) δ 7.20 (s, 1H), 5.99-5.90 (m, 1H), 4.77 (s, 2 H), 3.77-3.68 (m, 1H), 2.71 (s, 3H), 2.46-2.39 (m, 1H), 2.12 (s, 3H), 1.73-1.63 (m, 1H), 1.44 (s, 9H), 0.95 (d, 3H, J=6.6 Hz), 0.81 (d, 3H, J=6.7 Hz); ¹³C NMR (CDCl₃, 75 MHz) δ 169.7, 169.0, 156.6, 156.1, 115.1, 79.6, 71.4, 61.1, 57.5, 35.1, 31.0, 28.4, 20.9, 19.9, 19.2; Characteristic peaks of the minor rotamer: ¹H NMR (CDCl₃, 300 MHz) δ 7.16 (s, 1H), 4.72, 4.70 (d_{AB}, 2H, J=12.9 Hz), 2.70 (s, 3H), 2.35-2.31 (m, 1H), 2.10 (s, 3H), 1.35 (s, 9H), 0.91 (d, 3H, J=6.6 Hz), 0.78 (d, 3H, 6.7 Hz); ¹³C NMR (CDCl₃, 75 MHz) δ 168.4, 156.6, 115.2, 79.3, 61.0, 31.2, 28.5, 21.0, 20.3, 19.5. IR (film) 3435, 2970, 2931, 2874, 1744, 1689, 1367, 1229, 1149 cm⁻¹; HRMS (ESI) calcd for C₁₈H₃₀N₂O₅NaS ([M+Na]⁺) 409.1773. found 409.1780.

(2S,4R)-Allyl 4-(2-((1S,3R)-1-acetoxy-3-(tert-butoxycarbonyl(methyl)amino)-4-methylpentyl)thiazole-4-carboxamido)-2-methyl-5-phenylpentanoate (10). Dess-Martin periodinane (71 mg, 0.17 mmol) was added to a solution of 8 (44 mg, 0.11 mmol) in anhydrous dichloromethane (2 mL). The reaction mixture was stirred at room temperature for 6 hours, diluted with ether (30 mL), washed with a mixture of sodium hydroxide (1.0 N, 5 mL) and sodium thiosulfate (1.0 M, 5 mL), saturated sodium bicarbonate (10 mL), and brine (10 mL), dried (Na₂SO₄), and concentrated under vacuum to give the crude aldehyde which was used without further purification.

A solution of this crude aldehyde in tert-butyl alcohol (2 mL) was treated with a solution of 2-methyl-2-butene in THF (2 M, 0.3 mL, 0.60 mmol), followed by the drop-wise addition of a mixture of sodium chlorite (62 mg, 0.55 mmol) and sodium dihydrogenphosphate monohydrate (0.14 g, 1.0 mmol) in water (2.0 mL). The reaction mixture was stirred further at room temperature for 6 hours, diluted with hydrochloric acid (0.1 N, 10 mL), and extracted with ethyl acetate (10 mL×3). The combined organic layers were dried (Na₂SO₄) and concentrated under vacuum to give the crude acid as a colorless oil.

To a solution of this oil and triethylamine (0.04 mL, 0.30 mmol) in anhydrous THF (2 mL) cooled to −20° C. was added drop-wise iso-butyl chloroformate (0.03 mL, 0.22 mmol), and the resulting white suspension was stirred further for 30 minutes. A solution of 9 (59 mg, 0.24 mmol) in anhydrous THF (1 mL) then was added via cannula, and the mixture was stirred overnight, allowing the temperature to rise gradually to room temperature. The mixture then was diluted with ethyl acetate (70 mL), washed with brine (15 mL×2), dried (Na₂SO₄), concentrated, and purified by chromatography on SiO₂ (EtOAc/hexanes, 1:2) to give 10 (36 mg, 50% for three steps) as a colorless oil: [α]_{D}²³−6.0 (c 0.53, CH₂Cl₂). ¹H NMR analysis at room temperature showed a 4:3 mixture of rotamers. Major rotamer: ¹H NMR (CDCl₃, 300 MHz) δ 8.01 (s, 1H), 7.34-7.19 (m, 5H), 7.11 (d, 1H, J=9.0 Hz), 5.96-5.79 (m, 2H), 5.30-5.14 (m, 2H), 4.53 (dd, 2H, J=10.5, 5.7 Hz), 4.47-4.38 (m, 1H), 4.13-4.00 (m, 1H), 3.03 (dd, 1H, J=13.5, 5.6 Hz), 2.91 (d, 1H, J=6.3 Hz), 2.78 (dd, 1 H, J=13.6, 7.6 Hz), 2.74 (s, 3H), 2.67-2.47 (m, 2H), 2.27-1.92 (m, 2H), 2.17 (s, 3 H), 1.76-1.53 (m, 2H), 1.46 (s, 9H), 1.19 (d, 3H, J=7.5 Hz), 0.89 (d, 3H, J=6.5 Hz), 0.82 (d, 3H, J=6.6 Hz); ¹³C NMR (CDCl₃, 75 MHz) δ 175.7, 169.6, 168.2, 160.7, 156.0, 150.4, 138.1, 132.3, 129.4, 128.3, 126.3, 124.2, 122.7, 117.8, 79.1, 71.1, 65.0, 65.1, 57.0, 49.1, 41.8, 37.3, 36.7, 35.0, 30.9, 28.4, 21.0, 20.1, 19.3, 18.0. Characteristic signals of the minor rotamer: ¹H NMR (CDCl₃, 300 MHz) δ 8.09 (s, 1 H), 7.91 (d, 1H, J=8.7 Hz), 3.03 (dd, 1H, J=13.5, 5.6 Hz), 2.72 (s, 3H), 2.09 (s, 3 H), 1.17 (d, 3H, J=7.4 Hz), 0.97 (d, 3H, J=6.5 Hz); ¹³C NMR (CDCl₃, 75 MHz) δ 175.6, 169.7, 160.3, 155.9, 149.9, 137.5, 132.2, 129.4, 128.4, 126.5, 118.0, 79.6, 70.3, 65.0, 48.5, 41.1, 37.9, 36.6, 28.2, 20.9, 20.3, 19.5, 17.7; IR (film) 3304, 2970, 2934, 2875, 1736, 1680, 1542, 1367, 1227, 1148 cm⁻¹; HRMS (EI) calcd for C₃₃H₄₇N₃O₇S 629.3135. found 629.3157.

(2S,4R)-Allyl 4-(2-((5S,8R,10S)-5-sec-butyl-1-(9H-fluoren-9-yl)-8-isopropyl-7-methyl-3,6,12-trioxo-2,11-dioxa-4,7-diazamidecan-10-yl)thiazole-4-carboxamido)-2-methyl-5-phenylpentanoate (11). To a solution of 10 (26 mg, 41 [symbol] mol) in dichloromethane (1 mL) was added trifluoroacetic acid (0.2 mL, 2.6 mmol). The reaction mixture was stirred at room temperature for 7 hours, diluted with ethyl acetate (60 mL), washed with saturated sodium bicarbonate (15 mL×2), dried (Na₂SO₄), and concentrated under vacuum to give the crude amine (24 mg) as a yellow oil: MS (ESI) m/z 552 ([M+Na]⁺), 530 ([M+H]⁺).

A solution of this amine in anhydrous CH₂Cl₂ (0.6 mL) was treated with diisopropylethylamine (0.03 mL, 0.17 mmol) and Fmoc-Ile-F (32 mg, 90 [symbol] mol), stirred at room temperature for 10 hours, diluted with ethyl acetate (50 mL), washed with saturated sodium bicarbonate (10 mL) and brine (10 mL), dried (Na₂SO₄), concentrated under vacuum, and purified by chromatography on SiO₂ (EtOAc/hexanes, 1:1) to give 11 (20 mg, 56% for two steps) as a white foam: [α]_{D}²³−6.5 (c 0.34, CH₂Cl₂); ¹H NMR analysis at room temperature showed a 4.8:1 mixture of rotamers. Major rotamer: ¹H NMR (CDCl₃, 300 MHz) δ 7.98 (s, 1H), 7.78 (d, 2 H, J=7.6 Hz), 7.65 (d, 2H, J=7.5 Hz), 7.42 (t, 2H, J=7.3 Hz), 7.32 (t, 2H, J=7.3 Hz), 7.27-7.22 (m, 5H), 7.04 (d, 1H, J=9.6 Hz), 5.96-5.84 (m, 2H), 5.70 (d, 1H, J=9.9 Hz), 5.28 (dd, 1H, J=17.2, 1.5 Hz), 5.20 (d, 1H, J=10.4 Hz), 4.57-4.25 (m, 7 H), 3.05-2.85 (m, 2H), 2.74 (s, 3H), 2.67-2.52 (m, 2H), 2.35-2.03 (m, 4H), 1.98 (s, 3H), 1.75-1.69 (m, 3H), 1.19 (d, 6H, J=7.1 Hz), 0.99 (d, 3H, J=6.7 Hz), 0.91 (d, 3H, J=6.2 Hz), 0.79 (d, 3H, J=6.5 Hz); ¹³C NMR (CDCl₃, 75 MHz) δ 175.8, 171.5, 170.8, 167.7, 160.5, 156.4, 150.5, 143.8, 141.3, 138.0, 132.4, 129.5, 128.3, 127.8, 127.1, 126.3, 125.0, 122.9, 120.0, 117.9, 71.9, 67.1, 65.0, 58.7, 54.7, 49.1, 47.2, 41.4, 37.9, 37.7, 36.8, 34.6, 30.6, 30.2, 24.9, 22.4, 20.0, 19.5, 18.0, 15.6, 11.5; Characteristic signals of the minor rotamer: ¹H NMR (CDCl₃, 300 MHz) δ 8.12 (s, 1 H), 2.77 (s, 3H); ¹³C NMR (CDCl₃, 75 MHz) δ 137.5, 129.3, 31.3; IR (film) 3273, 2965, 2876, 1729, 1643, 1541, 1452, 1410, 1258 cm⁻¹; HRMS (ESI) calcd for C₄₉H₆₁N₄O₈S ([M+H]⁺) 865.4210. found 865.4154.

(2S,4R)-4-(2-((6S,9R,11S)-6-sec-Butyl-9-isopropyl-2,8-dimethyl-4,7,13-trioxo-12-oxa-2,5,8-triazatetradecan-11-yl)thiazole-4-carboxamido)-2-methyl-5-phenylpentanoic acid (WZY-III-64A). According to the protocol used for WZY-III-69A, supra, 11 (7.5 mg, 8.7 [symbol] mol) provided the trifluoroacetic acid salt of WZY-III-64A (2.2 mg, 32% for three steps) as a yellow oil: [α]_{D}²³−36.4 (c 0.22, MeOH); ¹H NMR analysis at room temperature showed a 2.8:1 mixture of rotamers. Major rotamer: ¹H NMR (CD₃OD, 300 MHz) δ 8.11 (s, 1H), 7.23-7.13 (m, 5H), 6.00 (dd, 1H, J=8.6, 3.8 Hz), 4.57 (d, 1H, J=5.1 Hz), 4.42-4.31 (m, 2H), 4.07 (s, 2H), 2.94 (s, 6H), 2.92-2.90 (m, 2H), 2.85 (s, 3H), 2.57-2.16 (m, 4H), 2.03 (s, 3H), 1.90-1.64 (m, 3H), 1.38-1.24 (m, 2H), 1.16 (d, 3H, J=6.9 Hz), 0.95 (d, 3H, J=6.0 Hz), 0.91-0.80 (m, 6H), 0.79 (d, 3H, J=6.3 Hz); Characteristic ¹³C NMR peaks (CDCl₃, 75 MHz)

δ 180.0, 174.6, 172.9, 171.4, 166.4, 139.5, 130.4, 129.3, 127.4, 73.2, 59.1, 50.7, 44.4, 39.0, 37.9, 31.4, 26.1, 22.3, 20.5, 19.8, 18.5, 16.0, 11.8. Characteristic signals of the minor rotamer: ¹H NMR (CDCl₃, 300 MHz) δ 8.19 (s, 1H), 6.15-6.05 (m, 1H), 4.51 (d, 1H, J=5.4 Hz), 4.03 (s, 2H), 2.73 (s, 3H), 1.86 (s, 3H), 1.04 (d, 3H, J=6.6 Hz); MS (ESI) m/z 710 ([M+Na]⁺), 688 ([M+H]⁺).

### Automated High Content Cellular Analyses

The effects of tubulysin analogs on mitotic arrest and cellular microtubules were studied. HeLa human cervical carcinoma cells (10,000 per well) were plated in collagen-1 coated 384-well microplates and were treated with vehicle (DMSO) or ten two-fold concentration gradients of test agents within 4-6 hours of seeding. Cells were incubated for 20 hours at 37° C., fixed with formaldehyde, and labeled with 10 μg/mL Hoechst 33342 in Hank's balanced salt solution (HBSS). Cells were permeabilized with 0.5% (w/w) Triton X-100 for 5 min at room temperature and incubated with a primary antibody cocktail consisting of an HBSS solution containing rabbit polyclonal anti-phosphohistone H3 (Ser10, 1:500, Upstate), and mouse monoclonal anti-α-tubulin (1:3000, Sigma), followed by a mixture of FITC-labeled donkey anti-mouse IgG (1:500) and Cy3-labeled donkey anti-rabbit IgG (1:500). Cells were rinsed once with HBSS and stored at 4° C. in HBSS until analysis.

Microplates were analyzed with an ArrayScanII instrument (Cellomics, Pittsburgh, Pa.) using the Target Activation Bioapplication (Cellomics, Inc.). Within the application, 1,000 individual cells in each well were imaged at three different wavelengths, using an Omega XF93 filter set at excitation/emission wavelengths of 350/461 nm (Hoechst), 494/519 nm (FITC), and 556/573 nm (Cy3). The following parameters were used for data analysis: average nuclear intensity, nuclei per field, average nuclear FITC intensity, and average nuclear Cy3 intensity. A nuclear mask was generated from Hoechst 33342-stained nuclei. Microtubule density and histone H3 phosphorylation were measured in the FITC and Cy3 channel, respectively. Microtubule density was defined as the average green (FITC) pixel intensity in an area defined by the nuclear mask. For determination of mitotic index and nuclear condensation, thresholds for Hoechst 33342 and phosphohistone-H3 intensities were defined as the average Hoechst 33342 or Cy3 intensity plus one standard deviation from twenty-eight vehicle-treated wells placed in the center of the microplate.

Cells were classified as positive if their average Hoechst 33342 or Cy3 intensity exceeded this threshold. To illustrate visually the effects of test agents on cellular microtubules and mitotic arrest, the identical 384-well plates then were used to acquire higher resolution images of selected wells.

### Antiproliferative Assay

Human glioblastoma T98G cells (American Type Culture Collection, Manassas, Va.) were maintained as exponentially growing cultures in an RPMI 1640 medium with 10% FBS, 1% penicillin and 1% glutamine. The cells were seeded into 96-well plates and allowed to attach and grow for 48 hours. One plate of cells was used for a time zero cell number determination (N=16), and cells in other plates were treated for 72 hours or 96 hours with either DMSO (1% v/v; N=8 for each plate) or a range of concentrations, in quadruplicate, of test agents. Cell number was determined spectrophotometrically at 490 nm minus absorbance at 630 nm after exposure to 3-(4,5-dimethylthiazol-2-yl)-5-(3-carboxymethoxyphenyl)-2-(4-sulfophenyl)-2H-tetrazolium (MTS) and N-methylphenazine methylsulfate. The fifty percent growth inhibitory concentrations (GI₅₀) of test agents were calculated from the spectrophotometrically determined growth of the control cells over the 72- or 96-hour period.

### Inhibition of Tubulin Assembly

Bovine brain tubulin was isolated to electrophoretic homogeneity from fresh brains via methodology described by Hamel and Lin, Biochemistry 23: 4173-84 (1984). Tubulin (final concentration 10 μM; 1 mg/mL) was preincubated with test agents dissolved in DMSO (4% v/v final concentration) and monosodium glutamate (0.8 M final concentration, pH 6.6) for 15 minutes at 30° C. The reaction mixture was cooled to 0° C., and GTP (0.4 mM final concentration) was added. Reaction mixtures were transferred to cuvettes held at 2.5° C. in a Beckmann DU 7400 multichannel, temperature-controlled (Peltier unit) spectrophotometer, reading absorbance (turbidity) at 350 nm in each cuvette every 15 seconds. Baselines were established and temperature was quickly raised to 30° C. (in approximately 1 minute). After 20 minutes, the temperature was returned to 2.5° C. The turbidity value after 20 minutes at 30° C. for GTP alone was assigned as 100% assembly, and for DMSO alone as 0% assembly. The IC₅₀ was calculated by linear regression of the percent assembly values at the same time point obtained with 0.625, 1.25, 2.5, 5, 10 and 20 μM test agent. In experiments to test for disassembly of preformed tubulin polymer, the test agent was added after 7 minutes of GTP-induced assembly.

### Inhibition of the Binding of Radiolabeled Tubulin Ligands

The new compounds were tested for their abilities to bind at the vinca domain and at the related peptide/depsipeptide site on tubulin using a modification of a centrifugal gel filtration method described by Bai et al., Mol. Pharmacol. 7: 965-76 (1995). In the initial screens, each 0.32 mL reaction mixture contained 5 μM test agent and 10 μM [³H]vinblastine (specific activity 8.50 Ci/mmol; GE Healthcare Bio-Sciences, Picataway, N.J.) or 10 μM [³H]dolastatin 10 (specific activity 1.96 Ci/mmol; obtained from the Drug Synthesis and Chemistry Branch of the National Cancer Institute) predissolved in DMSO (final concentration 1% v/v) and 10 μM bovine brain tubulin, all in 0.1 M 2-(4-morpholino)ethanesulfonic acid (Mes), pH 6.9, containing 0.5 mM MgCl₂. The mixture was incubated for 30 min at room temperature. Two 0.15 mL aliquots of the reaction mixture were applied to duplicate 2 mL beds of pre-swollen (in Mes, pH 6.9, containing 0.5 mM MgCl₂) Sephadex G-50 (superfine) contained in Handee columns (Pierce Biotechnology, Rockford, Ill.). The columns were centrifuged at 800×g for 4 minutes and the protein concentration in the effluent was determined, using the bicinchoninic acid assay. The amount of radiolabeled ligand bound to tubulin was determined using scintillation spectrometry. Each value represents the mean ±SD of four determinations. The average stoichiometry of binding for [³H]vinblastine and [³H]dolastatin 10 in the control reaction mixture was in each case 0.63 mol of radiotracer per mole of tubulin. Inhibition was calculated as the percent difference relative to that of the control incubation with DMSO (i.e., without test agent—assigned as 0% inhibition). For IC₅₀ determinations, test agents (including normoisotopic dolastatin 10, also from the Drug Synthesis Branch of the NCI) were incubated over a range of concentrations (0.5-100 μM) with 10 μM [³H]vinblastine or [³H]dolastatin 10 and bovine brain tubulin, as outlined above (each concentration determined in quadruplicate). GraphPad Prism was used to fit the resulting data to the Hill equation to determine the IC₅₀ values.

### High-Content Analysis of Mitotic Arrest

A hallmark of microtubule perturbing agents is a blockage of the cell cycle in mitosis. Since tubulysin A has been reported to destabilize microtubules (Khalil et al., ChemBioChem. 7: 678-83 (2006), a multiparameter, high-content analysis was conducted to illuminate the effects of the above-described analogs on cellular microtubule perturbation, apoptotic morphology, cell cycle arrest, and histone H3 phosphorylation as molecular marker of mitosis.

Asynchronously growing HeLa cells were treated for 20 hours with each compound in collagen-coated 384 well microplates, fixed, and incubated with primary antibodies for tubulin and the mitotic marker protein phosphohistone H3, followed by FITC and Cy3-conjugated secondary antibodies, respectively. Cells were detected by nuclear counterstaining with Hoechst 33342, which also provided information about chromatin condensation and cell density as markers of cell death.

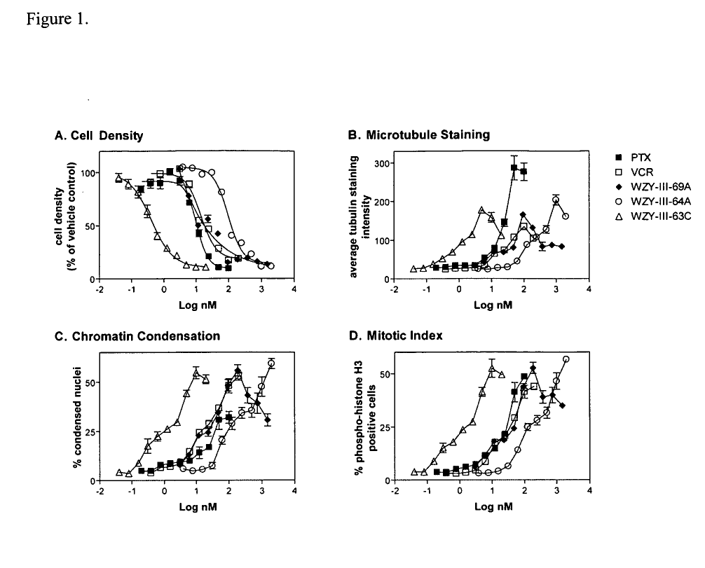

Vehicle treated cells had highly organized microtubules and a low percentage of mitotic cells. Mid-nanomolar concentrations of paclitaxel caused formation of bright microtubule bundles, whereas cells treated with vincristine had generally disorganized microtubules and perhaps a lower overall tubulin content, in keeping with results reported by Wipf et al., J. Am. Chem. Soc. 122: 9391-95 (2000). N¹⁴-Desacetoxytubulysin H (designated “WZY-III-63C,” FIG. 1C) showed microtubule disorganization similar to that seen with vincristine, as well as an increase in the percentage of mitotic cells, but this agent appeared to be almost two orders of magnitude more potent than vincristine or paclitaxel. Hoechst 33342 staining revealed the presence of condensed and fragmented nuclei characteristic of apoptosis. In other studies there was evidence for caspase 3 activation as a biochemical indicator of apoptosis (data not shown).

FIG. 1 shows the quantitative assessment of mitotic arrest and nuclear morphology by N¹⁴-desacetoxytubulysin H (WZY-III-63C) compared with the analogs WZY-III-69A and WZY-III-64A, paclitaxel, and vincristine. All agents caused cell loss, either through detachment or lysis (FIG. 1A), condensed chromatin (FIG. 1C), and an increase in the percentage of cells with elevated phosphohistone H3 levels (FIG. 1D). The effect of agents on measurements of cellular microtubules varied depending on their proposed mechanism of action. All agents increased microtubule density (FIG. 1B) with vincristine and the tubulysins showing a biphasic response. At lower concentrations, microtubule density increased, presumably due to cell rounding and concentration of microtubules in a smaller area. At higher concentrations, microtubule density measurements decreased due the drug-induced shift of the dynamics of microtubules toward tubulin monomers and preferential extraction of monomeric tubulin during the permeabilization process. This type of behavior has previously been observed for microtubule destabilizing agents (Wipf et al., Chem. Boil. Drug Des. 67: 66-73 (2006)). In contrast, microtubule density steadily increased in cells treated with paclitaxel, which causes formation of stable microtubules and microtubule bundles. These data indicate that N¹⁴-desacetoxytubulysin H (WZY-III-63C) may have a mechanism of action distinct from paclitaxel and more closely related to that of the vinca alkaloids.

Table III summarizes the data from the high-content analysis for all of the agents tested. While EC₅₀ values were readily obtained from the cell density curves, the shape and form of the mitotic index, chromatin condensation, and microtubule density graphs precluded IC₅₀ determinations. Accordingly, the minimum detectable effective concentration (MDEC) was determined, as an alternative measurement of drug activity. See Wipf et al. (2000), supra. Measurements from all parameters were well correlated, and there was a clear SAR among the three tubulysin analogs. WZY-III-69A was as active as paclitaxel or vincristine. The analog WZY-III-64A, which differs from WZY-III-69A solely by the configuration at the acetate-bearing stereocenter at C¹¹, was about five-fold less active. In contrast, N¹⁴-desacetoxytubulysin H (WZY-III-63C) had activity in the picomolar range and was almost 50-fold more potent than paclitaxel, vincristine, or WZY-III-69A in all of the endpoints measured. WZY-III-63C has the natural configuration at C¹¹ and shares the Mep N-terminal amino acid residue common to all tubulysins, whereas analogs WZY-III-64A and WZY-III-69A have simplified N-methylsarcosine residues at this position.

### Biochemical Evaluation

In another cancer cell line (human T98G glioblastoma cells) where the antiproliferative assay was performed by a traditional cell counting method, i.e., an MTS assay, results similar to those with HeLa cells were found (Table IV). The cell-based assays showed the test compounds to be antiproliferative as well as antimitotic, and strongly suggested tubulin and microtubules to be a primary target. Therefore, the samples were tested against an isolated form of the potential target, bovine brain tubulin.

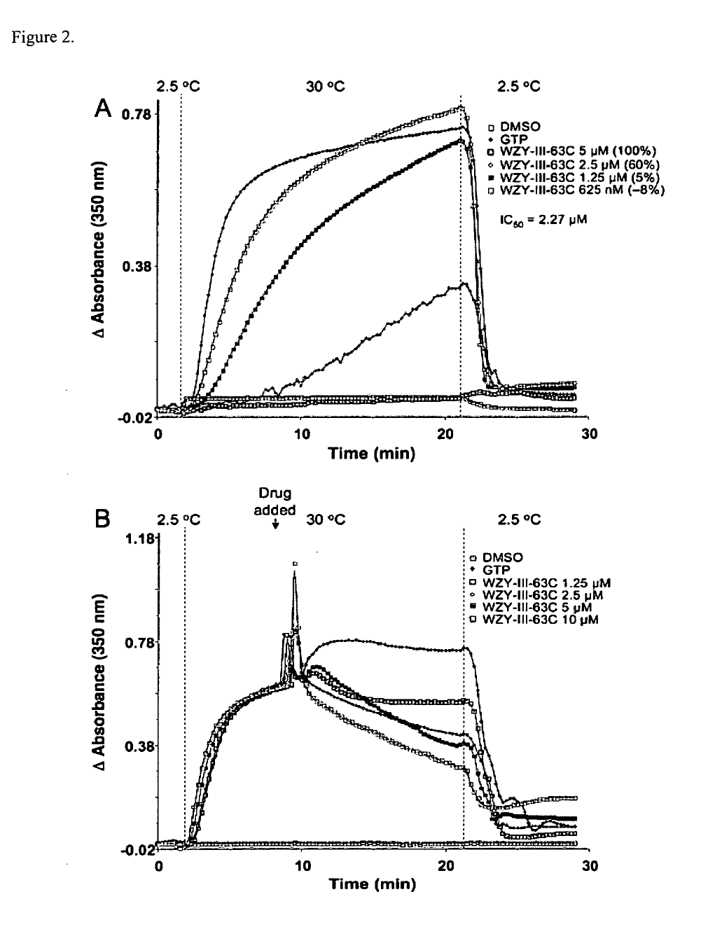

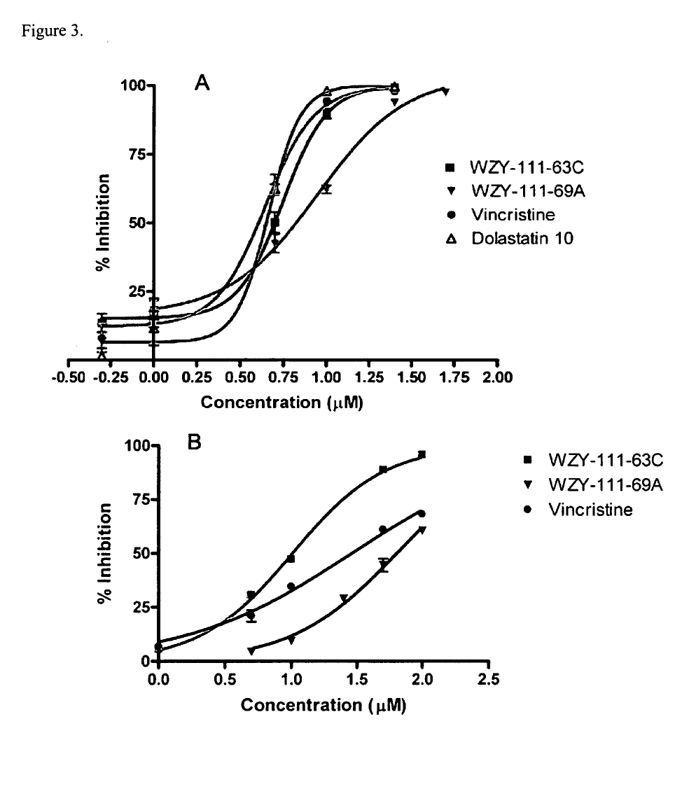

In turbidimetry experiments, it was found that the ability to inhibit the GTP-induced assembly of tubulin decreased in the series WZY-III-63C>WZY-III-69A>>WZY-III-64A (Table 3, FIG. 2A). Since tubulysin A has been reported to destabilize microtubules, WZY-III-63C was tested in a variant of this assay for its ability to cause disassembly of preformed microtubules (FIG. 2B). The ability to inhibit binding of radiolabeled versions of a vinca alkaloid and the peptide dolastatin 10 to tubulin was also examined (Table 3, FIG. 3). Again, the potency decreased in the series WZY-III-63C>WZY-III-69A>>WZY-III-64A. In all assays, WZY-III-63C showed potencies on par with the vinca alkaloids examined.

TABLE III^{a}
High-content analysis of mitotic arrest in HeLa cells
treated with antimitotic agents

| | | Nuclear | Microtubule | Mitotic |
|---|---|---|---|---|
| | Cell Density | Condensation | Density | Index^{c} |
| | EC₅₀ (nM) | MDEC (nM)^{b} | | |
| paclitaxel | 11.3 ± 2.7 | 10.2 ± 8.2 | 7.0 ± 2.9 | 4.2 ± 3.6 |
| vincristine | 19.7 ± 10.7 | 2.6 ± 2.0 | 10.5 ± 10.1 | 4.1 ± 1.9 |
| WZY-III- | 17.5 ± 6.6 | 12.4 ± 10.2 | 15.1 ± 11.1 | 2.5 ± 0.4 |
| 69A | | | | |
| WZY-III- | 95.7 ± 20.8 | 32.4 ± 8.6 | 56.6 ± 30.9 | 23.9 ± 5.8 |
| 64A | | | | |
| WZY-III- | 0.37 ± 0.09 | 0.13 ± 0.04 | 0.16 ± 0.05 | 0.10 ± 0.02 |
| 63C | | | | |

^{a}Average of four independent experiments ± SD.
^{b}Minimum detectable effective concentration.
^{c}Percentage of phosphohistone H3-positive cells

TABLE IV
Effects of tubulysin analogs on the proliferation of T98G glioblastoma
cells, tubulin assembly, and the binding of vinca domain and
peptide/depsipeptide site radiotracers

| | Antiproliferative | | Effects on Isolated Tubulin | |
|---|---|---|---|---|
| | Effects | | % Inhibition^{c} of | % Inhibition^{c} of |
| | | Tubulin | [³H]vinblastine binding | [³H]dolastatin 10 binding |
| | GI₅₀^{a} at 96 h (72 h) in | assembly | by 5 μM test agent and | by 5 μM test agent and test |
| | T98 glioblastoma cells | inhibition, | test agent | agent |
| | (nM) | IC₅₀ (μM)^{b} | IC₅₀^{d} | IC₅₀^{d} |
| WZY-III-63C | 1.6 ± 0.8 | 2.3 | 50 ± 4% | 31 ± 3% |
| (N¹⁴-desacetoxy- | (4.4 ± 11) | | 5.6 ± 0.5 μM | 10 ± 1 μM |
| tubulysin H) | | | | |
| WZY-III-64A | 400 ± 30 | >5 | 2 ± 5% | −11 ± 10% |
| | (590 ± 1000) | | >50 μM | ND^{e} |
| WZY-III-69A | 350 ± 40 | 3.1 | 43 ± 1% | 4 ± 3% |
| | (430 ± 130) | | 8.8 ± 1.6 μM | 64 ± 9 μM |
| paclitaxel | 12 ± 7 | N/A^{f} | N/A^{f} | N/A^{f} |
| | (24 ± 9) | | | |
| vinblastine | 1.0 ± 0.6 | 1.6 | N/A^{f} | 5 ± 6% |
| | (8.6 ± 0.9) | | | |
| dolastatin 10 | 0.056 ± 0.025 | 1.4 | 61 ± 1% | N/A^{f} |
| | (0.1 ± 0.02) | | 4.6 ± 0.5 μM | |
| vincristine | ND^{e} | ND | 65 ± 2% | 21 ± 5% |
| | | | (@50 μM: 98 ± 1%) | 29 ± 5 μM |
| | | | 4.4 ± 0.7 μM | |
| colchicine | ND^{e} | ND | −11 ± 12% | −5 ± 8% |

^{a}Fifty percent growth inhibitory concentration (±SD; n = 4 for each data point).
^{b}Concentration of test agent necessary to cause, after 20 min at 30° C., a 50% decrease in GTP-induced assembly of 10 μM bovine brain tubulin in 0.8 M monosodium glutamate.
^{c}Determined after a 30 min room temperature incubation with 5 μM test agent, 10 μM radiotracer and 10 μM bovine brain tubulin in 0.1 M 2-(4-morpholino)ethanesulfonic acid, pH 6.9, containing 0.5 mM MgCl₂ by centrifugal gel filtration and protein concentration determination. Inhibition is the percent difference relative to that of the control incubation with DMSO (±SD; n = 4 for each data point).
^{d}Performed as in ^{c}, except a range of test agent concentrations (0.5-100 μM) was employed; GraphPad Prism was used to fit the data to the Hill equation to determine the IC₅₀ value (±SD; n = 4 for each data point).
^{e}Not determined.
^{f}Not applicable.

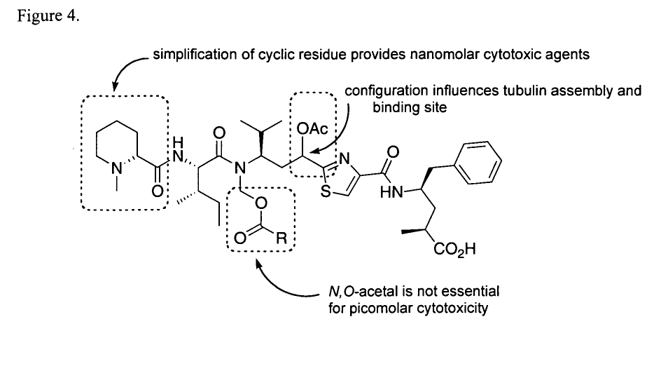

The above-described synthesis and biological evaluation of N¹⁴-desacetoxytubulysin H (WZY-III-63C), WZY-III-64A, and WZY-III-69A establish a SAR for compounds of the present invention (see FIG. 4), namely:

The labile N, O-acetal at N¹⁴ is not essential for biological activity. While the N-methyl compound WZY-III-63C (N¹⁴-desacetoxytubulysin H) loses 1-2 orders of magnitude in the antiproliferative GI₅₀ compared to dolastatin 10 and tubulysins A or D, it is still a picomolar cytotoxic agent and profoundly perturbs microtubule assembly in vitro and in intact cells. Furthermore, WZY-III-63C is equipotent to dolastatin 10 in its ability to inhibit [³H]vinblastine binding to isolated tubulin.

The replacement of the N-terminal Mep residue with the achiral building block N-methylsarcosine leads to a more significant loss of activity, but still provides agents with nanomolar biological potencies.

The natural (R)-configuration of the acetate at C¹¹ is preferred with regard to effects on isolated tubulin, but the differences in the GI₅₀ values in T98 glioblastoma cells are minor between the diastereomers WZY-III-64A and WZY-III-69A.

Accordingly, the present invention empowers the modulation of biological activity gradually in this context, with an apparent slight variation of the mode of action of the derivatives, thereby informing the generation of structurally simplified antitubulin agents based on the tubulysin scaffold. In accordance with the invention, moreover, there is an opportunity to tune the selectivity of the tubulin scaffold toward more cancer-selective toxicity.

### EXAMPLE 20

### Simplification of Tubulysin Scaffold and Synthesis of Inventive Compounds

Pursuant to the aforementioned SAR, a synthesis can be undertaken, depicted in Scheme 6, to prepare a compound 9, as shown. The alpha-stereogenic center in tubuphenylalanine (Tup) is removed by the incorporation of a double bond in the molecule. Thus, aldehyde 1 is coupled with the thiazole anion from 2, and the resulting mixture of alcohol epimers is oxidized by Dess-Martin periodinane to give ketone 3. CBS-mediated asymmetric reduction then gives 4, which is acylated, deprotected, oxidized, and coupled with amine 6 to give dipeptide 7. Successive coupling with Fmoc-Ile-F and Mep-pentafluorophenyl ester, followed by final deprotection of the allyl group, would furnish 9 (Scheme 6).

To simplify the structure of N¹⁴-desacetoxytubulysin H further, compounds 14, 17 and 19 are designed by removing the labile acetate in tubuvaline (Tuv). As shown in Scheme 7, alkyne 10 is coupled with thiazole 11 to give 12, which is saponified into the corresponding thiazole acid and coupled, similarly as above, to give analog 14. A similar process would use 15 instead of 10 to couple with 11 under Negishi conditions, and 16 would be elaborated into analog 17. The double bond in 16 also could be hydrogenated to give 18, which would be used to synthesize analog 19 (Scheme 7).

In an alternate embodiment compound 14 is prepared via a synthetic route shown in Scheme 8. Trimethylsilylacetylide 21 is selectively coupled to 20 using trimethylaluminum as a catalyst to give 22, which is methylated to give 23. Acid catalyzed removal of the tert-butylsulfinyl group from 23 followed by coupling with Fmoc-Ile-F and removal of the Fmoc and trimethylsilyl (TMS) protecting groups results in 24, which is coupled with D-Mep-pentafluorphenyl ester to give 25. Coupling of alkyne 25 to 26 under Sonogashira conditions results in compound 28. Alternatively, coupling of alkyne 25 to 27 under Sonogashira conditions followed by palladium catalyzed deprotection the terminal allyl group resulted in 14 (Scheme 8).

Scheme 9 provides an alternate synthetic route for synthesizing 19. As shown in Scheme 9, alkyne 23 is deprotected to remove the trimethylsilyl and tert-butylsulfinyl protecting groups. The deprotected alkyne is reacted with di-t-butyloxycarbonyl carbonate under basic conditions to give 29, which is coupled to ethyl-2-bromothiazole-4-carboxylate 30 under Sonogashira conditions to give 31. Hydrogenation of 31 gives the saturated analog 32 which upon saponification followed by coupling to ethyl-4-amino-2-methyl-5-phenyl pent-2-eneoic acid 33 gives 34. Successive coupling of 34 with Fmoc-Ile-F followed by Mep-pentafluorophenyl ester, and saponification of the terminal ethyl ester results in 19.

A similar strategy can be used to synthesize compounds 37-39 whose structures are shown in Scheme 10.

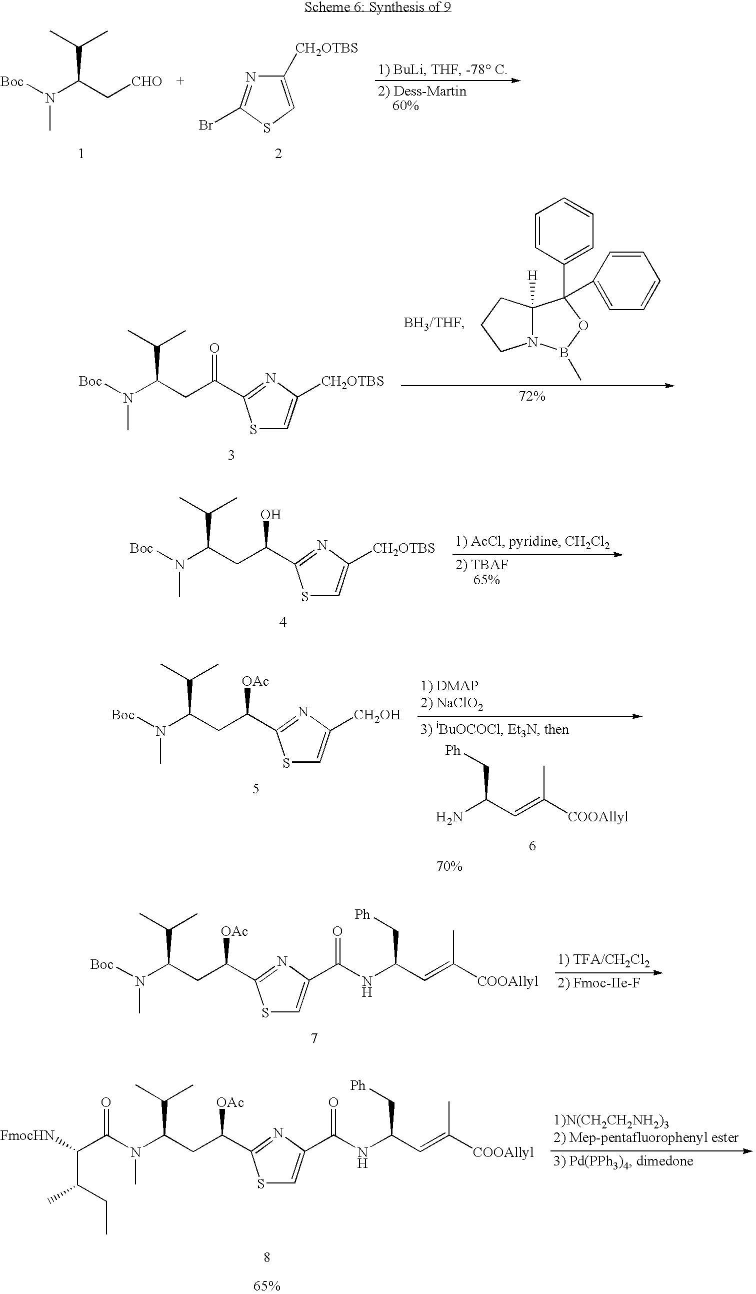

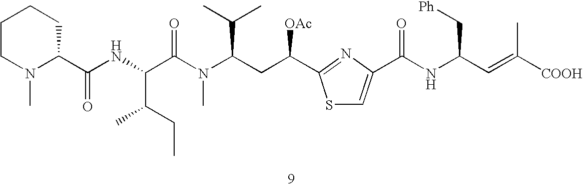

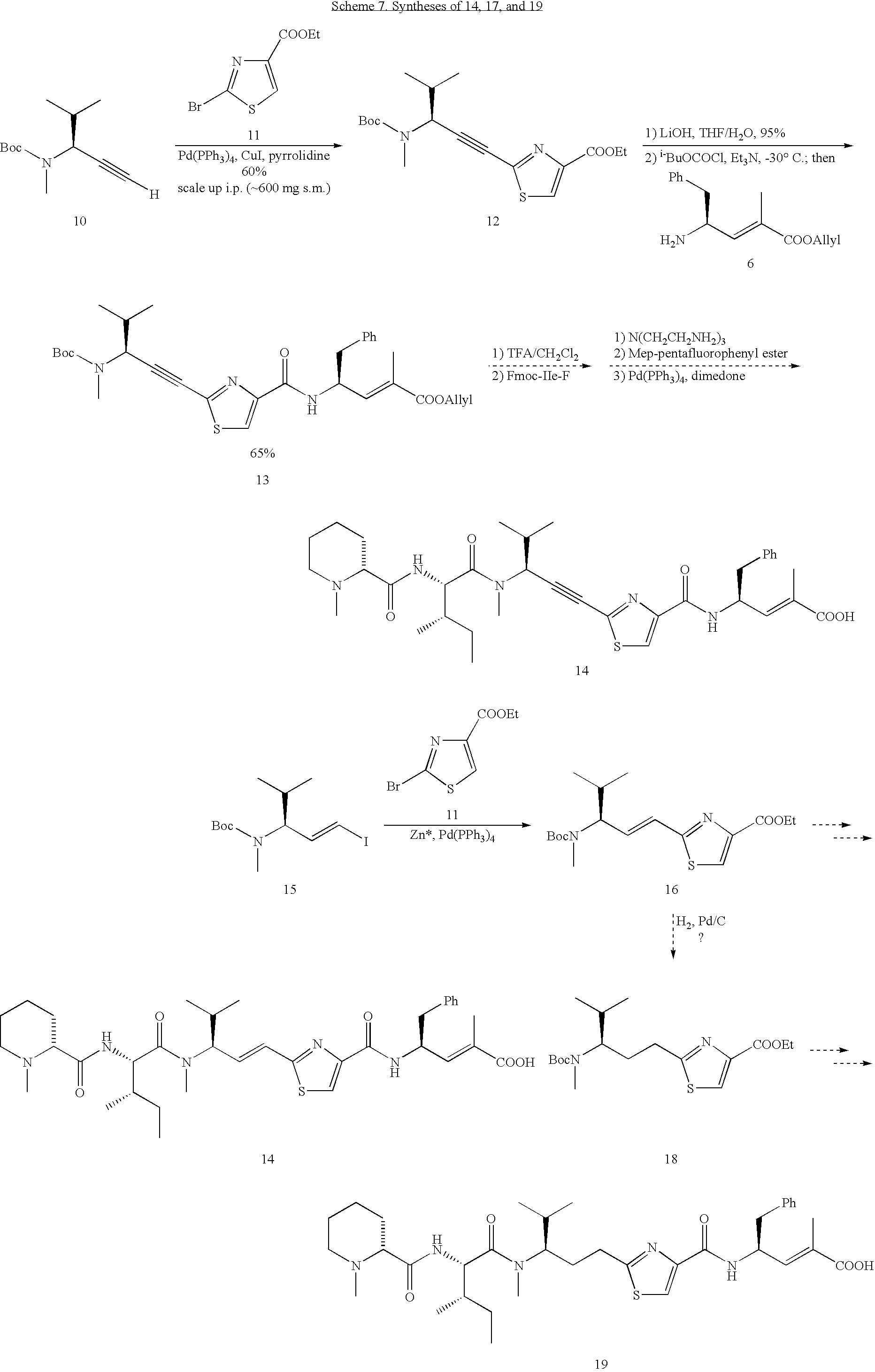

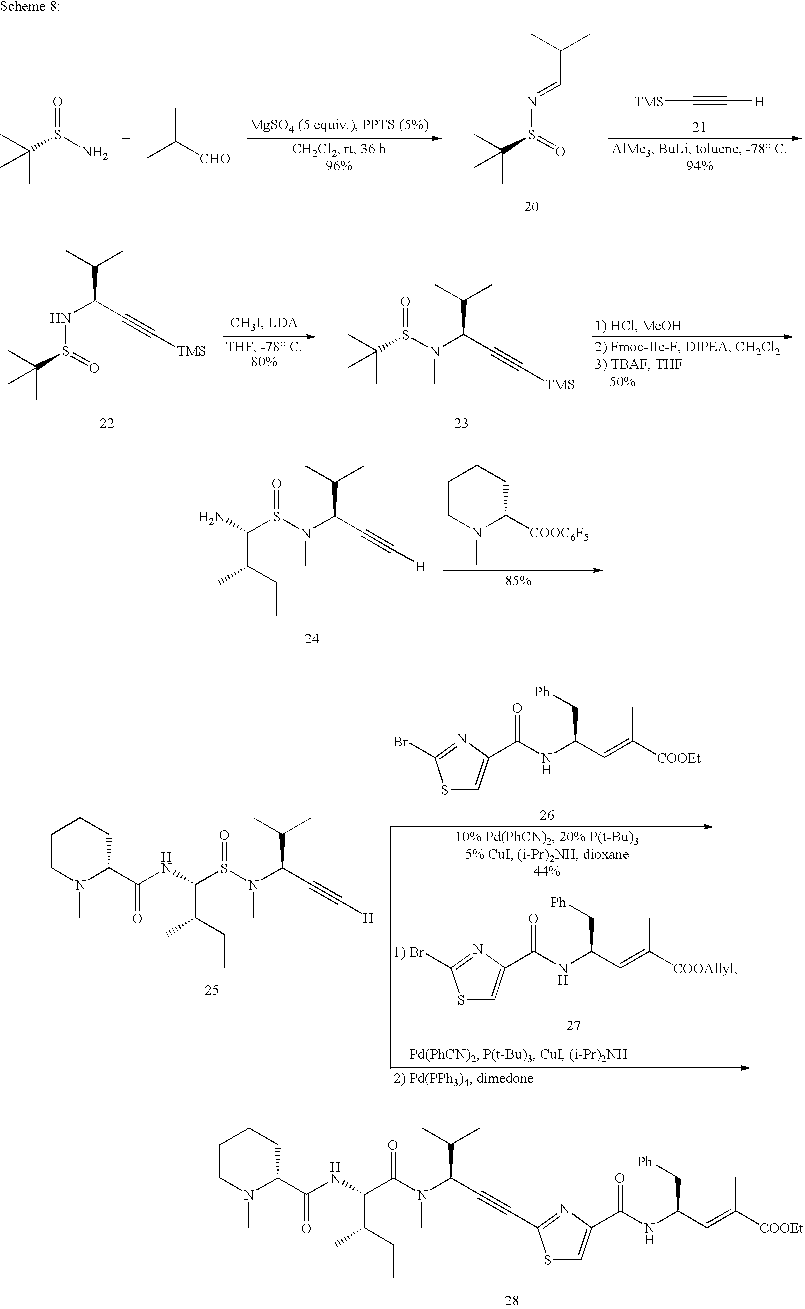

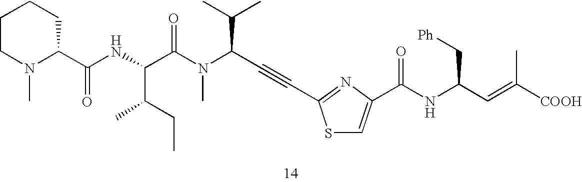

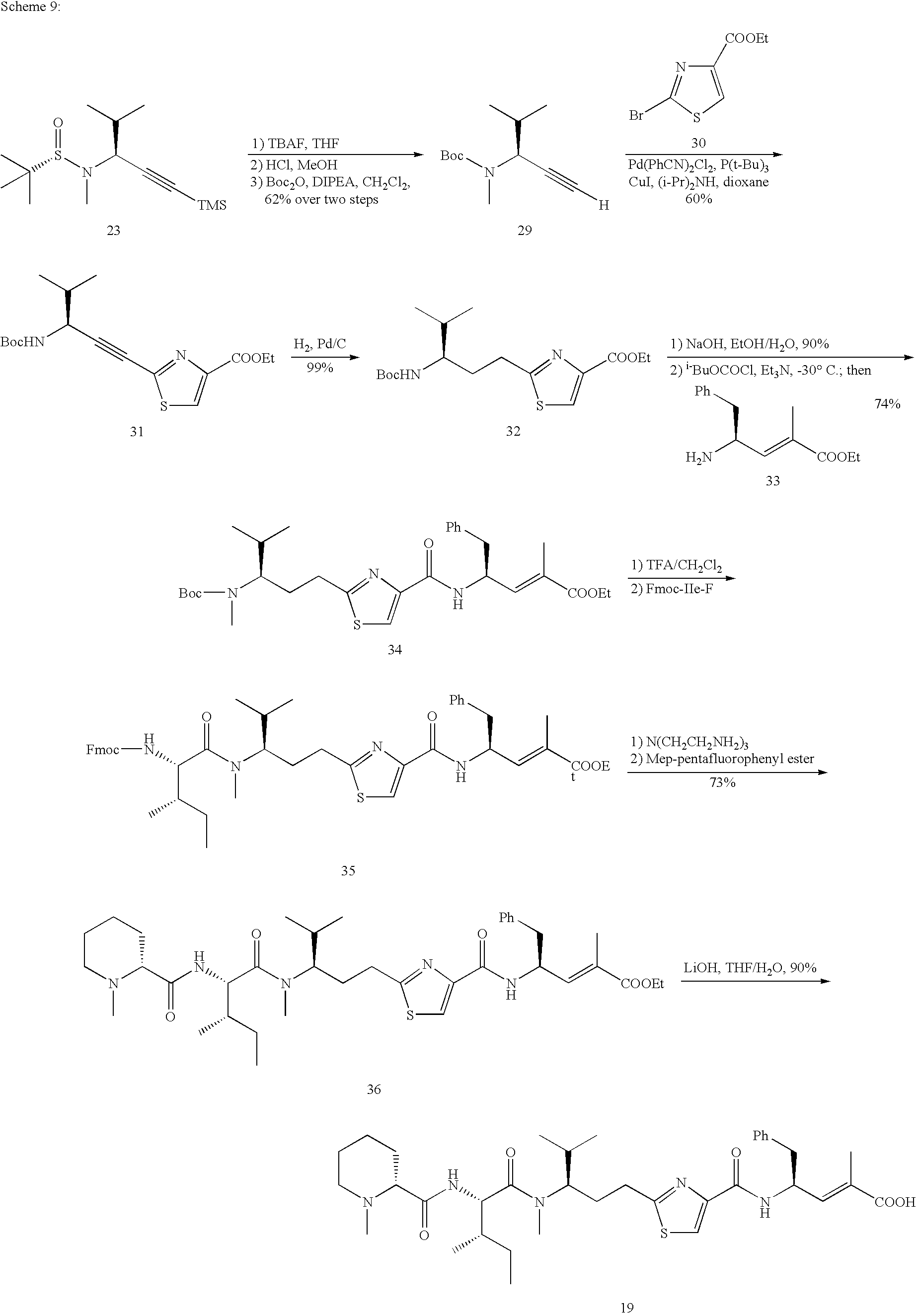

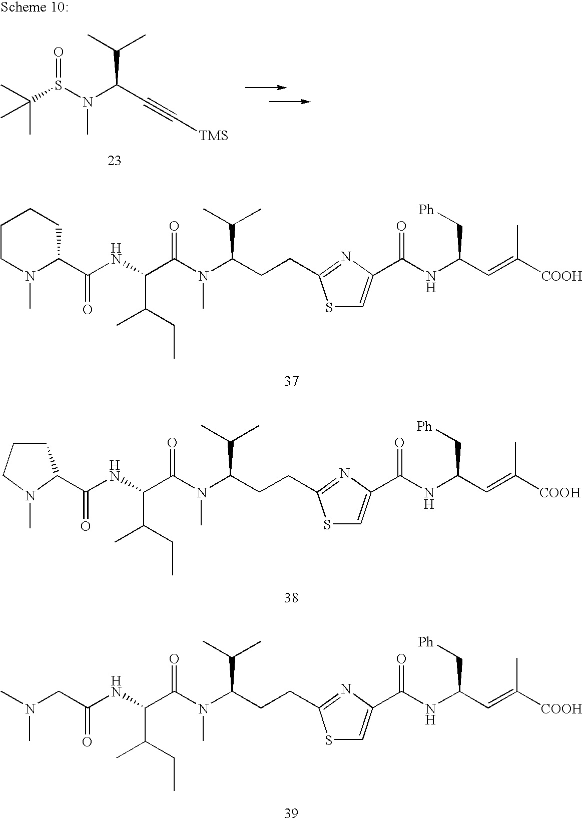

### EXAMPLE 21

### (S,E-2-Methyl-N-(2-methylpropylidene)propane-2-sulfinamide (20)

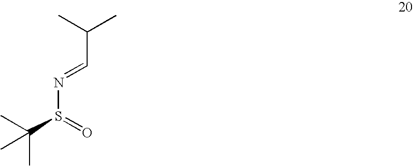

To a suspension of (S)-(−)-tert-butanesulfinamide (4.70 g, 38.78 mmol), magnesium sulfate (23.3 g, 193.57 mmol) and pyridinium p-toluenesulfonate (0.50 g, 1.95 mmol) in anhydrous dichloromethane (75 mL) was added isobutyraldehyde (distilled (bp 63-64° C.) from anhydrous CaSO₄ under N₂ protection 13.0 mL, 140.29 mmol), and the mixture was stirred at room temperature for 36 h. The mixture was filtered over a pad of Celite, concentrated, and chromatographed (EtOAc/hexanes, 1:10 to 1:3) to give 20 (6.55 g, 96%) as a colorless oil; R_{f}=0.50 (EtOAc/hexanes, 1:); [α]_{D}²³=+278.4 (c 1.12, CH₂Cl₂) (lit. [α]_{D}²³=−259.4 (c 1.0, CHCl3) for the enantiomer); ¹H NMR (CDCl₃, 600 MHz) δ 7.98 (d, 1H, J=4.2 Hz), 2.75-2.67 (m, 1 H), 1.18 (s, 9H), 1.17 (d, 3H, J=6.6 Hz), 1.16 (d, 3H, J=6.6 Hz); ¹³C NMR (CDCl₃, 150 MHz) δ 173.5, 56.4, 34.8, 22.3, 18.9; IR 2964, 1620, 1458, 1363, 1083 cm⁻¹. Chiral HPLC analysis (Daicel Chiralcel OD silica gel HPLC column, 250×4.6 mm; isocratic elution with 90% isopropanol in hexanes, 0.5 mL/min; detection at 254 nm) gave a single peak at 7.84 min, and e.e. was determined to be >99%. (Note: Analysis of a racemic sample showed the enantiomer peak at 8.60 min.)

### EXAMPLE 21

### (S)-2-Methyl-N—((S)-4-methyl-1-(trimethylsilyl)pent-1-yn-3-yl)propane-2-sulfinamide (22)

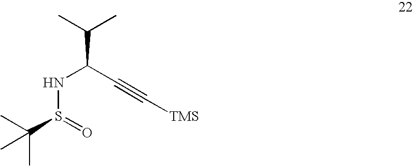

To a solution of trimethylsilylacetylene (9.86 g, 98.38 mmol) in anhydrous toluene (˜160 mL) cooled in a dry ice/acetone bath was added a solution of butyllithium in hexanes (1.30 M, 54.0 mL, 70.20 mmol) dropwise over 20 min, and the mixture was stirred for 30 min before a cooled solution (−78° C.) of 20 (6.78 g, 38.68 mmol) and trimethylaluminum (2.0 M solution in toluene, 23.0 mL, 46.0 mmol) in toluene (˜40 mL) was cannulated dropwise (40 min). The mixture was allowed to gradually warm up to room temperature overnight, and it was cooled in an ice bath and carefully quenched with water (30 mL) (CAUTION: gas-forming and exothermic) and partitioned between ethyl acetate (50 mL) and hydrochloric acid (1 N, 150 mL). The aqueous phase was extracted with ethyl acetate (60 mL×2), and the organics were combined, washed with saturated sodium bicarbonate (10 mL) and brine (10 mL), dried (Na₂SO₄), evaporated and chromatographed (EtOAc/hexanes, 1:10 to 1:5) to give 22 (10.0 g, 94%) as a colorless oil; [α]_{D}²³=+33.4 (c 1.10, CH₂Cl₂); R_{f}=0.26 (EtOAc/hexanes, 1:3, I₂ staining); ¹H NMR (CDCl₃, 600 MHz) δ 3.91 (t, 1H, J=5.4 Hz), 3.28 (d, 1H, J=5.4 Hz), 1.94-1.86 (m, 1H), 1.21 (s, 9H), 0.98 (d, 3H, J=6.6 Hz), 0.96 (d, 3H, J=6.6 Hz), 0.15 (s, 9H); ¹³C NMR (CDCl₃, 150 MHz) δ 103.7, 90.2, 56.1, 53.8, 33.3, 22.5, 18.9, 17.0, −0.2; IR (film) 3208, 2960, 2901, 2173, 1468, 1249, 1058, 840; HRMS (ESI) calcd for C₁₃H₂₇NOSSiNa ([M+Na]⁺) 296.1480. found 296.1472. Diastereomeric excess (d.e.) was determined to be 98.7% based on ¹H NMR integration (δ 3.28, J=6.0 Hz for the NH proton; δ 3.22, J=6.6 Hz for the NH of the minor diastereomer). The material partially crystallized to give colorless crystals (mp 57-59° C.) that were suitable for X-ray analysis upon standing at room temperature, and the desired stereochemistry was confirmed by X-ray crystallography.

### (S)—N,2-Dimethyl-N—((S)-4-methyl-1-(trimethylsilyl)pent-1-yn-3-yl)propane-2-sulfinamide (23)

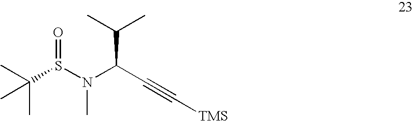

To a solution of diisopropylamine (2.50 mL, 17.51 mmol) in anhydrous THF (24 mL) cooled at −78° C. was added dropwise butyllithium (1.1 M solution in hexanes, 10.0 mL, 11.0 mmol) followed by HMPA (0.84 mL, 4.78 mmol), and the solution was stirred further for 10 min (green solution) before a solution of 22 (1.18 g, 4.31 mmol) in THF (10 mL) was added via cannula dropwise (yellow solution). The solution was stirred further for 20 min, and iodomethane (2.70 mL, 42.94 mmol) was added dropwise. The solution was stirred further at −78° C. for 4 h, and the reaction was then quenched with water (20 mL) and most of the THF was evaporated under reduced pressure. The residue was partitioned between ethyl acetate (80 mL) and hydrochloric acid (1 N, 20 mL), and the separated organic phase was washed with saturated sodium bicarbonate (20 mL), sodium thiosulfate (1 M, 20 mL) and brine (20 mL), dried (Na₂SO₄), concentrated, and chromatographed (EtOAc/hexanes, 1:7 to 1:5) to give 23 (0.99 g, 80%) as a colorless oil; R_{f}=0.40 (EtOAc/hexanes, 1:3, I₂ staining); [α]_{D}²³=−136.7 (c 1.44, CH₂Cl₂); ¹H NMR (CDCl₃, 600 MHz) δ 3.66 (d, 1 H, J=10.2 Hz), 2.63 (s, 3H), 1.93-1.85 (m, 1H), 1.20 (s, 9H), 1.06 (d, 3H, J=7.2 Hz), 0.94 (d, 3H, J=6.6 Hz), 0.16 (s, 9H); ¹³C NMR (CDCl₃, 150 MHz) δ 103.7, 90.6, 59.4, 31.4, 28.4, 23.2, 20.0, 19.6, −0.1; IR (film) 2960, 2872, 2166, 1467, 1249, 1084, 1022, 840, 759 cm⁻¹; HRMS (ESI) calcd for C₁₄H₂₉NOSSiNa ([M+Na]⁺) 310.1637. found 310.1616.

### (2S,3S)-2-Amino-N,3-dimethyl-N—((S)-4-methylpent-1-yn-3-yl)pentanamide (24)

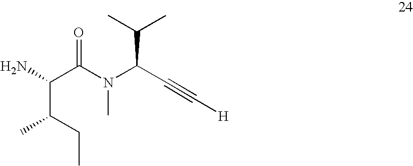

To a solution of 23 (0.264 g, 0.92 mmol) in methanol (10 mL) was added hydrogen chloride in methanol (1.25 M, 2.2 mL, 2.75 mmol), and the mixture was stirred at room temperature for 1 h. TLC analysis (EtOAc/hexanes, 1:3) showed only a baseline spot. The solution was evaporated under reduced pressure to give the crude amine hydrochloride (0.23 g, 100%) as a white solid, which was used without further purification.

To an ice bath-cooled solution of the above amine chloride (0.23 g) in anhydrous dichloromethane (10 mL) was added DIPEA (0.35 mL, 2.10 mmol) followed by Fmoc-Ile-F (0.365 g, 1.03 mmol), and the yellow solution was warmed up to room temperature and stirred overnight. TLC analysis showed two close spots, indicating that partial desilylation was effected by the fluoride salt that was generated. The mixture was diluted in EtOAc (30 mL), washed with saturated NaHCO₃ (10 mL) and brine (10 mL), dried (Na₂SO₄), evaporated, and chromatographed (EtOAc/hexanes, 1:10 to 1:5) to give two major fractions of colorless oil. The first (0.33 g, 70%) was mostly the desired product. The central pure fraction (single spot by TLC) was used for analysis: R_{f}=0.54 (EtOAc/hexanes, 1:3); [α]_{D}²³=−61.2 (c 1.11, CH₂Cl₂); ¹H NMR analysis showed a 16:1 mixture of rotamers at room temperature; Major rotamer: ¹H NMR (CDCl₃, 600 MHz) δ 7.77 (d, 2H, J=7.8 Hz), 7.59 (d, 2H, J=7.8 Hz), 7.40 (t, 2H, J=7.5 Hz), 7.31 (dt, 2H, J=7.6, 8.6 Hz), 5.50 (d, 1H, J=9.6 Hz), 5.11 (d, 1H, J=10.2 Hz), 4.54 (t, 1H, J=9.6 Hz), 4.36 (d, 2H, J=7.2 Hz), 4.22 (t, 1H, J=7.5 Hz), 3.09 (s, 3H), 1.92-1.77 (m, 2H), 1.65-1.58 (m, 1H), 1.19-1.14 (m, 1H), 1.08 (d, 3H, J=6.6 Hz), 0.98 (d, 3H, J=6.6 Hz), 0.93 (t, 3 H, J=7.5 Hz), 0.82 (d, 3H, J=6.6 Hz), 0.17 (s, 9H); ¹³C NMR (CDCl₃, 150 MHz) δ 172.3, 156.4, 143.9, 143.8, 141.3, 127.6, 127.0, 125.1, 119.9, 103.0, 89.5, 67.0, 55.2, 53.2, 47.2, 38.1, 31.0, 30.8, 24.2, 21.5, 19.5, 18.7, 15.4, 11.2, −0.1; Characteristic peaks of the minor rotamer: ¹H NMR (CDCl₃, 600 MHz) δ 2.94 (s, 3H), 0.10 (s, 9 H); ¹³C NMR (CDCl₃, 150 MHz) δ171.2, 155.9, 141.2, 125.1, 119.9, 101.7, 90.5, 23.7, 21.0, 14.2; IR (film) 3288, 2962, 2936, 2875, 2175, 1714, 1634, 1527, 1450, 1408, 1248, 1034, 841, 758, 739 cm⁻¹; HRMS (ESI) calcd for C₃₁H₄₂N₂O₃NaSi ([M+Na]⁺) 541.2862. found 541.2841. The second (0.127 g, 31%) was mostly the corresponding desilylated product. All the fractions were combined (0.46 g) treated with TBAF.

To a solution of the above dipeptide (0.46 g, ˜0.92 mmol) in THF (5 mL) was added tetrabutylammonium fluoride (1.0 M solution in THF, 1.0 mL, 1.0 mmol), and the solution was stirred at room temperature for 3 h, diluted in ethyl acetate (40 mL), washed with saturated sodium bicarbonate (10 mL) and brine (10 mL), dried (Na₂SO₄), evaporated, and chromatographed (EtOAc/hexanes, 1:1 followed by 3% MeOH and 1% Et₃N in CH₂Cl₂) to give 24 (0.206 g, 50% purity, 50% yield for three steps) as a ˜1:1 mixture of the desired amine and tetrabutylammonium fluoride; R_{f}=0.33 (5% MeOH in CH₂Cl₂, I₂ staining); ¹H NMR (CDCl₃, 600 MHz) δ 5.17 (dd, 1H, J=9.9, 2.1 Hz), 3.56-3.55 (m, 1H), 3.03 (s, 3H), 2.27 (d, 1H, J=2.4 Hz), 2.01-1.93 (m, 4H), 1.10 (d, 3H, J=6.6 Hz), 1.02 (t, 3H, J=7.8 Hz), 0.96 (d, 3H, J=7.8 Hz), 0.87 (d, 3H, J=6.6 Hz); MS (ESI) 225 ([M+H]⁺).

### (R)-1-Methyl-N-((2S,3S)-3-methyl-1-(methyl((S)-4-methylpent-1-yn-3-yl)amino)-1-oxopentan-2-yl)piperidine-2-carboxamide (25)

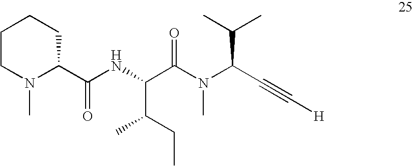

To a solution of N-methyl D-pipecolic acid (88 mg, 0.62 mmol) and pentafluorophenol (0.14 g, 0.77 mmol) in anhydrous DMF (1.0 mL) was added EDC hydrochloride (0.14 g, 0.73 mmol), and the solution was stirred at room temperature for overnight to give a yellow clear solution, which was directly used in the following coupling reaction.

The active ester in anhydrous DMF prepared as above was then added to a solution of 24 (0.20 g, 50% purity, 0.446 mmol) in DMF (0.7 mL), and the solution was stirred at room temperature for 8 h. The mixture was dissolved in EtOAc (60 mL), washed with sodium hydroxide (1 N, 10 mL) and brine (10 mL), dried (Na₂SO₄), evaporated, and chromatographed (EtOAc/hexanes, 1:3; followed by 2% MeOH in CH₂Cl₂ to elute the product) to give 25 (0.132 g, 85%) as a yellow oil; [α]_{D}²³=−19.0 (c 1.50, CH₂Cl₂); R_{f}=0.31 (5% MeOH in CH₂Cl₂); ¹H NMR (CDCl₃, 600 MHz) δ 7.04 (d, 1H, J=9.0 Hz), 5.10 (dd, 1H, J=10.2, 2.4 Hz), 4.72 (t, 1H, J=9.0 Hz), 3.14 (s, 3H), 2.91-2.89 (m, 1H), 2.48 (dd, 1H, J=11.4, 3.0 Hz), 2.27 (d, 1H, J=2.4 Hz), 2.23 (s, 3H), 2.02 (dt, 1H, J=11.7, 2.6 Hz), 1.92-1.85 (m, 2H), 1.79-1.77 (m, 1H), 1.68 (dt, 1H, J=12.6, 3.3 Hz), 1.62-1.51 (m, 3H), 1.37 (dq, 1H, J=13.0, 4.2 Hz), 1.25-1.14 (m, 2H), 1.08 (d, 3H, J=7.2 Hz), 0.95 (d, 3H, J=7.8 Hz), 0.90 (t, 3H, J=7.2 Hz), 0.78 (d, 3H, J=7.2 Hz); ¹³C NMR (CDCl₃, 150 MHz) δ 174.3, 172.2, 81.3, 72.4, 69.6, 55.5, 52.6, 52.0, 44.8, 37.3, 31.1, 30.9, 30.3, 25.0, 24.8, 23.2, 19.4, 18.4, 15.4, 10.8; IR (film) 3311, 3238, 2963, 2936, 2790, 2112, 1639, 1501, 1464 cm⁻¹; MS (ESI) 350 ([M+H]⁺); HRMS (ESI) calcd for C₂₀H₃₆N₃O₂ ([M+H]⁺) 350.2808. found 350.2807.

### (S,E)-Ethyl 4-(2-((S)-3-((2S,3S)—N,3-dimethyl-2-((R)-1-methylpiperidine-2-carbox-amido)pentanamido)-4-methylpent-1-ynyl)thiazole-4-carboxamido)-2-methyl-5-phenylpent-2-enoate (28)

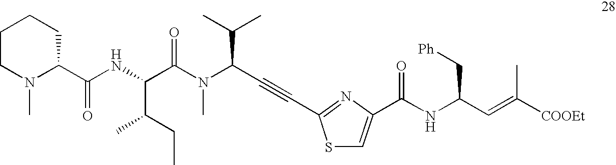

To a mixture of 25 (46 mg, 0.132 mmol), 26 (75 mg, 0.177 mmol), and tri-tert-butylphosphine (10 wt % solution in hexane, 54 mg, 0.027 mmol) in anhydrous 1,4-dioxane (0.4 mL) was added bis(benzonitrile)palladium(II) chloride (6 mg, 0.015 mmol) followed by diisopropylamine (0.05 mL, 0.350 mmol) and copper(I) iodide (1.3 mg, 0.0066 mmol). The mixture was degassed by the freeze-pump-thaw method and stirred at room temperature for 30 h to give a dark suspension, which was diluted in ethyl acetate (60 mL), washed with hydrochloric acid (1 N, 10 mL), saturated sodium bicarbonate (10 mL) and brine (10 mL), dried (Na₂SO₄), concentrated, and chromatographed (EtOAc/hexanes, 1:3 to 1:1; followed by 3% then 5% MeOH in CH₂Cl₂) to give 28 (40 mg, 44%) as a yellow oil; [α]_{D}²³=+6.8 (c 1.00, CH₂Cl₂); R_{f}=0.38 (CH₂Cl₂/MeOH, 95:5); ¹H NMR analysis at room temperature showed an 11:1 mixture of rotamers. Major rotamer: ¹H NMR (CDCl₃, 600 MHz) δ 8.07 (s, 1H), 7.38 (d, 1H, J=8.4 Hz), 7.30-7.22 (m, 5H), 7.03 (d, 1H, J=8.4 Hz), 6.63 (dd, 1H, J=9.0, 1.2 Hz), 5.43 (d, 1H, J=10.2 Hz), 5.15 (quintet, 1H, J=8.1 Hz), 4.76 (t, 1 H, J=9.0 Hz), 4.21-4.16 (m, 2H), 3.24 (s, 3H), 3.10 (dd, 1H, J=13.2, 10.0 Hz), 2.92 (dd, 2H, J=13.8, 7.8 Hz), 2.49 (dd, 1H, J=10.8, 2.4 Hz), 2.24 (s, 3H), 2.08-2.00 (m, 2H), 1.93-1.88 (m, 1H), 1.80 (d, 1H, J=13.2 Hz), 1.74 (d, 3H, J=1.2 Hz), 1.71-1.52 (m, 4H), 1.40-1.34 (m, 2H), 1.29 (t, 3H, J=6.9 Hz), 1.17 (d, 3H, J=6.6 Hz), 0.99 (d, 3H, J=7.2 Hz), 0.92 (t, 3H, J=7.5 Hz), 0.86 (d, 3H, J=7.2 Hz); ¹³C NMR (CDCl₃, 150 MHz) δ 174.5, 172.6, 167.6, 159.5, 150.1, 147.9, 138.7, 136.5, 130.6, 129.5, 128.5, 126.8, 124.9, 93.6, 69.6, 60.8, 55.4, 52.7, 52.6, 49.0, 44.9, 41.0, 37.2, 31.3, 31.1, 30.4, 29.7, 25.1, 24.8, 23.3, 19.7, 18.5, 15.6, 14.1, 12.7, 10.9. Characteristic peaks of the minor rotamer: ¹H NMR (CDCl₃, 600 MHz) δ 8.05 (s, 1 H). IR (film) 3317, 2963, 2936, 2874, 2234, 1712, 1648, 1533, 1477, 1256 cm⁻¹; MS (ESI) 692 ([M+H]⁺); HRMS (ESI) calcd for C₃₈H₅₄N₅O₅S ([M+H]⁺) 692.3846. found 692.3845.

All patents and other references cited in the specification are indicative of the level of skill of those skilled in the art to which the invention pertains, and are incorporated by reference in their entireties, including any tables and figures, to the same extent as if each reference had been incorporated by reference in its entirety individually.

All patents and other references cited in the specification are indicative of the level of skill of those skilled in the art to which the invention pertains, and are incorporated by reference in their entireties, including any tables and figures, to the same extent as if each reference had been incorporated by reference in its entirety individually.

One skilled in the art would readily appreciate that the present invention is well adapted to obtain the ends and advantages mentioned, as well as those inherent therein. The methods, variances, and compositions described herein as presently representative of preferred embodiments are exemplary and are not intended as limitations on the scope of the invention. Changes therein and other uses will occur to those skilled in the art, which are encompassed within the spirit of the invention, are defined by the scope of the claims.

It will be readily apparent to one skilled in the art that varying substitutions and modifications may be made to the invention disclosed herein without departing from the scope and spirit of the invention. For example, variations can be made to provide additional compounds of the invention, syntheses thereof, various methods of administration, and/or various indications of disease or other condition. Thus, such additional embodiments are within the scope of the present invention and the following claims.

The invention illustratively described here suitably may be practiced in the absence of any element or elements, limitation or limitations that is not specifically disclosed. Thus, in each instance in this description any of the terms “comprising,” “consisting essentially of” and “consisting of” may be replaced with either of the other two terms so as to provide additional embodiments of the invention. The terms and expressions that have been employed are used as terms of description and not of limitation, and there is no intention in the use of such terms and expressions of excluding any equivalents of the features shown and described or portions thereof, but it is recognized that various modifications are possible within the scope of the invention claimed. Accordingly, it should be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

In addition, where features or aspects of the invention are described in terms of Markush groups or other grouping of alternatives, those skilled in the art will recognize that the invention is also thereby described in terms of any individual member or subgroup of members of the Markush group or other group.

Also, unless indicated to the contrary, where various numerical values are provided for embodiments, additional embodiments are described by taking any two different values as the endpoints of a range. Such ranges are also within the scope of the described invention.

Thus, additional embodiments are within the scope of the invention and within the following claims.

What is claimed is:

1. A process for the preparation of a compound with structure of Formula I: 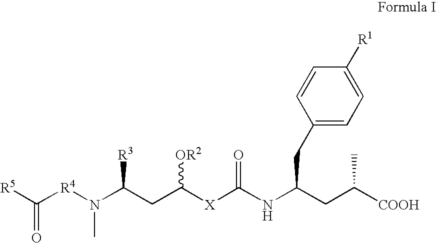
wherein:
R¹ is H or OH;
R² is H or C(O)R;
R³ is C₁₋₆ alkyl;
R⁴ is an amino acid selected from the group consisting of glycine, cysteine, alanine, histidine, asparagine, glutamine, arginine, threonine, valine, leucine, isoleucine, phenylalanine, tryptophan, serine, lysine, aspartic acid, methionine, glutamic acid, tyrosine, and optionally substituted derivatives thereof;
R⁵ is selected from the group consisting of optionally substituted heteroalkyl, optionally substituted heterocycloalkyl, and optionally substituted heteroaryl;
R⁶ is selected from the group consisting of H, optionally substituted alkyl, optionally substituted aryl, and optionally substituted heteroaryl; and
X is optionally substituted arylene or optionally substituted heteroarylene; said process comprising:
(a) condensing a first protected amino acid with structure of Formula II: 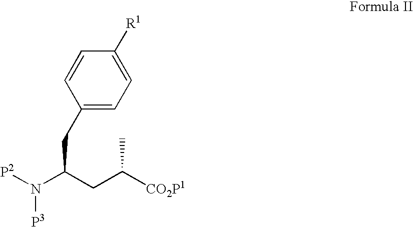
with a second protected amino acid with structure of Formula III: 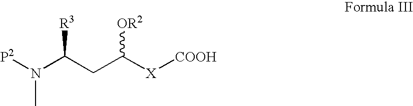
under conditions suitable to deprotect said first protected amino acid and to form a first compound with structure of Formula IV: 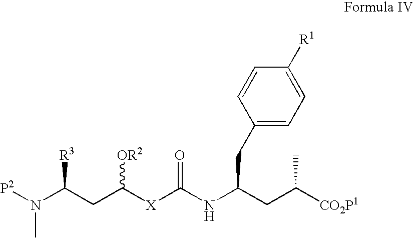
wherein:
P¹ is an acid protecting group;
P² at each occurrence is independently an amine protecting group; and
P³ is H or P²;
(b) reacting said first compound with a third protected amino acid with structure P²—R⁴ under conditions suitable to deprotect said first compound and to form a second compound with structure of Formula V: 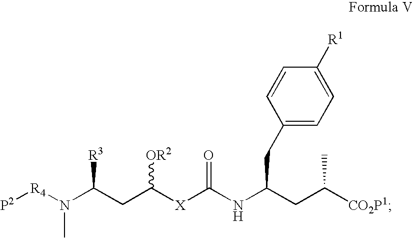
and
(c) reacting said second compound with a reagent with structure of Formula VI
R⁵—COOH Formula VI
or acid protected derivative thereof under conditions suitable to deprotect said second compound and to form said compound having structure of Formula I.

2. The process according to claim 1, wherein R¹ is H.

3. The process according to claim 1, wherein R¹ is OH.

4. The process according to claim 1, wherein R² is H.

5. The process according to claim 1, wherein R² is acetyl.

6. The process according to claim 1, wherein R³ is C₁₋₃ alkyl.

7. The process according to claim 1, wherein R³ is prop-2-yl.

8. The process according to claim 1, wherein R⁴ is an L-amino acid.

9. The process according to claim 1, wherein R⁴ is a D-amino acid.

10. The process according to claim 1, wherein R⁴ is L-isoleucine.

11. The process according to claim 1, wherein R⁵ is optionally substituted heteroalkyl.

12. The process according to claim 1,
wherein
R⁵—COOH is R⁷R⁸N—(CH₂)ₙ—COOH,
each of R⁷ and R⁸ are independently C₁₋₆ alkyl, or
R⁷ and R⁸, together with the nitrogen to which they are attached, form an optionally substituted 5-7 membered heterocycloalkyl or heteroaryl; and
n is 1 to 6.

13. The process according to claim 1, wherein R⁵—COOH is 2-(dimethylamino)acetic acid.

14. The process according to claim 1, wherein R⁵ is optionally substituted heterocycloalkyl.

15. The process according to claim 1, wherein R⁵—COOH is 1-methylpiperidine-2-carboxylic acid (Mep).

16. The process according to claim 1,
wherein
said second protected amino acid has the structure of Formula IIIₐ 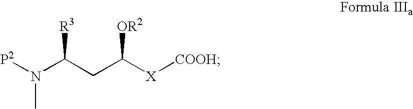
said first compound has the structure of Formula IVₐ 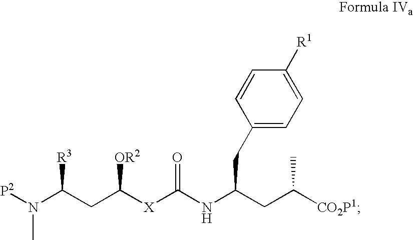
said second compound has the structure of Formula Vₐ 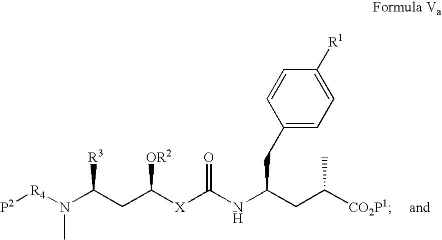
said reagent with structure of Formula VI is 1-methylpiperidine-2-carboxylic acid or acid protected derivative thereof; and
said compound with structure of Formula I has the structure of Formula Iₐ

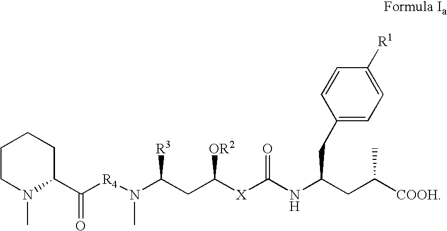

17. The process according to claim 1,
wherein
said second protected amino acid has the structure of Formula III_{b} 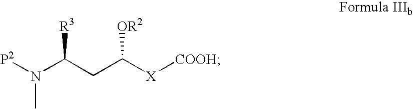
said first compound has the structure of Formula IV_{b} 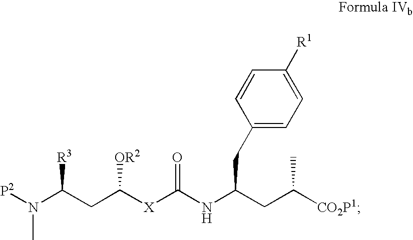
said second compound has the structure of Formula V_{b} 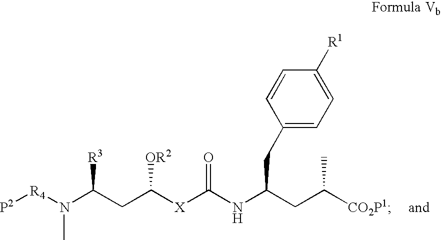
said reagent with structure of Formula VI is 1-methylpiperidine-2-carboxylic acid or acid protected derivative thereof; and
said compound with structure of Formula I has the structure of Formula I_{b}

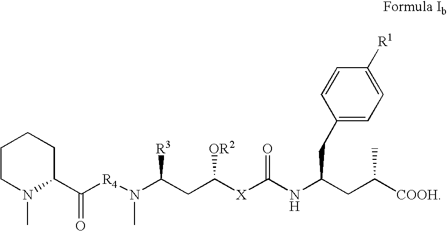

18. The process of claim 1 wherein said third protected amino acid is fluorenylmethoxycarbonyl-isoleucyl-fluoride.

19. The process of claim 1 wherein P¹ is allyl.

20. The process of claim 1 wherein P² and P³ of step (a) are independently t-butyloxycarbonyl.

21. The process of claim 1 wherein P² of step (b) is fluorenylmethoxycarbonyl.

22. The process of claim 1 wherein said acid protected derivative of reagent with structure R⁵—COOH is Mep-pentafluorophenyl ester.

23. The process of claim 1 wherein X is an optionally substituted heteroarylene.

24. The process of claim 1 wherein X is a five-membered heteroarylene.

25. The process of claim 24 wherein X comprises N and S.

26. The process of claim 1 wherein said second protected amino acid has the structure of Formula VIIₐ:

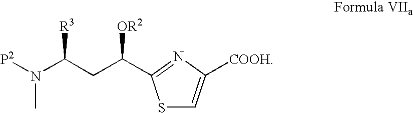

27. The process of claim 26 wherein said second protect amino acid has the structure of Formula VIIIa:

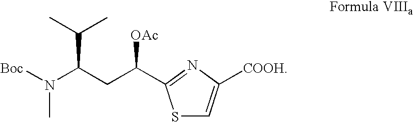

28. The process of claim 1 wherein said second protected amino acid has the structure of Formula VIIb:

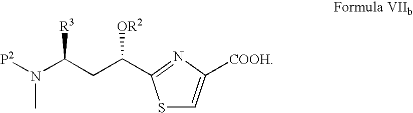

29. The process of claim 28 wherein said second protect amino acid has the structure of Formula VIII_{b}

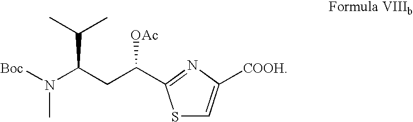

30. A process of the preparation of N-t-butyloxycarbonyl-N-methyl tubuvaline, said process comprising:
(a) reacting carbobenzoxyvaline under conditions suitable to form the methyl ester with structure of Formula IXa: 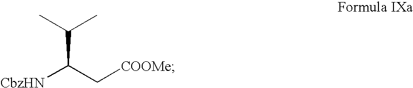
(b) reacting said methyl ester of step (a) under conditions suitable to form a t-butyldimethylsilyl ether with structure of Formula IXb: 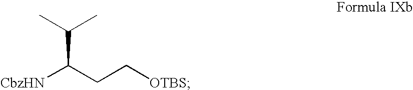
(c) reacting said t-butyldimethylsilyl ether under conditions suitable to form an alcohol with structure of Formula IXc: 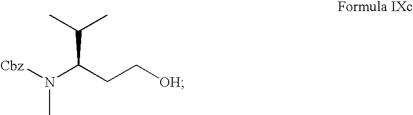
(d) reacting said alcohol under conditions suitable to form an aldehyde with structure of Formula IXd: 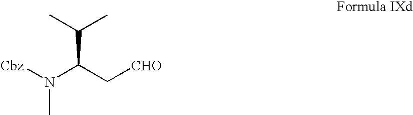
(e) reacting said aldehyde with a thiazole with structure of Formula IXe: 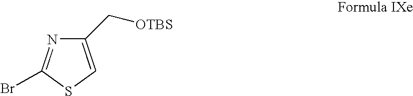
under Grignard conditions suitable to form epimeric compounds with structures of Formulae IXfₐ and IXf_{b}: 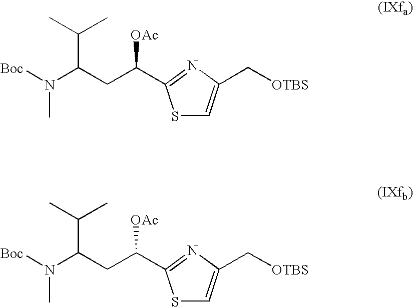
(f) reacting a compound resulting from step (e) under conditions suitable to form a deprotected alcohol with structure of either of Formulae IXgₐ and IXg_{b}: 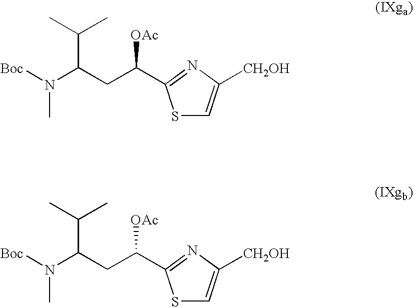
(g) reacting an alcohol resulting from step (f) under conditions suitable to form a N-t-butyloxycarbonyl-N-methyl tubuvaline with structure of either of Formulae IXhₐ and IXh_{b}:

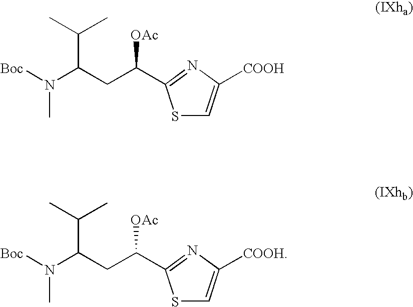

31. A process for the preparation of N¹⁴-desacetoxytubulysin H having the structure of Formula X: 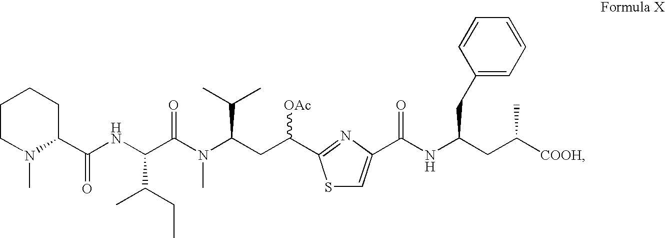
said process comprising:
(a) condensing a first protected amino acid with structure of Formula XI: 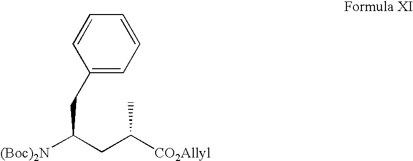
with a second protected amino acid with structure of Formula XII: 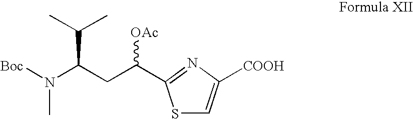
under conditions suitable to deprotect said first protected amino acid and to form a first compound with the structure of Formula XIII: 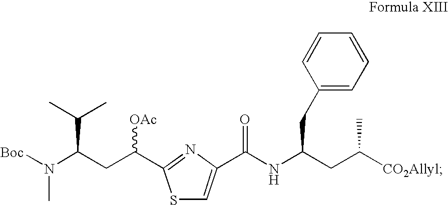
(b) reacting said first compound with a protected isoleucyl reactant under conditions suitable to deprotect said first compound and to form a second compound with the structure of Formula XIV: 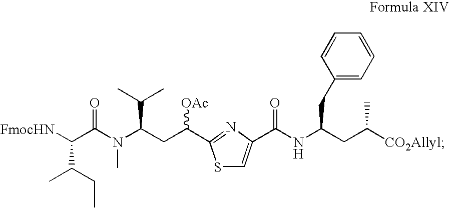
(c) reacting said second compound with acid protected 1-methylpiperidine-2-carboxylic acid under conditions suitable to deprotect said second compound and to form said N¹⁴-desacetoxytubulysin H with structure of Formula X.

32. The process according to claim 31,
wherein
said second protected amino acid has the structure of Formula IXhₐ 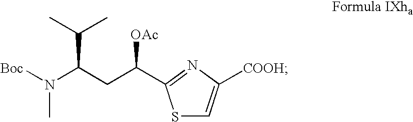
said first compound has the structure of Formula XIIIₐ 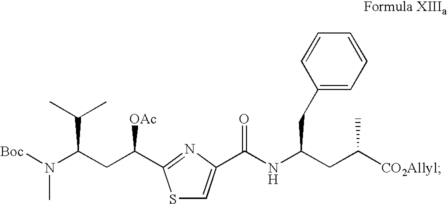
said second compound has the structure of Formula XIVₐ 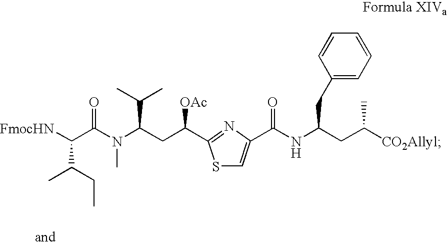
said N¹⁴-desacetoxytubulysin H has the structure of Formula Xₐ

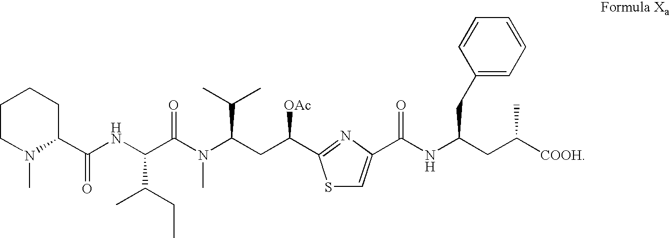

33. The process according to claim 31,
wherein
said second protected amino acid has the structure of Formula IXh_{b} 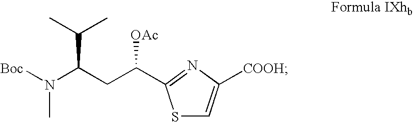
said first compound has the structure of Formula XIII_{b} 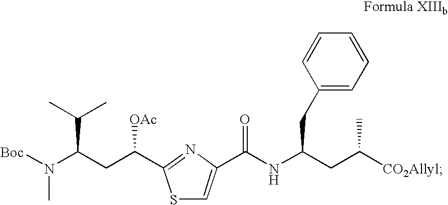
said second compound has the structure of Formula XIV_{b} 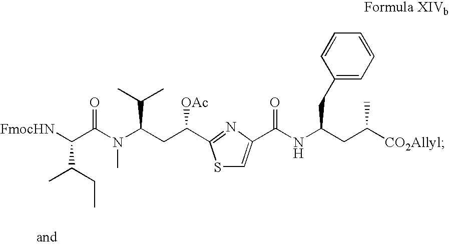
said N¹⁴-desacetoxytubulysin H has the structure of Formula X_{b}

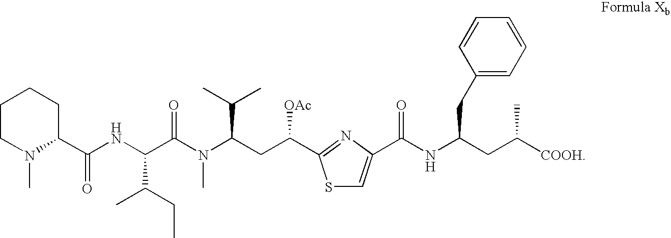

34. The compounds of N¹⁴-desacetoxytubulysin H, or a pharmaceutically acceptable salt thereof, having the structure of Formulae Xₐ or X_{b}:

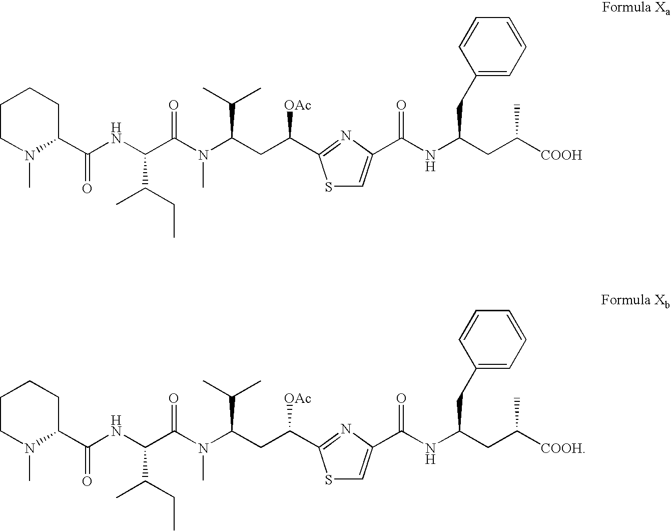

35. A process for the preparation of a compound having the structure of Formula XV: 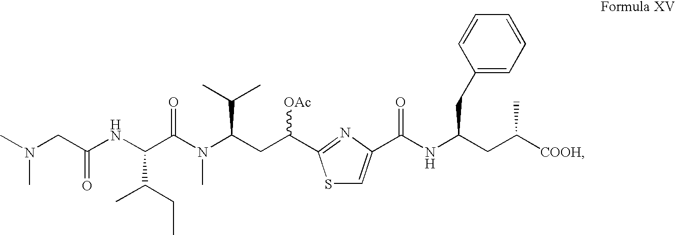
said process comprising:
(a) condensing a first protected amino acid with structure of Formula XI: 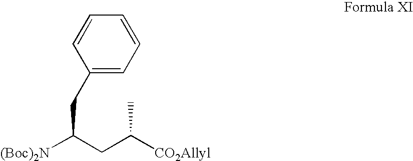
with a second protected amino acid with structure of Formula XII: 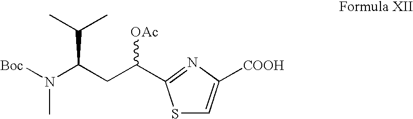
under conditions suitable to deprotect said first protected amino acid and to form a first compound with the structure of Formula XIII: 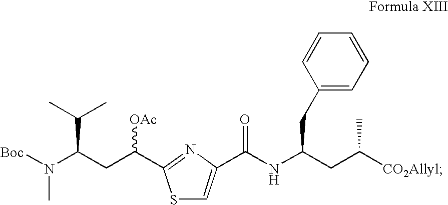
(b) reacting said first compound with a protected isoleucyl reactant under conditions suitable to deprotect said first compound and to form a second compound with the structure of Formula XIV: 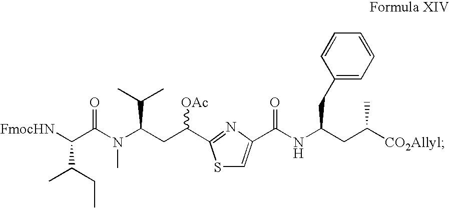
(c) reacting said second compound with 2-(dimethylamino)acetic acid or acid protected derivative thereof under conditions suitable to deprotect said second compound and to form said compound with structure of Formula XV.

36. The process according to claim 35,
wherein
said second protected amino acid has the structure of Formula IXhₐ 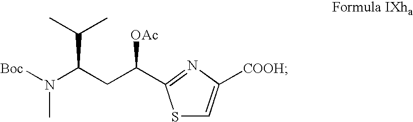
said first compound has the structure of Formula XIIIₐ 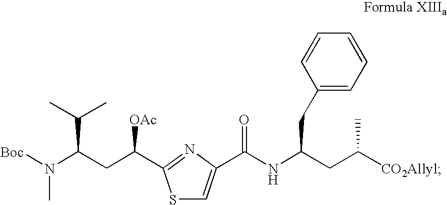
said second compound has the structure of Formula XIVₐ 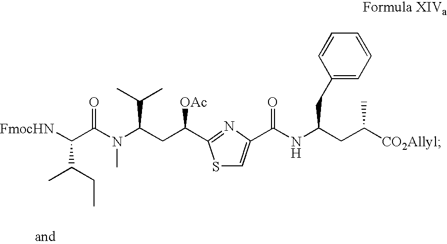
said compound having the structure of Formula XIV has the structure of Formula XVₐ

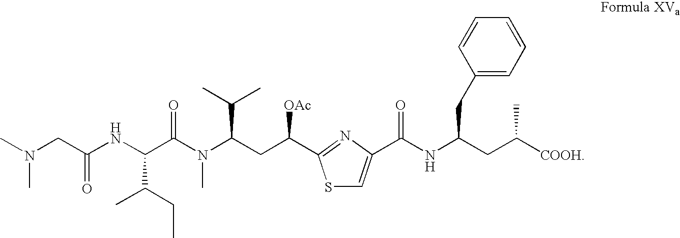

37. The process according to claim 35,
wherein
said second protected amino acid has the structure of Formula IXh_{b} 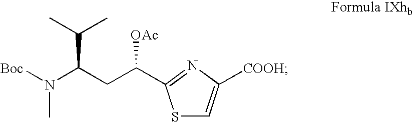
said first compound has the structure of Formula XIII_{b} 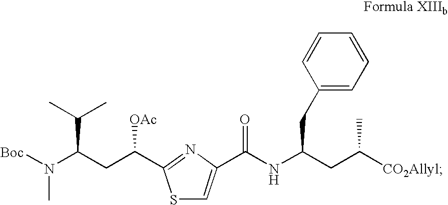
said second compound has the structure of Formula XIV_{b} 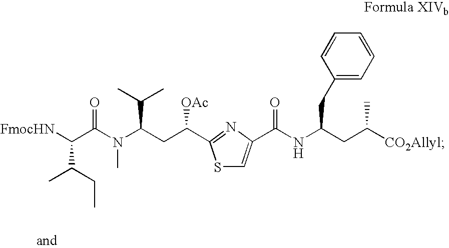
said compound having the structure of Formula XIV has the structure of Formula XV_{b}

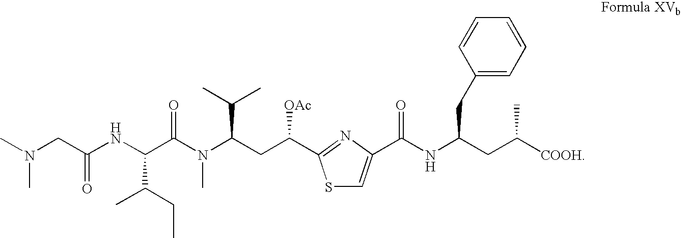

38. The compounds, or pharmaceutically acceptable salts thereof, having the structure of Formulae XVₐ or XV_{b}:

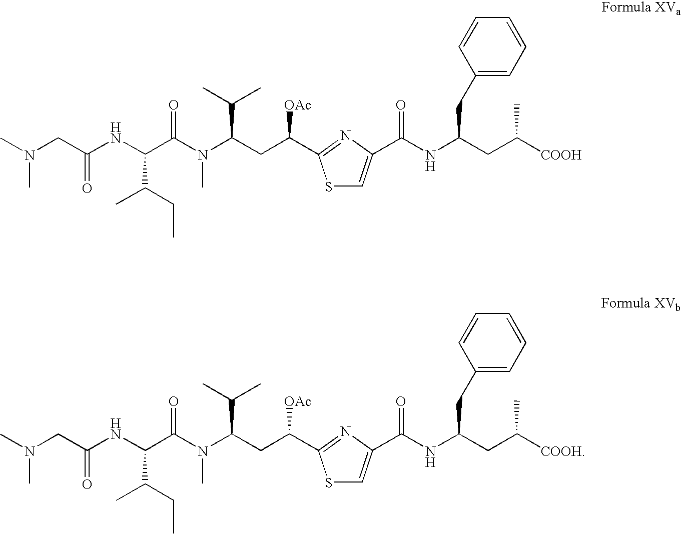

39. A method of inhibiting proliferation of a cell, said method comprising contacting a cell with a compound having the structure of Formula I 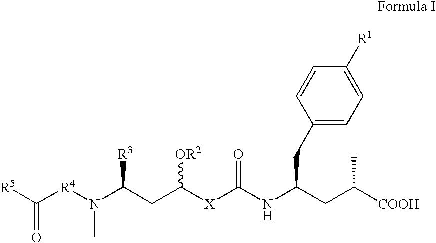
or pharmaceutically acceptable salt thereof,
wherein:
R¹ is H or OH;
R² is H or C(O)R⁶;
R³ is C₁₋₆ alkyl;
R⁴ is an amino acid selected from the group consisting of glycine, cysteine, alanine, histidine, asparagine, glutamine, arginine, threonine, valine, leucine, isoleucine, phenylalanine, tryptophan, serine, lysine, aspartic acid, methionine, glutamic acid, tyrosine, and optionally substituted derivatives thereof;
R⁵ is selected from the group consisting of optionally substituted heteroalkyl, optionally substituted heterocycloalkyl, and optionally substituted heteroaryl;
R⁶ is selected from the group consisting of H, optionally substituted alkyl, optionally substituted aryl, and optionally substituted heteroaryl; and
X is optionally substituted arylene or optionally substituted heteroarylene.

40. A method of screening for an inhibitor of cell proliferation, said method comprising:
(a) determining, in the presence and in the absence of a test compound and a cell, respectively, a level of proliferation of said cell,
wherein said test compound is a compound having the structure of Formula I 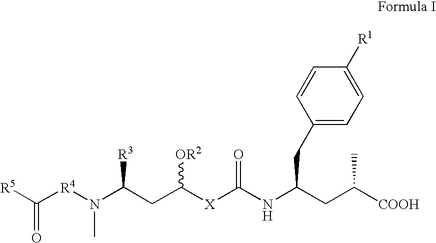
wherein:
R¹ is H or OH;
R² is H or C(O)R⁶;
R³ is C₁₋₆ alkyl;
R⁴ is an amino acid selected from the group consisting of glycine, cysteine, alanine, histidine, asparagine, glutamine, arginine, threonine, valine, leucine, isoleucine, phenylalanine, tryptophan, serine, lysine, aspartic acid, methionine, glutamic acid, tyrosine, and optionally substituted derivatives thereof;
R⁵ is selected from the group consisting of optionally substituted heteroalkyl, optionally substituted heterocycloalkyl, and optionally substituted heteroaryl;
R⁶ is selected from the group consisting of H, optionally substituted alkyl, optionally substituted aryl, and optionally substituted heteroaryl; and
X is optionally substituted arylene or optionally substituted heteroarylene;
(b) comparing the level of cell proliferation in the presence and in the absence of said test compound; and then
(c) ascertaining whether said test compound inhibits cell proliferation.

41. A process for the preparation of a compound with structure of Formula XVI: 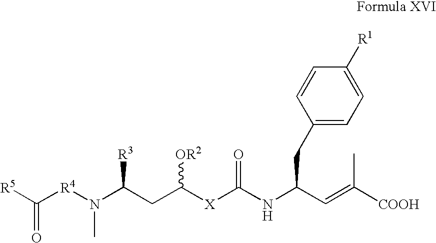
wherein
R¹ is H or OH;
R² is H or C(O)R⁶;
R³ is C₁₋₆ alkyl;
R⁴ is an amino acid selected from the group consisting of glycine, cysteine, alanine, histidine, asparagine, glutamine, arginine, threonine, valine, leucine, isoleucine, phenylalanine, tryptophan, serine, lysine, aspartic acid, methionine, glutamic acid, tyrosine, and optionally substituted derivatives thereof;
R⁵ is selected from the group consisting of optionally substituted heteroalkyl, optionally substituted heterocycloalkyl, and optionally substituted heteroaryl;
R⁶ is selected from the group consisting of H, optionally substituted alkyl, optionally substituted aryl, and optionally substituted heteroaryl; and
X is optionally substituted arylene or optionally substituted heteroarylene; said process comprising:
(a) condensing a first protected amino acid with structure of Formula XVII: 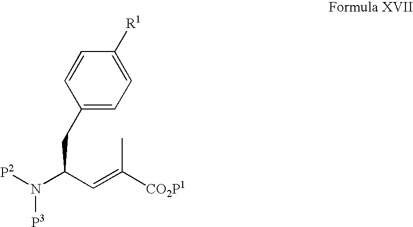
with a second protected amino acid with structure of Formula III: 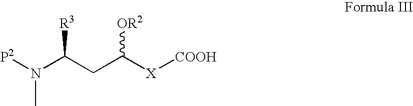
under conditions suitable to deprotect said first protected amino acid and to form a first compound with structure of Formula XVIII: 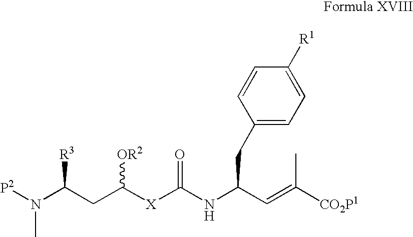
wherein
P¹ is an acid protecting group;
P² at each occurrence is independently an amine protecting group; and
P³ is H or P²;
(b) reacting said first compound with a third protected amino acid with structure P²—R⁴ under conditions suitable to deprotect said first compound and to form a second compound with structure of Formula XIX: 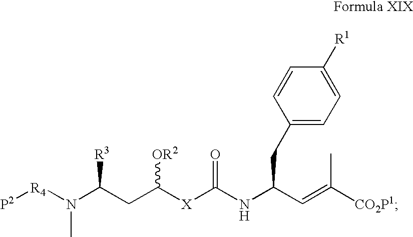
and
(c) reacting said second compound with a reagent with structure of Formula VI (i.e., R⁵—COOH) or acid protected derivative thereof under conditions suitable to deprotect said second compound and form said compound having structure of Formula XVI.

42. A compound with structure of Formula XVI: 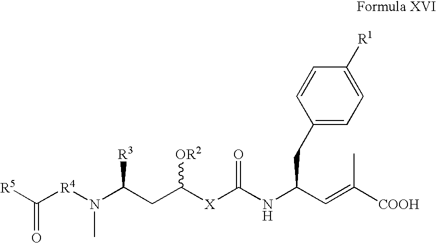
or pharmaceutically acceptable salt thereof,
wherein
R¹ is H or OH;
R² is H or C(O)R⁶;
R³ is C₁₋₆ alkyl;
R⁴ is an amino acid selected from the group consisting of glycine, cysteine, alanine, histidine, asparagine, glutamine, arginine, threonine, valine, leucine, isoleucine, phenylalanine, tryptophan, serine, lysine, aspartic acid, methionine, glutamic acid, tyrosine, and optionally substituted derivatives thereof;
R⁵ is selected from the group consisting of optionally substituted heteroalkyl, optionally substituted heterocycloalkyl, and optionally substituted heteroaryl;
R⁶ is selected from the group consisting of H, optionally substituted alkyl, optionally substituted aryl, and optionally substituted heteroaryl; and
X is optionally substituted arylene or optionally substituted heteroarylene.

43. The compounds of N¹⁴-desacetoxytubulysin H, or pharmaceutically acceptable salts thereof, having the structure of Formulae XXₐ or XX_{b}:

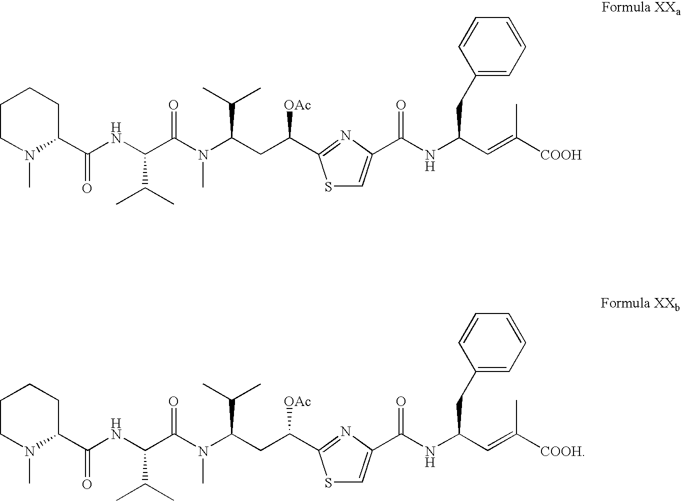

44. A process for the preparation of a compound with structure of Formula XXI: 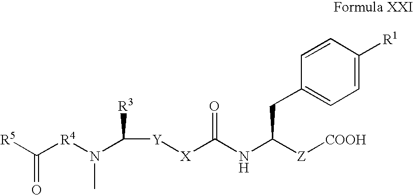
wherein
R¹ is H or O;
Y is alkylene, alkenylene or alkynylene;
Z is optionally substituted alkylene or optionally substituted alkenylene;
R³ is C₁₋₆ alkyl;
R⁴ is an amino acid selected from the group consisting of glycine, cysteine, alanine, histidine, asparagine, glutamine, arginine, threonine, valine, leucine, isoleucine, phenylalanine, tryptophan, serine, lysine, aspartic acid, methionine, glutamic acid, tyrosine, and optionally substituted derivatives thereof;
R⁵ is selected from the group consisting of optionally substituted heteroalkyl, optionally substituted heterocycloalkyl, and optionally substituted heteroaryl; and
X is optionally substituted arylene or optionally substituted heteroarylene;
said process comprising:
(a) condensing a first protected amino acid with structure of Formula XXVIII: 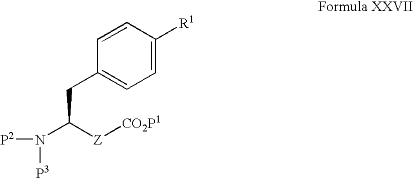
with a second protected amino acid with structure of Formula XXII: 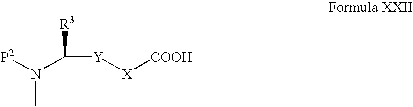
under conditions suitable to deprotect said first protected amino acid and to form a first compound with structure of Formula XXVIII: 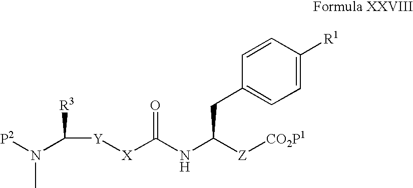
wherein
P¹ is an acid protecting group,
P² at each occurrence is independently an amine protecting group, and
P³ is H or P²;
(b) reacting said first compound with a third protected amino acid with structure P²—R⁴ under conditions suitable to deprotect said first compound and to form a second compound with structure of Formula XXIX: 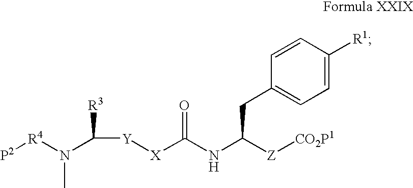
and
(c) reacting said second compounds with a reagent with structure of Formula VI (i.e., R⁵—COOH) or acid protected derivative thereof under conditions suitable to deprotect said second compound and form said compound having structure of Formula XXI.

45. A compound with structure of Formula XXI: 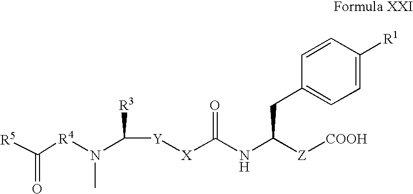
or pharmaceutically acceptable salt thereof,
wherein
R¹ is H or OH;
Y is alkylene, alkenylene or alkynylene;
Z is optionally substituted alkylene or optionally substituted alkenylene
R³ is C₁₋₆ alkyl;
R⁴ is an amino acid selected from the group consisting of glycine, cysteine, alanine, histidine, asparagine, glutamine, arginine, threonine, valine, leucine, isoleucine, phenylalanine, tryptophan, serine, lysine, aspartic acid, methionine, glutamic acid, tyrosine, and optionally substituted derivatives thereof,
R⁵ is selected from the group consisting of optionally substituted heteroalkyl, optionally substituted heterocycloalkyl, and optionally substituted heteroaryl; and
X is optionally substituted arylene or optionally substituted heteroarylene.

46. The compounds of N¹⁴-desacetoxytubulysin H, or pharmaceutically acceptable salts thereof, having the structure of Formulae XXV, XXVI, XXX, XXXI, XXXII XXXIII or XXXIV:

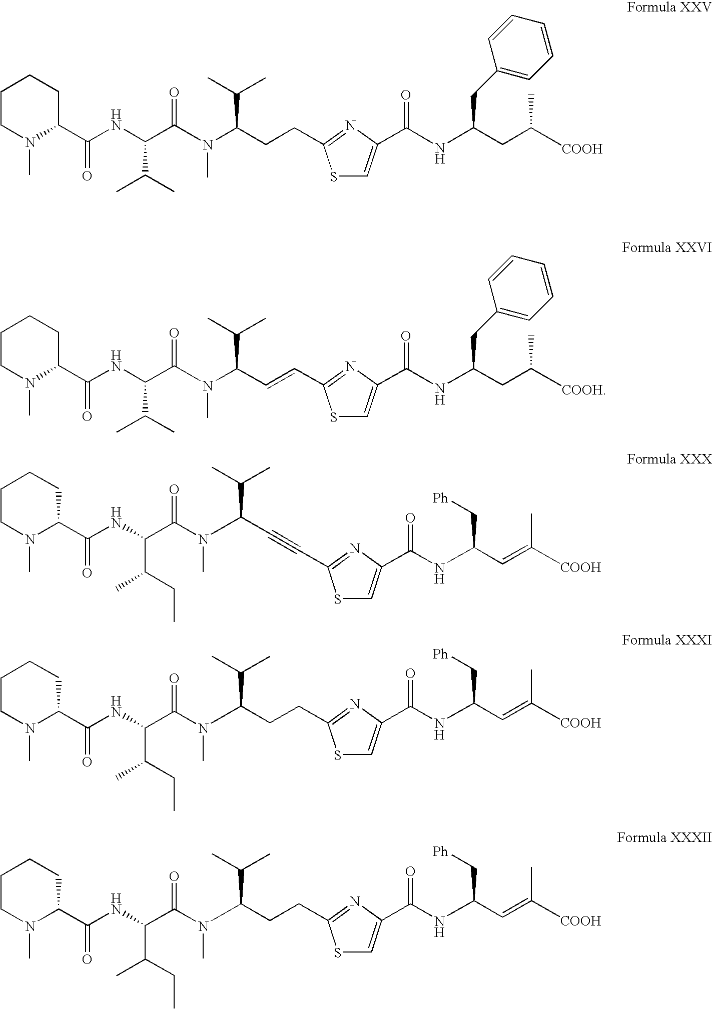

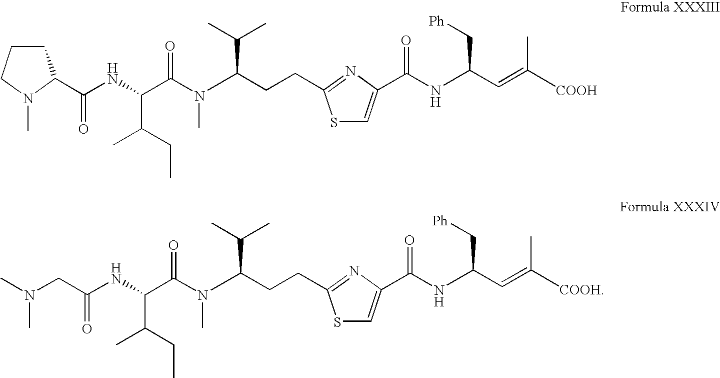

47. A method of inhibiting proliferation of a cell, said method comprising contacting a cell with a compound having the structure of Formula XVI 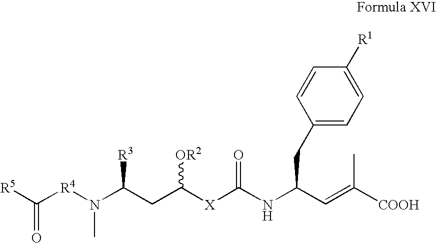
wherein
R¹ is H or OH;
R² is H or C(O)R⁶;
R³ is C₁₋₆ alkyl;
R⁴ is an amino acid selected from the group consisting of glycine, cysteine, alanine, histidine, asparagine, glutamine, arginine, threonine, valine, leucine, isoleucine, phenylalanine, tryptophan, serine, lysine, aspartic acid, methionine, glutamic acid, tyrosine, and optionally substituted derivatives thereof;
R⁵ is selected from the group consisting of optionally substituted heteroalkyl, optionally substituted heterocycloalkyl, and optionally substituted heteroaryl;
R⁶ is selected from the group consisting of H, optionally substituted alkyl, optionally substituted aryl, and optionally substituted heteroaryl; and
X is optionally substituted arylene or optionally substituted heteroarylene.

48. A method of inhibiting proliferation of a cell, said method comprising contacting a cell with a compound having the structure of Formula XXI 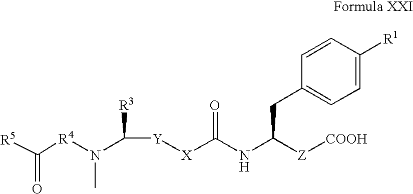
wherein
R¹ is H or OH;
Y is alkylene, alkenylene or alkynylene;
Z is optionally substituted alkylene or optionally substituted alkenylene;
R³ is C₁₋₆ alkyl;
R⁴ is an amino acid selected from the group consisting of glycine, cysteine, alanine, histidine, asparagine, glutamine, arginine, threonine, valine, leucine, isoleucine, phenylalanine, tryptophan, serine, lysine, aspartic acid, methionine, glutamic acid, tyrosine, and optionally substituted derivatives thereof;
R⁵ is selected from the group consisting of optionally substituted heteroalkyl, optionally substituted heterocycloalkyl, and optionally substituted heteroaryl; and
X is optionally substituted arylene or optionally substituted heteroarylene.

49. A method of screening for an inhibitor of cell proliferation, said method comprising:
(a) determining, in the presence and in the absence of a test compound and a cell, respectively, a level of proliferation of said cell,
wherein said test compound is a compound having the structure of Formula XVI: 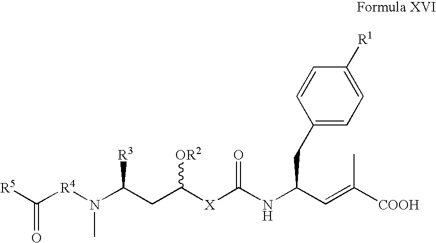
wherein
R¹ is H or OH;
R² is H or C(O)R⁶;
R³ is C₁₋₆ alkyl;
R⁴ is an amino acid selected from the group consisting of glycine, cysteine, alanine, histidine, asparagine, glutamine, arginine, threonine, valine, leucine, isoleucine, phenylalanine, tryptophan, serine, lysine, aspartic acid, methionine, glutamic acid, tyrosine, and optionally substituted derivatives thereof;
R⁵ is selected from the group consisting of optionally substituted heteroalkyl, optionally substituted heterocycloalkyl, and optionally substituted heteroaryl;
R⁶ is selected from the group consisting of H, optionally substituted alkyl, optionally substituted aryl, and optionally substituted heteroaryl; and
X is optionally substituted arylene or optionally substituted heteroarylene;
(b) comparing the level of cell proliferation in the presence and in the absence of said test compound; and then
(c) ascertaining whether said test compound inhibits cell proliferation.

50. A method of screening for an inhibitor of cell proliferation, said method comprising:
(a) determining, in the presence and in the absence of a test compound and a cell, respectively, a level of proliferation of said cell,
wherein said test compound is a compound having the structure of Formula XXI: 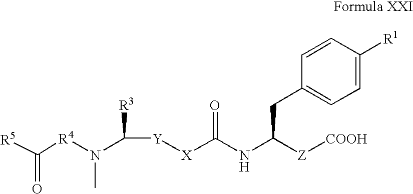
wherein
R¹ is H or OH;
Y is alkylene, alkenylene or alkynylene;
Z is optionally substituted alkylene or optionally substituted alkenylene;
R³ is C₁₋₆ alkyl;
R⁴ is an amino acid selected from the group consisting of glycine, cysteine, alanine, histidine, asparagine, glutamine, arginine, threonine, valine, leucine, isoleucine, phenylalanine, tryptophan, serine, lysine, aspartic acid, methionine, glutamic acid, tyrosine, and optionally substituted derivatives thereof;
R⁵ is selected from the group consisting of optionally substituted heteroalkyl, optionally substituted heterocycloalkyl, and optionally substituted heteroaryl; and
X is optionally substituted arylene or optionally substituted heteroarylene;
(b) comparing the level of cell proliferation in the presence and in the absence of said test compound; and then
(c) ascertaining whether said test compound inhibits cell proliferation.

51. The process according to claim 44,
wherein
said first protected amino acid has the structure of Formula II 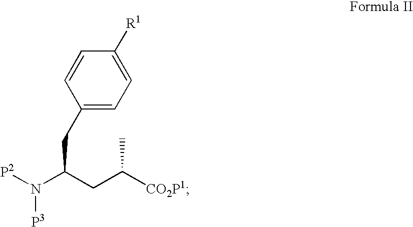
said first compound has the structure of Formula XXIII 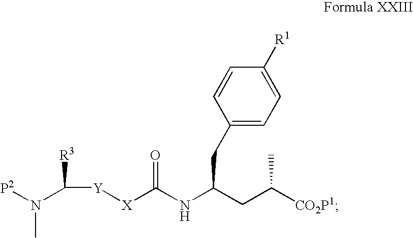
said second compound has the structure of Formula XXIV 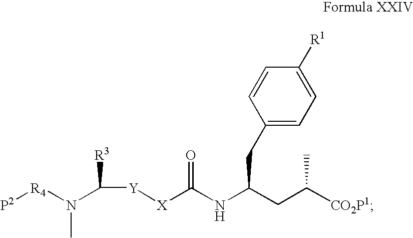
and said third compound has the structure of Formula XXXV.

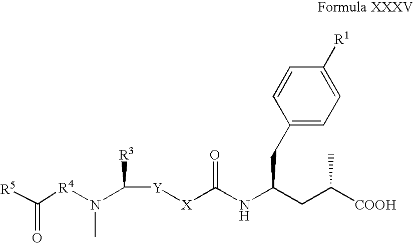

52. A compound with structure of Formula XXXV: 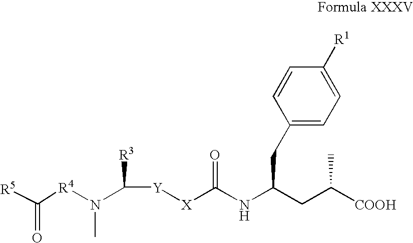
or pharmaceutically acceptable salt thereof,
wherein
R¹ is H or OH;
Y is alkylene or alkenylene;
R³ is C₁₋₆ alkyl;
R⁴ is an amino acid selected from the group consisting of glycine, cysteine, alanine, histidine, asparagine, glutamine, arginine, threonine, valine, leucine, isoleucine, phenylalanine, tryptophan, serine, lysine, aspartic acid, methionine, glutamic acid, tyrosine, and optionally substituted derivatives thereof,
R⁵ is selected from the group consisting of optionally substituted heteroalkyl, optionally substituted heterocycloalkyl, and optionally substituted heteroaryl; and
X is optionally substituted arylene or optionally substituted heteroarylene.

53. A method of inhibiting proliferation of a cell, said method comprising contacting a cell with a compound having the structure of Formula XXXV 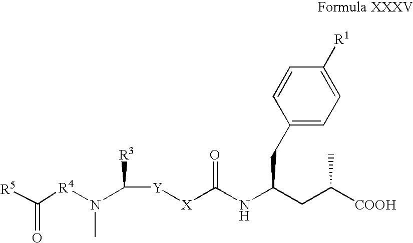
wherein
R¹ is H or OH;
Y is alkylene or alkenylene;
R³ is C₁₋₆ alkyl;
R⁴ is an amino acid selected from the group consisting of glycine, cysteine, alanine, histidine, asparagine, glutamine, arginine, threonine, valine, leucine, isoleucine, phenylalanine, tryptophan, serine, lysine, aspartic acid, methionine, glutamic acid, tyrosine, and optionally substituted derivatives thereof;
R⁵ is selected from the group consisting of optionally substituted heteroalkyl, optionally substituted heterocycloalkyl, and optionally substituted heteroaryl; and
X is optionally substituted arylene or optionally substituted heteroarylene.

54. A method of screening for an inhibitor of cell proliferation, said method comprising:
(a) determining, in the presence and in the absence of a test compound and a cell, respectively, a level of proliferation of said cell,
wherein said test compound is a compound having the structure of Formula XXXV: 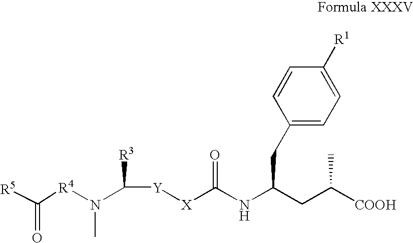
wherein
R¹ is H or OH;
Y is alkylene or alkenylene;
R³ is C₁₋₆ alkyl;
R⁴ is an amino acid selected from the group consisting of glycine, cysteine, alanine, histidine, asparagine, glutamine, arginine, threonine, valine, leucine, isoleucine, phenylalanine, tryptophan, serine, lysine, aspartic acid, methionine, glutamic acid, tyrosine, and optionally substituted derivatives thereof;
R⁵ is selected from the group consisting of optionally substituted heteroalkyl, optionally substituted heterocycloalkyl, and optionally substituted heteroaryl; and
X is optionally substituted arylene or optionally substituted heteroarylene;
(b) comparing the level of cell proliferation in the presence and in the absence of said test compound; and then
(c) ascertaining whether said test compound inhibits cell proliferation.
